(19) United States
(12) Patent Application Publication
(10) Pub. No.: US 2025/0346599 A1
(43) Pub. Date: Nov. 13, 2025

(54) TRICYCLIC PHTHALAZINONE PARP INHIBITORS AND METHODS OF USE

(71) Applicant: Valo Health, Inc., Lexington, MA (US)

(72) Inventors: Eric Lars Stangeland, Lexington, MA (US); Ann-Marie Campbell, Lexington, MA (US); John Griffin, Lexington, MA (US)

(21) Appl. No.: 18/872,713

(22) PCT Filed: Jun. 7, 2023
(86) PCT No.: PCT/US2023/024708

§ 371 Date: Dec. 6, 2024

Related U.S. Application Data
(60) Provisional application No. 63/350,366, filed on Jun. 8, 2022.

(51) Int. Cl. C07D 471/06; A61K 31/502

(52) U.S. Cl. CPC C07D 471/06; A61K 31/502

(57) ABSTRACT

The present disclosure relates to compounds according to Formula I or a pharmaceutically acceptable salt and/or solvate thereof, as well as compositions including such compounds and uses thereof, where X¹ is H, F, or Cl; X² is NH or N—R³; one of R¹ and R² is H, halo, alkyl, alkenyl, alkynyl, cycloalkyl, or heterocyclyl, and the remaining one of R¹ and R² is aryl, heteroaryl, or non-aromatic heterocyclyl; and R³ is alkyl, cycloalkyl, alkylenyl, and non-aromatic heterocyclyl. Among other things, the present disclosure evidences compounds of the present disclosure penetrate the central nervous system allowing for treatment of central nervous system cancers.

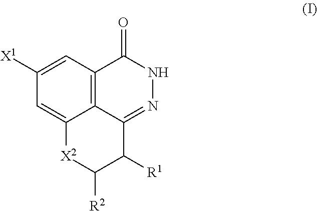

### CROSS-REFERENCE TO RELATED APPLICATIONS

This application claims the benefit of and priority to Provisional Patent Application No. 63/350,366, filed Jun. 8, 2022, which is incorporated by reference herein in its entirety for any and all purposes.

### FIELD

The present technology is directed to compounds, compositions, and methods related to treatment of cancer, especially central nervous system cancers, and uses thereof.

### SUMMARY

In an aspect, the present technology provides a compound according to Formula I

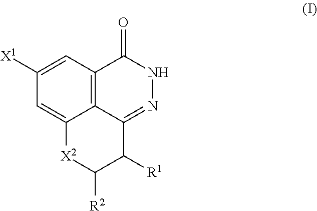

or a pharmaceutically acceptable salt and/or solvate thereof, where X¹ is H, F, or Cl; X² is NH or N—R³; one of R¹ and R² is H, halo, alkyl, alkenyl, alkynyl, cycloalkyl, or heterocyclyl, and the remaining one of R¹ and R² is aryl, heteroaryl, or non-aromatic heterocyclyl; and R³ is alkyl, cycloalkyl, alkylenyl, and non-aromatic heterocyclyl.

In an aspect, a composition is provided that includes a compound of any embodiment disclosed herein, a pharmaceutically acceptable carrier or one or more excipients, fillers or agents (collectively referred to hereafter as “pharmaceutically acceptable carrier” unless otherwise indicated and/or specified).

In a related aspect, a medicament for treating a cancer in a subject is provided that includes a compound of any embodiment disclosed herein and optionally a pharmaceutically acceptable carrier.

In a related aspect, a pharmaceutical composition is provided that includes (i) an effective amount of a compound of any embodiment disclosed herein, wherein the effective amount of the compound is effective to treat the cancer; and (ii) a pharmaceutically acceptable carrier.

In a related aspect, a pharmaceutical composition is provided that includes (i) an effective amount of a compound of any embodiment disclosed herein, where the compound is present in an amount effective to treat a cancer when combined with a second cancer therapy; and (ii) a pharmaceutically acceptable carrier.

In further related aspects, the present technology provides methods including a compound of any aspect or embodiment disclosed herein and/or a composition of any embodiment disclosed herein and/or a medicament of any embodiment disclosed herein. Such methods include a method of treating a subject suffering from a, where the method includes administering to the subject an effective amount of a compound of any embodiment disclosed herein and an effective amount of a second cancer therapy.

### DETAILED DESCRIPTION

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

The phrase “and/or” as used in the present disclosure will be understood to mean any one of the recited members individually or a combination of any two or more thereof—for example, “A, B, and/or C” would mean “A, B, C, A and B, A and C, B and C, or the combination of A, B, and C.”

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SF₅), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; and nitriles (i.e., CN).

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Cycloalkyl groups may be substituted or unsubstituted. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. Cycloalkylalkyl groups may be substituted or unsubstituted. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. Cycloalkenyl groups may be substituted or unsubstituted. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Cycloalkenylalkyl groups may be substituted or unsubstituted. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups may be substituted or unsubstituted. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, and —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. Heterocyclyl groups may be substituted or unsubstituted. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. The phrase includes heterocyclyl groups that have other groups, such as alkyl, oxo or halo groups, bonded to one of the ring members, referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl,azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3—, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups may be substituted or unsubstituted. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Heterocyclylalkyl groups may be substituted or unsubstituted. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Heteroaralkyl groups may be substituted or unsubstituted. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl groups and —O—C(O)-alkyl groups, each containing 2-5 carbon atoms. Similarly, “aryloyl” and “aryloyloxy” refer to —C(O)-aryl groups and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylate” as used herein refers to a —COOH group.

The term “ester” as used herein refers to —COOR⁷⁰ and —C(O)O-G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T.W.; Wuts, P. G. M., John Wiley & Sons, New York, NY, (3rd Edition, 1999) which can be added or removed using the procedures set forth therein and which is hereby incorporated by reference in its entirety and for any and all purposes as if fully set forth herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR⁷⁸R⁷⁹ and —NR⁷⁸SO₂R⁷⁹ groups, respectively. R⁷⁸ and R⁷⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while “sulfides” include —SR⁸⁰ groups, “sulfoxides” include —S(O)R⁸¹ groups, “sulfones” include —SO₂R⁸² groups, and “sulfonyls” include —SO₂OR⁸³. R⁸⁰, R⁸¹, R⁸², and R⁸³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR⁸⁴—C(O)—NR⁸⁵R⁸⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁹⁴)═C(R⁹⁵)NR⁹⁶R⁹⁷ and —NR⁹⁴C(R⁹⁵)═C(R⁹⁶)R⁹⁷, wherein R⁹⁴, R⁹⁵, R⁹⁶ and R⁹⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxyl” as used herein can refer to —OH or its ionized form, —O⁻. A “hydroxyalkyl” group is a hydroxyl-substituted alkyl group, such as HO—CH₂—.

The term “imide” refers to —C(O)NR⁹⁸C(O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR¹⁰⁰(NR¹⁰¹) and —N(CR^{1OO}R¹⁰¹) groups, wherein R¹⁰⁰ and R¹⁰¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

The term “nitro” as used herein refers to an —NO₂ group.

The term “trifluoromethyl” as used herein refers to —CF₃.

The term “trifluoromethoxy” as used herein refers to —OCF₃.

The term “azido” refers to —N₃.

The term “trialkyl ammonium” refers to a —N(alkyl)₃ group. A trialkylammonium group is positively charged and thus typically has an associated anion, such as halogen anion.

The term “isocyano” refers to —NC.

The term “isothiocyano” refers to —NCS.

The term “pentafluorosulfanyl” refers to —SF₅.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

As understood by one of ordinary skill in the art, “molecular weight” (also known as “relative molar mass”) is a dimensionless quantity but is converted to molar mass by multiplying by 1 gram/mole or by multiplying by 1 Da—for example, a compound with a weight-average molecular weight of 5,000 has a weight-average molar mass of 5,000 g/mol and a weight-average molar mass of 5,000 Da.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism, and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

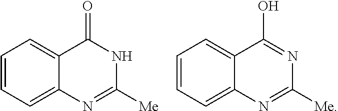

As another example, guanidines may exhibit the following isomeric forms in protic organic solution, also referred to as tautomers of each other:

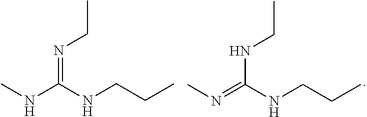

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

Throughout this disclosure, various publications, patents and published patent specifications are referenced by an identifying citation. Also within this disclosure are Arabic numerals referring to referenced citations, the full bibliographic details of which are provided subsequent to the Examples section. The disclosures of these publications, patents and published patent specifications are hereby incorporated by reference into the present disclosure to more fully describe the present technology.

### The Present Technology

Poly ADP ribose polymerase 1 (PARP1) is a member of a family of proteins involved in myriad cellular processes such as DNA repair, genomic stability, and programmed cell death. PARP1 is an ADP-ribosyltransferase that uses NAD+ as a substrate to modify proteins—including itself—by PARylation and detects single-strand DNA breaks, playing a significant role in homologous recombination and DNA repair by signaling the enzymatic processes involved in repairing single-strand DNA breaks.

Several PARP1 inhibitors have been approved for the treatment of breast and ovarian cancers deficient in other mechanisms of DNA repair, and further PARP inhibitors are currently in clinical trials for the treatment of ovarian cancer, pancreatic and biliary tract malignancies, glioblastoma, lung cancers, and prostatic cancers. BRCA1/2-deficient cancers are exquisitely sensitive to PARP1 inhibition, where PARP inhibition is lethal in cells with loss-of-function mutations in BRCA1 and BRCA2. Reduced homologous recombination (HR) capacity through other mechanisms also sensitizes cells to PARP inhibitors. Cells lacking defects in DNA repair are typically 1000-fold less susceptible to PARP inhibitors than those with such defects. PARP inhibitors reduce PARylation and trap PARP1 on DNA causing replication fork collapse and cell death.

Yet current PARP1 inhibitors such as talazoparib, olaparib, rucaparib, and niraparib do not have good central nervous system (CNS) penetration, and veliparib is not efficacious in most tumors. See, e.g., Gupta, Shiv K., et a.[“PARP inhibitors for sensitization of alkylation chemotherapy in glioblastoma: impact of blood-brain barrier and molecular heterogeneity” Frontiers in oncology 8 (2019): 670; Kizilbash, S. H. et al. “Restricted delivery of talazoparib across the blood-brain barrier limits the sensitizing effects of PARP inhibition on Temozolomide therapy in glioblastoma” Mol. Cancer Ther, 16 (2017): 2735-2746.

Accordingly, there is a need for PARP inhibitors with improved central nervous system penetration in order to treat brain cancers such as a glioblastoma, a neuroblastoma, and a medulloblastoma.

The present technology responds to these needs as well as provides additional advantages. According to internal proprietary predictive models, compounds of the present technology were predicted to exhibit desirable blood brain barrier (BBB) permeability. These proprietary predictive models were further verified by in vitro assays well-established to be predictive of BBB permeability in living subject as well as further verified by in vivo experiments. Accordingly, the present technology provides compounds with advantageously improved central nervous system penetration, as well as compositions and methods especially suited to treatment of central nervous system cancers.

Thus, in an aspect, the present technology provides a compound according to Formula I

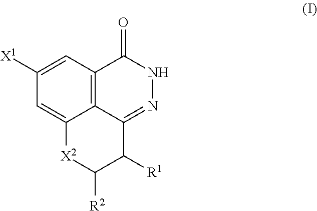

or a pharmaceutically acceptable salt and/or solvate thereof, where X¹ is H, F, or Cl; X² is NH or N—R³; one of R¹ and R² is H, halo, alkyl, alkenyl, alkynyl, cycloalkyl, or heterocyclyl, and the remaining one of R¹ and R² is aryl, heteroaryl, or non-aromatic heterocyclyl; and R³ is alkyl, cycloalkyl, alkylenyl, and non-aromatic heterocyclyl. For ease of reference, the compounds included in any aspect or embodiment herein may be referred to anywhere in this disclosure as “a compound of the present technology,” “compounds of the present technology,” or the like. Similarly for ease of reference, the compositions, medicaments, and pharmaceutical compositions of the present technology may collectively be referred to herein as “compositions,” “compositions of the present technology,” or the like.

In any embodiment herein, the compound of Formula I may be of Formula IA

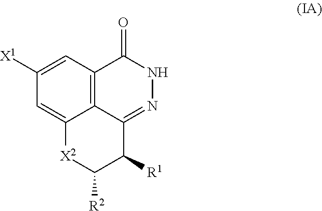

or a pharmaceutically acceptable salt and/or solvate thereof.

In any embodiment herein, the compound of Formula I may be of Formula IB

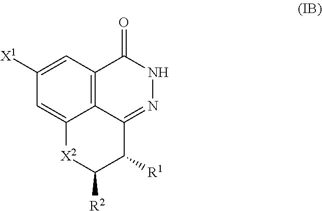

or a pharmaceutically acceptable salt and/or solvate thereof.

In any embodiment herein, it may be that R¹ is aryl, heteroaryl, or non-aromatic heterocyclyl; and R² is H, halo, alkyl, alkenyl, alkynyl, cycloalkyl, or non-aromatic heterocyclyl. In any embodiment herein, it may be that R¹ is aryl, heteroaryl, or non-aromatic heterocyclyl; and R² is H, alkyl, cycloalkyl, or non-aromatic heterocyclyl. In any embodiment herein, it may be that X¹ is F. In any embodiment herein, it may be that X² is NH.

In an aspect, a composition is provided that includes a compound of any embodiment disclosed herein, a pharmaceutically acceptable carrier or one or more excipients, fillers or agents (collectively referred to hereafter as “pharmaceutically acceptable carrier” unless otherwise indicated and/or specified). In a related aspect, a medicament for treating a cancer in a subject is provided that includes a compound of any embodiment disclosed herein and optionally a pharmaceutically acceptable carrier. The medicament of any embodiment herein may include an effective amount of the compound for treating the cancer when combined with a second cancer therapy, such as radiation therapy, a monoclonal antibody, and/or a chemotherapeutic. In any embodiment herein, the cancer may be a breast cancer, an ovarian cancer, a pancreatic cancer, a biliary tract cancer, a lung cancer, a prostatic cancer, and/or a CNS cancer (such as a brain cancer). The CNS cancer may be a glioblastoma, a neuroblastoma, and/or a medulloblastoma. In a related aspect, a pharmaceutical composition is provided that includes (i) an effective amount of a compound of any embodiment disclosed herein, wherein the effective amount of the compound is effective to treat a cancer; and (ii) a pharmaceutically acceptable carrier. In any embodiment herein, the cancer may be a breast cancer, an ovarian cancer, a pancreatic cancer, a biliary tract cancer, a lung cancer, a prostatic cancer, and/or a CNS cancer (such as a brain cancer). In any embodiment herein, the cancer may be a breast cancer, an ovarian cancer, a pancreatic cancer, a biliary tract cancer, a lung cancer, a prostatic cancer, and/or a CNS cancer (such as a brain cancer). In a related aspect, a pharmaceutical composition is provided that includes (i) an effective amount of a compound of any embodiment disclosed herein, where the compound is present in an amount effective to treat a cancer when combined with second cancer therapy (such as radiation therapy, a monoclonal antibody, and/or a chemotherapeutic); and (ii) a pharmaceutically acceptable carrier. In any embodiment herein, the cancer may be a breast cancer, an ovarian cancer, a pancreatic cancer, a biliary tract cancer, a lung cancer, a prostatic cancer, and/or a CNS cancer (such as a brain cancer). In further related aspects, the present technology provides methods including a compound of any aspect or embodiment disclosed herein and/or a composition of any embodiment disclosed herein and/or a medicament of any embodiment disclosed herein.

“Effective amount” refers to the amount of a compound or composition required to produce a desired effect. One example of an effective amount includes amounts or dosages that yield acceptable toxicity and bioavailability levels for therapeutic (pharmaceutical) use including, but not limited to, reduction of a tumor mass. In any aspect or embodiment disclosed herein (collectively referred to herein as “any embodiment herein,” “any embodiment disclosed herein,” or the like) of the compositions, pharmaceutical compositions, and methods including compounds of the present technology, the effective amount may be an amount effective in treating a cancer (such as a breast cancer, an ovarian cancer, a pancreatic cancer, a biliary tract cancer, a lung cancer, a prostatic cancer, and/or a CNS cancer), treating a tumor, and/or shrinking a tumor. By way of example, the effective amount of any embodiment herein including a compound of the present technology may be from about 0.01 g to about 200 mg of the compound (such as from about 0.1 g to about 50 mg of the compound or about 10 g to about 20 mg of the compound). The methods and uses according to the present technology may include an effective amount of a compound of any embodiment disclosed herein. In any aspect or embodiment disclosed herein, the effective amount may be determined in relation to a subject. As used herein, a “subject” or “patient” is a mammal, such as a cat, dog, rodent or primate. Typically the subject is a human, and, preferably, a human suffering from or suspected of suffering from pain. The term “subject” and “patient” can be used interchangeably.

Thus, the instant present technology provides pharmaceutical compositions and medicaments including a compound of any embodiment disclosed herein (or a composition of any embodiment disclosed herein) and a pharmaceutically acceptable carrier. The compositions may be used in the methods and treatments described herein. The pharmaceutical composition may be packaged in unit dosage form. The unit dosage form may be effective in treating a cancer (such as a breast cancer, an ovarian cancer, a pancreatic cancer, a biliary tract cancer, a lung cancer, a prostatic cancer, and/or a CNS cancer). The unit dosage form may be effective in treating a tumor by reducing a tumor volume when administered to a subject in need thereof. Generally, a unit dosage including a compound of the present technology will vary depending on patient considerations. Such considerations include, for example, age, protocol, condition, sex, extent of disease, contraindications, concomitant therapies and the like. An exemplary unit dosage based on these considerations may also be adjusted or modified by a physician skilled in the art. For example, a unit dosage for a patient comprising a compound of the present technology may vary from 1×10⁻⁴ g/kg to 1 g/kg, preferably, 1×10⁻³ g/kg to 1.0 g/kg. Dosage of a compound of the present technology may also vary from 0.01 mg/kg to 100 mg/kg or, preferably, from 0.1 mg/kg to 10 mg/kg. Suitable unit dosage forms, include, but are not limited to parenteral solutions, oral solutions, powders, tablets, pills, gelcaps, capsules, lozenges, suppositories, patches, nasal sprays, injectables, implantable sustained-release formulations, mucoadherent films, topical varnishes, lipid complexes, liquids, etc.

The pharmaceutical compositions and medicaments may be prepared by mixing one or more compounds and/or compositions of the present technology with pharmaceutically acceptable carriers, excipients, binders, diluents or the like. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more compounds of the instant present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Compounds of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars and/or sugar alcohols. Aqueous and nonaqueous (e.g., in a fluorocarbon propellant) aerosols are typically used for delivery of compounds of the present technology by inhalation.

Dosage forms for the topical (including buccal and sublingual) or transdermal administration of compounds of the present technology include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the compounds of the present technology across the skin. The rate of such flux can be controlled by either providing a rate controlling membrane (e.g., as part of a transdermal patch) or dispersing the compound in a polymer matrix or gel.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Those skilled in the art are readily able to determine an effective amount by simply administering a compound of the present technology to a patient in increasing amounts until, for example, there is a reduction in the mass of a tumor in a subject. The compounds of the present technology can be administered to a patient at dosage levels in the range of about 0.1 to about 1,000 mg per day. For a normal human adult having a body weight of about 70 kg, a dosage in the range of about 0.01 to about 100 mg per kg of body weight per day is sufficient. The specific dosage used, however, can vary or may be adjusted as considered appropriate by those of ordinary skill in the art. For example, the dosage can depend on a number of factors including the requirements of the patient, the severity of the B-cell malignancy (e.g., non-Hodgkin lymphoma or chronic lymphocytic leukemia) associated with the tumor, and the pharmacological activity of the compound being used. The determination of optimum dosages for a particular patient is well known to those skilled in the art.

Various assays and model systems can be readily employed to determine the therapeutic effectiveness of the treatment according to the present technology. Effectiveness of the compositions (as well as determination of effective amounts) and methods of the present technology may also be demonstrated by a decrease in the mass of a tumor and/or slowing the growth of a tumor and/or affecting an increase in the therapeutic responsiveness of a cancer to a second cancer therapy (such as a radiation therapy, a monoclonal antibody, and/or a chemotherapeutic).

For each of the indicated conditions described herein, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

The compounds of the present technology can also be administered to a patient along with other conventional therapeutic agents that may be useful in the treatment of tumors or in vaccination. The administration may include oral administration, parenteral administration, or nasal administration. In any of these embodiments, the administration may include intratumoral injections, subcutaneous injections, intravenous injections, intraperitoneal injections, or intramuscular injections. In any of these embodiments, the administration may include oral administration. The methods of the present technology can also include administering, either sequentially or in combination with one or more compounds of the present technology, a conventional therapeutic agent in an amount that can potentially or synergistically be effective for the treatment a cancer (e.g., a breast cancer, an ovarian cancer, a pancreatic cancer, a biliary tract cancer, a lung cancer, a prostatic cancer, and/or a CNS cancer).

In one aspect, a compound of the present technology is administered to a patient in an amount or dosage suitable for therapeutic use. Generally, a unit dosage comprising a compound of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1×10⁻⁴ g/kg to 1 g/kg, preferably, 1×10⁻³ g/kg to 1.0 g/kg. Dosage of a compound of the present technology can also vary from 0.01 mg/kg to 100 mg/kg or, preferably, from 0.1 mg/kg to 10 mg/kg.

A compound of the present technology can also be modified, for example, by the covalent attachment of an organic moiety or conjugate to improve pharmacokinetic properties, toxicity or bioavailability (e.g., increased in vivo half-life). The conjugate can be a linear or branched hydrophilic polymeric group, fatty acid group or fatty acid ester group. A polymeric group can comprise a molecular weight that can be adjusted by one of ordinary skill in the art to improve, for example, pharmacokinetic properties, toxicity or bioavailability. Exemplary conjugates can include a polyalkane glycol (e.g., polyethylene glycol (PEG), polypropylene glycol (PPG)), carbohydrate polymer, amino acid polymer or polyvinyl pyrolidone and a fatty acid or fatty acid ester group, each of which can independently comprise from about eight to about seventy carbon atoms. Conjugates for use with a compound of the present technology can also serve as linkers to, for example, any suitable substituents or groups, radiolabels (marker or tags), halogens, proteins, enzymes, polypeptides, other therapeutic agents (for example, a pharmaceutical or drug), nucleosides, dyes, oligonucleotides, lipids, phospholipids and/or liposomes. In one aspect, conjugates can include polyethylene amine (PEI), polyglycine, hybrids of PEI and polyglycine, polyethylene glycol (PEG) or methoxypolyethylene glycol (mPEG). A conjugate can also link a compound of the present technology to, for example, a label (fluorescent or luminescent) or marker (radionuclide, radioisotope and/or isotope) to comprise a probe of the present technology. Conjugates for use with a compound of the present technology can, in one aspect, improve in vivo half-life. Other exemplary conjugates for use with a compound of the present technology as well as applications thereof and related techniques include those generally described by U.S. Pat. No. 5,672,662, which is hereby incorporated by reference herein.

In another aspect, the present technology provides methods of identifying a target of interest including contacting the target of interest with a detectable or imaging effective quantity of a labeled compound of the present technology. A detectable or imaging effective quantity is a quantity of a labeled compound of the present technology necessary to be detected by the detection method chosen. For example, a detectable quantity can be an administered amount sufficient to enable detection of binding of the labeled compound to a target of interest. Suitable labels are known by those skilled in the art and can include, for example, radioisotopes, radionuclides, isotopes, fluorescent groups, biotin (in conjunction with streptavidin complexation), and chemiluminescent groups. Upon binding of the labeled compound to the target of interest, the target may be isolated, purified and further characterized such as by determining the amino acid sequence.

The terms “associated” and/or “binding” can mean a chemical or physical interaction, for example, between a compound of the present technology and a target of interest. Examples of associations or interactions include covalent bonds, ionic bonds, hydrophilic-hydrophilic interactions, hydrophobic-hydrophobic interactions and complexes. Associated can also refer generally to “binding” or “affinity” as each can be used to describe various chemical or physical interactions. Measuring binding or affinity is also routine to those skilled in the art. For example, compounds of the present technology can bind to or interact with a target of interest or precursors, portions, fragments and peptides thereof and/or their deposits.

As indicated previously in this disclosure, in an aspect a method of treating a subject suffering from a cancer is provided, where the method includes administering to the subject an effective amount of a compound of any embodiment disclosed herein or administering an effective amount of a composition of any embodiment disclosed herein, and optionally an effective amount of second cancer therapy. In any embodiment herein, the cancer may be a breast cancer, an ovarian cancer, a pancreatic cancer, a biliary tract cancer, a lung cancer, a prostatic cancer, and/or a CNS cancer (such as a brain cancer).

In any embodiment herein, the administering may further include administration of a radiation therapy, a monoclonal antibody, and/or a chemotherapeutic (such as an alkylating agent; a nitrosourea; an antimetabolite; an anthracycline; a topoisomerase II inhibitor; a mitotic inhibitor; an anti-estrogen; a progestin; an aromatase inhibitor; an anti-androgen; an LHIRH agonist; a corticosteroid hormone; a DNA alkylating agent; a taxane; a vinca alkaloid; a microtubule poison, or a combination of any two or more thereof. In any embodiment herein, the administering may further include administration of a chemotherapeutic agent such as busulfan, cisplatin, carboplatin, oxaliplatin, an octahedral platinum (IV) compound, chlorambucil, cyclophosphamide, ifosfamide, dacarbazine (DTIC), mechlorethamine (nitrogen mustard), melphalan, temozolomide, carmustine (BCNU), lomustine (CCNU), 5-fluorouracil, capecitabine, 6-mercaptopurine, methotrexate, gemcitabine, cytarabine (ara-C), fludarabine, pemetrexed, daunorubicin, doxorubicin (Adriamycin), epirubicin, idarubicin, mitoxantrone, topotecan, irinotecan, etoposide (VP-16), teniposide, paclitaxel, docetaxel, vinblastine, vincristine, vinorelbine, prednisone, dexamethasone, L-asparaginase, dactinomycin, thalidomide, tretinoin, imatinib (Gleevec), gefitinib (Iressa), erlotinib (Tarceva), rituximab (Rituxan), bevacizumab (Avastin), ipilimumab, nivolumab (Opdivo), pembrolizumab (Ketruda), tamoxifen, fulvestrant, anastrozole, exemestane, letrozole, megestrol acetate, bicalutamide, flutamide, leuprolide, goserelin, or a combination of any two or more thereof).

In any embodiment herein, the administering may include oral, rectal, nasal, vaginal, parenteral, transdermal, intravenous, intramuscular, or inhalation administration. In any embodiment herein, the administering may include local administration of the compound to a site in the subject including the cancer or local administration of the composition to a site in the subject including the cancer.

The examples herein are provided to illustrate advantages of the present technology and to further assist a person of ordinary skill in the art with preparing or using the compounds and compositions of the present technology. The examples herein are also presented in order to more fully illustrate the preferred aspects of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects, or embodiments of the present technology.

### EXAMPLES

All solvents and reagents were used as received from commercial suppliers, unless noted otherwise. ¹H spectra were recorded on a Bruker AM or Varian 400 spectrometer (operating at 400 MHz respectively) in CDCl₃ with 0.03% TMS as an internal standard. The chemical shifts (S) reported are given in parts per million (ppm) and the coupling constants (J) are in Hertz (Hz). The spin multiplicities are reported as s=singlet, d=doublet, t=triplet, q=quartet, dd=doublet of doublet, ddd=doublet of doublet of doublet, dt=doublet of triplet, td=triplet of doublet, and m=multiplet. The LCMS analysis was performed on an chromatograph with photodiode array UV detection and a TOF mass spectrometer. The mass spectrometer utilized a multimode source which simultaneously acquires ESI+/APCI+; a reference mass solution; and a make-up solvent which was introduced to the LC flow prior to the source to assist ionization. While single stereoisomers were isolated and obtained in high purity where indicated in the below examples, a final unambiguous assignment of absolute stereochemistry may be pending for certain sets of stereoisomers.

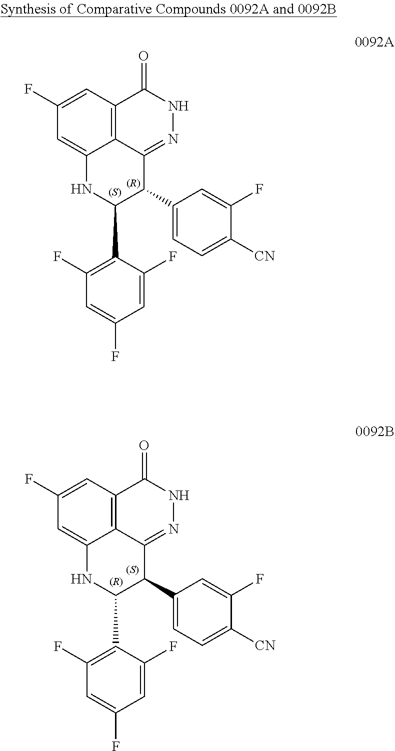

Step 1: To a solution of 6-fluoro-4-nitro-3H-2-benzofuran-1-one (400 mg, 2.03 mmol) in THE (20 mL) were added TEA (575 mg, 5.68 mmol) and Ac₂O (1284 mg, 12.58 mmol) and 2-fluoro-4-formylbenzonitrile (605 mg, 4.06 mmol). The solution was stirred at 80° C. for 3 h. The reaction solution was cooled to room temperature. The mixture was filtered. The filter cake was concentrated in vacuum to obtain crude product (290 mg, 41%) as a yellow solid, which was used directly without further purification. MS (ESI): mass calcd. for C₁₆H₆F₂N₂O₄, 328.0, m/z found 351.0 [M+Na]+

Step 2: To a solution of 2-fluoro-4-{[(1Z)-5-fluoro-7-nitro-3-oxo-2-benzofuran-1-ylidene]methyl}benzonitrile (300 mg, 0.91 mmol) in MeOH (10 mL) was added HCl (4N in MeOH, 10 mL). The solution was stirred at 25° C. for 12 h. The solvent was removed in vacuum. The crude product (300 mg, 87%) was used for next step directly without further purification. MS (ESI): mass calcd. for C₁₇H₁₀F₂N₂O₅, 360.0, m/z found 361.0 [M+H]+

Step 3: To a solution of methyl 2-[2-(4-cyano-3-fluorophenyl)acetyl]-5-fluoro-3-nitrobenzoate (200 mg, 0.56 mmol) and 2,4,6-trifluorobenzaldehyde (166 mg, 1.04 mmol) in THE (30 mL) and MeOH (5 mL) was added TiCl₃ (20% in HCl, 2548 mg, 3.33 mmol). The solution was stirred at 40° C. for 12 h. The mixture was quenched with H₂O (50 mL), extracted with EA (50 mL*3), the combined organic layer was washed with brine (50 mL), then dried over Na₂SO₄ and concentrated in vacuum. The crude product was purified with prep-TLC (PE:EA=3:1) to obtain the desired product (230 mg, 84%) as a white solid. MS (ESI): mass calcd. for C₂₄H₁₃F₅N₂O₃, 472.0, m/z found 473.0 [M+H]+

Step 4: To a solution of methyl 3-(4-cyano-3-fluorophenyl)-7-fluoro-4-oxo-2-(2,4,6-trifluorophenyl)-2,3-dihydro-1H-quinoline-5-carboxylate (100 mg, 0.21 mmol) in MeOH (10 mL) was added N₂H₄·H₂O (37 mg, 0.74 mmol). The solution was stirred at 25° C. for 2 h. The solution was quenched with H₂O (50 mL), extracted with EA (50 mL*3), the combined organic layer was washed with brine (50 mL). Then dried over Na₂SO₄ and concentrated in vacuum. The crude product was purified by flash (CH₃CN:H₂O=1:3) to obtain the desired product (50 mg, 51%) as a white solid. MS (ESI): mass calcd. for C₂₃H₁₁F₅N₄O, 454.0, m/z found 455.0 [M+H]+

Step 5: The 2-fluoro-4-(5-fluoro-3-oxo-8-(2,4,6-trifluorophenyl)-2,7,8,9-tetrahydro-3H-pyrido[4,3,2-de]phthalazin-9-yl)benzonitrile was separated via prep-SFC (Apparatus: SFC 80; Column: Daicel CHIRALCEL OD, 250 mm×30 mm ID, 10 μm; Mobile phase: CO₂/MeOH [0.2% NH₃ (7M Solution in MeOH)]=50/50; Flow rate: 70 g/min; Wave length: UV 214 nm; Temperature: 35° C.) to afford 2-fluoro-4-((8S,9R)-5-fluoro-3-oxo-8-(2,4,6-trifluorophenyl)-2,7,8,9-tetrahydro-3H-pyrido[4,3,2-de]phthalazin-9-yl)benzonitrile (13.1 mg, 51%) as a white solid and 2-fluoro-4-((8R,9S)-5-fluoro-3-oxo-8-(2,4,6-trifluorophenyl)-2,7,8,9-tetrahydro-3H-pyrido[4,3,2-de]phthalazin-9-yl)benzonitrile (12.6 mg, 51%) as a white solid. MS (ESI): mass calcd. for C₂₃H₁₁F₅N₄O, 454.0, m/z found 455.0 [M+H]+. 1H NMR (400 MHz, DMSO-d6) δ 12.33 (s, 1H), 7.89 (s, 1H), 7.81 (t, J=8.0 Hz, 1H), 7.60 (d, J=12.0 Hz, 1H), 7.31 (dd, J=8.0 Hz, 1.2 Hz, 1H), 7.18 (t, J=8.0 Hz, 2H), 7.09 (dd, J=9.2 Hz, 1.6 Hz, 1H), 6.85 (dd, J=10.8 Hz, 2.4 Hz, 1H), 5.28 (d, J=12.0 Hz, 1H), 4.71 (d, J=12.0 Hz, 1H).

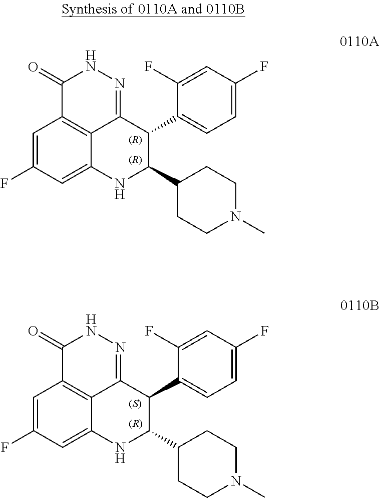

Step 1: To a solution of 6-fluoro-4-nitro-3H-2-benzofuran-1-one (5 g, 25.4 mmol) in THE (80 mL) were added trimethylamine (5.14 g, 50.8 mmol), Ac₂O (15 mL) and 3,4-difluorobenzaldehyde (7.22 g, 50.80 mmol) under N₂. The reaction mixture was stirred at 25° C. for 5 min and then warmed to 80° C. stirred for 3 h. The reaction mixture was quenched with water (100 mL) and extracted with EtOAc (100 mL×3). The combined organic layer washed with brine (100 mL), dried over Na₂SO₄, concentrated to get the residue. The residue was beat with EtOAc (30 mL), filtered and the filter cake was collected and concentrated to get (Z)-3-(2,4-difluorobenzylidene)-6-fluoro-4-nitroisobenzofuran-1(3H)-one (2.2 g, 24%) as a yellow solid.

Step 2: (Z)-3-(2,4-difluorobenzylidene)-6-fluoro-4-nitroisobenzofuran-1(3H)-one (1.0 g, 3.00 mmol) was added to 4 M HCl in MeOH (30 mL). The reaction mixture was stirred at 70° C. for 48 h. The reaction mixture was concentrated to get methyl 2-(2-(2,4-difluorophenyl)acetyl)-5-fluoro-3-nitrobenzoate (1.1 g, 90%) as a yellow solid. MS (ESI): mass calcd. for C₁₆H₁₀F₃NO₅ 353.05, m/z found 354.1 [M+H]⁺.

Step 3: To a solution of methyl 2-(2-(2,4-difluorophenyl)acetyl)-5-fluoro-3-nitrobenzoate (270 mg, 0.76 mmol) and 1-methylpiperidine-4-carbaldehyde (194 mg, 1.50 mmol) in THE (18 mL) and MeOH (3 mL) at 0° C. was added TiCl₃ in HCl (5.25 g, 6.82 mmol) under N₂. The reaction mixture was stirred at 40° C. for 16 h. The reaction mixture was quenched with water (40 mL) and extracted with EtOAc (60 mL×3). The combined organic layer washed with brine (60 mL), dried over Na₂SO₄, concentrated to get the residue. The residue was purified by prep-TLC (MeOH/DCM=1/10, rf=0.4), fraction with MS signal of desired product was collected and concentrated to get methyl 3-(2,4-difluorophenyl)-7-fluoro-2-(1-methylpiperidin-4-yl)-4-oxo-1,2,3,4-tetrahydroquinoline-5-carboxylate (60 mg, 18%) as a yellow solid. MS (ESI): mass calcd. for C₂₃H₂₃F₃N₂O₃ 432.17, m/z found 433.1 [M+H]⁺.

Step 4: A solution of methyl 3-(2,4-difluorophenyl)-7-fluoro-2-(1-methylpiperidin-4-yl)-4-oxo-1,2,3,4-tetrahydroquinoline-5-carboxylate (60 mg, 0.18 mmol) in MeOH (8 mL) and stirred at 25° C. for 20 min, N₂H₄·H₂O (436 mg, 6.96 mmol) was added. The mixture was stirred at 25° C. for 16 h. The reaction mixture was concentrated to get the residue. The residue was purified by prep-HPLC (Chromatographic columns: Xbridge 5 u-C18 150×19 mm, 5 um Mobile Phase: ACN-H₂O (0.1% formic acid), B (Acetonitrile), flow rate: 20 mL/min, wavelength: 214/254 nm, to get 9-(2,4-difluorophenyl)-5-fluoro-8-(1-methylpiperidin-4-yl)-2,7,8,9-tetrahydro-3H-pyrido[4,3,2-de]phthalazin-3-one (40 mg, 50%) as a yellow solid. MS (ESI): mass calcd. for C₂₂H₂₁F₃N₄O 414.17, m/z found 415.1 [M+H]⁺.

Step 5: 9-(2,4-difluorophenyl)-5-fluoro-8-(1-methylpiperidin-4-yl)-2,7,8,9-tetrahydro-3H-pyrido[4,3,2-de]phthalazin-3-one (40 mg, 0.09 mmol) was split via prep-SFC (Daicel CHIRALPAK IB 30*250 mm, 10 um mobile phase A/B: CO₂/MeOH (0.1% EDA))=60/40, flow rate: 1.5 mL/min, column temp: 34 degree) to give (8R,9R)-9-(2,4-difluorophenyl)-5-fluoro-8-(1-methylpiperidin-4-yl)-2,7,8,9-tetrahydro-3H-pyrido[4,3,2-de]phthalazin-3-one (6.7 mg, 17%) as a white solid and (8R,9S)-9-(2,4-difluorophenyl)-5-fluoro-8-(1-methylpiperidin-4-yl)-2,7,8,9-tetrahydro-3H-pyrido[4,3,2-de]phthalazin-3-one (8.2 mg, 20%) as a white solid.

0110A: MS (ESI): mass calcd. for C22H21F3N4O 414.17, m/z found 415.1 [M+H]⁺. ¹H NMR (400 MHz, MeOD) δ=7.15 (dd, J=10.0 Hz, 4.0 Hz, 1H), 7.03-6.98 (m, 1H), 6.85-6.78 (m, 2H), 6.75-6.69 (m, 1H), 4.55 (d, J=3.6 Hz, 1H), 3.37-3.31 (m, 1H), 2.99-2.90 (m, 2H), 2.33 (s, 3H), 2.14-2.03 (m, 2H), 1.81 (d, J=12.0 Hz, 1H), 1.63-1.39 (m, 4H).

0110B: MS (ESI): mass calcd. for C22H21F3N4O 414.17, m/z found 415.1 [M+H]⁺. ¹H NMR (400 MHz, MeOD) δ=7.14 (dd, J=10.0 Hz, 4.0, 1H), 7.02-6.97 (m, 1H), 6.85-6.78 (m, 2H), 6.75-6.69 (m, 1H), 4.55 (d, J=3.6 Hz, 1H), 3.36-3.35 (m, 1H), 2.99-2.90 (m, 2H), 2.27 (s, 3H), 2.06-1.95 (m, 2H), 1.78 (d, J=12.0 Hz, 1H), 1.53-1.41 (m, 4H).

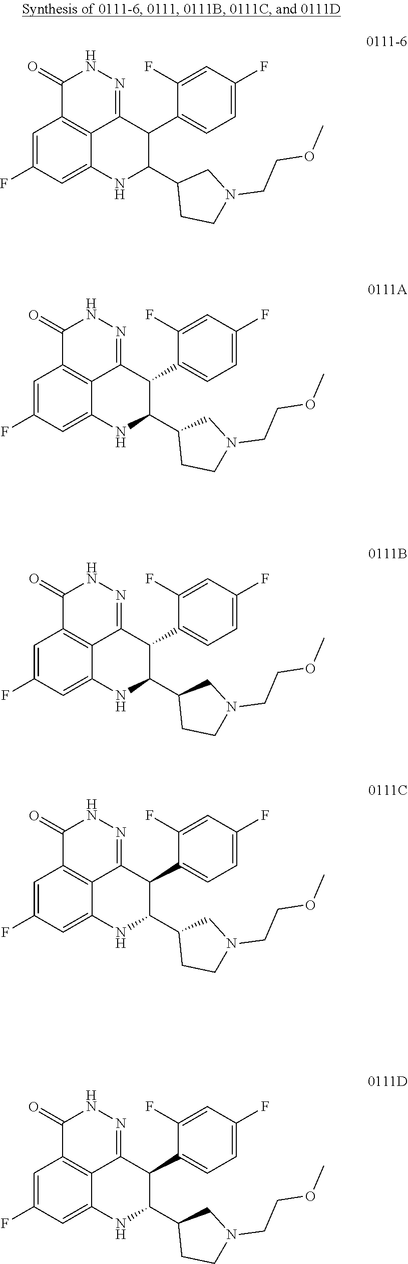 [SMILES: COCCN1CCC(C2Nc3cc(F)cc4c(=O)[nH]nc(c34)C2c2ccc(F)cc2F)C1 ; COCCN1CC[C@H]([C@H]2Nc3cc(F)cc4c(=O)[nH]nc(c34)[C@@H]2c2ccc(F)cc2F)C1 ; COCCN1CC[C@@H]([C@H]2Nc3cc(F)cc4c(=O)[nH]nc(c34)[C@@H]2c2ccc(F)cc2F)C1 ; COCCN1CC[C@H]([C@@H]2Nc3cc(F)cc4c(=O)[nH]nc(c34)[C@H]2c2ccc(F)cc2F)C1 ; COCCN1CC[C@@H]([C@@H]2Nc3cc(F)cc4c(=O)[nH]nc(c34)[C@H]2c2ccc(F)cc2F)C1]

Step1: To a mixture of methyl 2-[2-(2,4-difluorophenyl)acetyl]-5-fluoro-3-nitrobenzoate (1.5 g, 4.20 mmol) and tert-butyl (3-formylpyrrolidin-1-yl) formate (1.7 g, 8.40 mmol) in MeOH (4 mL) and THE (24 mL) was added TiCl₃ (15.0 g, 25.20 mmol). The reaction mixture was stirred at 40° C. for 16 h. The reaction mixture was quenched with H₂O (50 mL) and extracted with EA (2*50 ml). The organic layer was washed with brine (50 mL), dried over Na₂SO₄, filtered, the filtrate was concentrated to give a residue which was purified by reversed phase column (30% A in B; A: CH₃CN, B: 0.1% TFA in water) to afford methyl 3-(2,4-difluorophenyl)-7-fluoro-4-oxo-2-(pyrrolidin-3-yl)-2,3-dihydro-1H-quinoline-5-carboxylate (0.7 g, 38%) as yellow oil. MS (ESI): mass calcd. for C₂₁H₁₉F₃N₂O₃, 404.13, m/z found 405.0 [M+H]⁺.

Step 2: To a solution of methyl 3-(2,4-difluorophenyl)-7-fluoro-4-oxo-2-(pyrrolidin-3-yl)-2,3-dihydro-1H-quinoline-5-carboxylate (950 mg, 2.35 mmol) in ACN (10 mL) were added 1-bromo-2-methoxyethane (327 mg, 2.35 mmol) and K₂CO₃ (974 mg, 7.05 mmol). The reaction mixture was stirred at 80° C. for 16 hours. After cooling to room temperature, the reaction mixture was quenched by H₂O (15 mL), extracted with EA (2*20 mL). The organic layer was washed with brine (20 mL), dried over anhydrous Na₂SO₄, concentrated under vacuum. The residue was purified by flash chromatograph on silica gel column (PE:EA=3:1) to afford methyl 3-(2,4-difluorophenyl)-7-fluoro-2-[1-(2-methoxyethyl)pyrrolidin-3-yl]-4-oxo-2,3-dihydro-1H-quinoline-5-carboxylate (600 mg, 50%) as yellow oil. MS (ESI): mass calcd. C₂₄H₂₅F₃N₂O₄, 462.18, m/z found 463.1 [M+H]⁺.

Step 3: To a solution of methyl 3-(2,4-difluorophenyl)-7-fluoro-2-[1-(2-methoxyethyl)pyrrolidin-3-yl]-4-oxo-2,3-dihydro-1H-quinoline-5-carboxylate (600 mg, 1.30 mmol) in MeOH (10 mL) was added N₂H₄H₂O (650 mg, 12.97 mmol). The reaction mixture was stirred at 25° C. for 16 hours. The reaction mixture was quenched by H₂O (15 mL), extracted with EA (2*20 mL). The organic layer was washed with brine (20 mL), dried over anhydrous Na₂SO₄, concentrated under vacuum. The residue was purified by Prep-HPLC (Instrument: Waters Prep-HPLC, Column: Xbridge prep c18 5 um OBD 19*150 mm/WELCH Xtimate C18 21.2*250 mm 10 um, A water (0.1% formic acid), B Acetonitrile 10-20% B in 8 min, hold at 100% B at for 2 min, back to 5% B with 0.5 min, stop at 13 min, flow rate: 20 ml/min, wavelength: 214/254 nm, to afford 12-(2,4-difluorophenyl)-7-fluoro-11-[1-(2-methoxyethyl)pyrrolidin-3-yl]-2,3,10-triazatricyclo[7.3.1.0\hat{ }{5,13}]trideca-1,5(13),6,8-tetraen-4-one (“0111-6”; 300 mg, 50%) as a yellow solid. MS (ESI): mass calcd. C₂₃H₂₃F₃N₄O₂, 444.18, m/z found 445.1 [M+H]⁺. ¹H NMR (400 MHz, CD₃OD) δ 8.36 (s, 1H), 7.24-7.15 (m, 1H), 7.08-6.96 (m, 1H), 6.91-6.62 (m, 3H), 4.47-4.27 (m, 1H), 3.71-3.57 (m, 4H), 3.45-3.39 (m, 3H), 3.39-3.33 (m, 3H), 3.29-3.23 (m, 1H), 2.75-2.59 (m, 1H), 2.39-1.96 (m, 3H).

Step 4: 0111-6 (300 mg, 0.68 mmol) was separated by Prep-SFC (Apparatus: SFC 80 Column: Daicel CHIRALPAK OX_3, 3*150 mm, 3 um, Mobile phase: A/B: CO₂/MeOH (0.1% DEA)=65/35 Flow rate: 70 g/min Wave length: UV 214 nm Temperature: 35° C.) and (Apparatus: SFC 80 Column: Daicel CHIRALPAK IE_3, 3.0*150 mm, 3 um, Mobile phase: A/B: CO₂/MeOH (0.1% DEA)=65/35 Flow rate: 70 g/min Wave length: UV 214 nm Temperature: 35° C.) to give 0111 (27.2 mg, 9%) as yellow solid, 0111B (20.7 mg, 7%) as white solid, 0111C (48 mg, 16%) as white solid and 0111D (54.9 mg, 18%) as yellow solid.

0111A: ¹H NMR (400 MHz, CDCl₃) δ 9.86 (s, 1H), 7.32-7.27 (m, 1H), 7.00-6.82 (m, 3H), 6.75-6.69 (m, 1H), 6.34 (s, 1H), 4.22 (d, J=8.0 Hz, 1H), 3.60-3.48 (m, 3H), 3.37 (s, 3H), 3.04 (d, J=8.0 Hz, 2H), 2.82-2.72 (m, 1H), 2.70-2.60 (m, 1H), 2.43-2.30 (m, 2H), 2.29-2.20 (m, 1H), 2.11-2.01 (m, 1H), 1.82-1.70 (m, 1H).

0111B: ¹H NMR (400 MHz, CDCl₃) δ 9.74 (s, 1H), 7.31-7.27 (m, 1H), 7.01-6.92 (m, 1H), 6.91-6.83 (m, 2H), 6.77-6.69 (m, 1H), 6.37 (s, 1H), 4.21 (d, J=9.1 Hz, 1H), 3.61-3.49 (m, 3H), 3.37 (s, 3H), 3.14-3.00 (m, 2H), 2.84-2.73 (m, 1H), 2.73-2.63 (m, 1H), 2.42-2.31 (m, 2H), 2.30-2.20 (m, 1H), 2.12-2.02 (m, 1H), 1.83-1.74 (m, 1H).

0111C: ¹H NMR (400 MHz, CDCl₃) δ 9.65 (s, 1H), 7.26-7.22 (m, 2H), 7.16-7.08 (m, 1H), 6.97-6.84 (m, 2H), 6.73-6.63 (m, 1H), 4.13 (d, J=12.0 Hz, 1H), 3.77 (d, J=12.0 Hz, 1H), 3.60-3.52 (m, 2H), 3.43 (s, 3H), 3.27-3.14 (m, 1H), 3.04-2.90 (m, 1H), 2.84-2.74 (m, 1H), 2.69-2.56 (m, 1H), 2.44-2.30 (m, 1H), 2.28-2.17 (m, 1H), 2.10-2.02 (m, 1H), 1.92-1.75 (m, 2H).

0111D: ¹H NMR (400 MHz, CDCl₃) δ 9.70 (s, 1H), 7.26-7.21 (m, 2H), 7.17-7.07 (m, 1H), 7.00-6.77 (m, 2H), 6.65 (d, J=12.0 Hz, 1H), 4.13 (d, J=12.0 Hz, 1H), 3.77 (d, J=12.0 Hz, 1H), 3.60-3.49 (m, 2H), 3.44 (s, 3H), 3.26-3.11 (m, 1H), 3.02-2.88 (m, 1H), 2.85-2.69 (m, 1H), 2.69-2.53 (m, 1H), 2.45-2.27 (m, 1H), 2.25-1.98 (m, 3H), 1.90-1.77 (m, 1H).

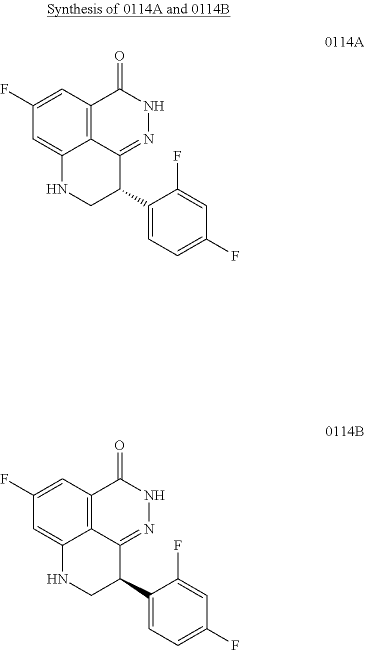 [SMILES: O=c1[nH]nc2c3c(cc(F)cc13)NC[C@H]2c1ccc(F)cc1F ; O=c1[nH]nc2c3c(cc(F)cc13)NC[C@@H]2c1ccc(F)cc1F]

Step 1: To a solution of methyl 2-(2-(2,4-difluorophenyl) acetyl)-5-fluoro-3-nitrobenzoate (2 g, 5.67 mmol) in THF/MeOH=6/1 (14 mL) were added Paraformaldehyde (1.5 g, 17.01 mmol) and TiCl₃ in HCl (14 mL) under N₂, the reaction mixture was stirred at 40° C. for 6 h. The reaction mixture was quenched with water (50 mL) and extracted with EtOAc (50 mL×3), the combined organic layer washed with brine (80 mL), dried over Na₂SO₄, concentrated to get the residue. The residue was purified by column chromatography on silica gel (25 g) eluting with EtOAc in petroleum ether (45%), fraction with MS signal of desired product was collected and concentrated to get methyl 3-(2,4-difluorophenyl)-7-fluoro-4-oxo-1,2,3,4-tetrahydroquinoline-5-carboxylate (150 mg, 7.9%) as a yellow oil. MS (ESI): mass calcd. for C₁₇H₁₂F₃NO₃ 335.08, m/z found 336.1 [M+H]⁺.

Step 2: To a solution of methyl 3-(2,4-difluorophenyl)-7-fluoro-4-oxo-2,3-dihydro-1H-quinoline-5-carboxylate (150 mg, 0.45 mmol) in MeOH (8 mL) was added N₂H₄·H₂O (8 mL) under N₂, the reaction mixture was stirred at 25° C. for 16 h. The reaction mixture was directly concentrated to get the residue. The residue was purified by prep-HPLC (Chromatographic columns: Xbridge 5 u-C18 150×19 mm, 5 um Mobile Phase A: ACN-H₂O (0.1% formic acid), B (Acetonitrile), flow rate: 20 mL/min, wavelength: 214/254 nm, to get 9-(2,4-difluorophenyl)-5-fluoro-2,7,8,9-tetrahydro-3H-pyrido[4,3,2-de]phthalazin-3-one (60 mg, 42%) as a white solid. MS (ESI): mass calcd. for C₁₆H₁₀F₃N₃O 317.08, m/z found 318.1 [M+H]⁺.

Step 3: 9-(2,4-difluorophenyl)-5-fluoro-2,7,8,9-tetrahydro-3H-pyrido[4,3,2-de]phthalazin-3-one (60 mg, 0.19 mmol) was split via prep-SFC (Daicel CHIRALPAK AD_3 3*150 mm, 3 um mobile phase A/B: CO₂/MeOH (0.1% EDA))=65/35, flow rate: 2.0 mL/min, column temp: 37 degree) to give (R)-9-(2,4-difluorophenyl)-5-fluoro-2,7,8,9-tetrahydro-3H-pyrido[4,3,2-de]phthalazin-3-one (24.0 mg, 40%) as a white solid and (S)-9-(2,4-difluorophenyl)-5-fluoro-2,7,8,9-tetrahydro-3H-pyrido[4,3,2-de]phthalazin-3-one (25.6 mg, 43%) as a white solid. MS (ESI): mass calcd. for C₁₆H₁₀F₃N₃O 317.08, m/z found 318.1 [M+H]⁺.

0114A: ¹H NMR (400 MHz, CD₃OD) δ 7.18 (dd, J=9.2, 2.4 Hz, 1H), 7.10-6.94 (m, 2H), 6.94-6.75 (m, 2H), 4.52-4.47 (m, 1H), 3.71-3.57 (m, 2H).

0114B: ¹H NMR (400 MHz, CD₃OD) δ 7.18 (dd, J=9.2, 2.4 Hz, 1H), 7.10-6.95 (m, 2H), 6.93-6.78 (m, 2H), 4.53-4.47 (m, 1H), 3.71-3.56 (m, 2H).

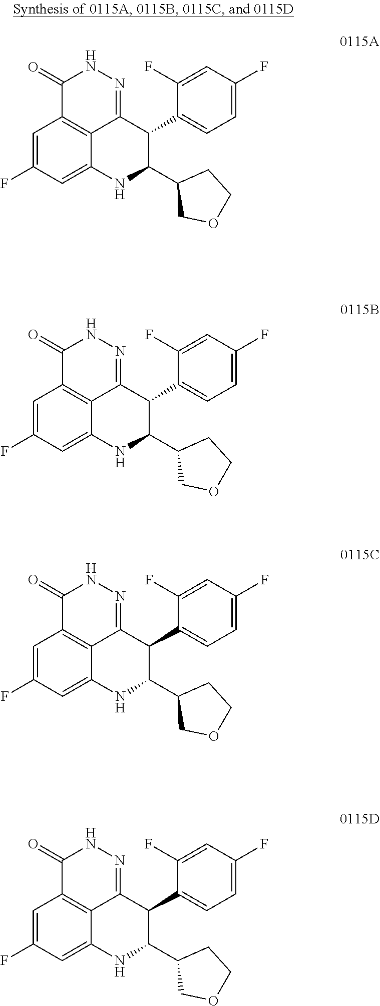 [SMILES: O=c1[nH]nc2c3c(cc(F)cc13)N[C@H]([C@H]1CCOC1)[C@H]2c1ccc(F)cc1F ; O=c1[nH]nc2c3c(cc(F)cc13)N[C@H]([C@@H]1CCOC1)[C@H]2c1ccc(F)cc1F ; O=c1[nH]nc2c3c(cc(F)cc13)N[C@@H]([C@H]1CCOC1)[C@@H]2c1ccc(F)cc1F ; O=c1[nH]nc2c3c(cc(F)cc13)N[C@@H]([C@@H]1CCOC1)[C@@H]2c1ccc(F)cc1F]

Step 1: To a solution of methyl 2-[2-(2,4-difluorophenyl) acetyl]-5-fluoro-3-nitrobenzoate (950 mg, 2.68 mmol) in THE (15 mL), MeOH (3 mL) was added TiCl₃ (12.7 g, 16.13 mmol) at ice bath. The reaction mixture was stirred at 40° C. for 2 h. Then added oxolane-3-carbaldehyde (808 mg, 8.06 mmol) at ice bath. The mixture was stirred at 25° C. for 14 h. The reaction mixture was quenched with H₂O (50 mL) and extracted with EA (2*50 ml). The organic layer was washed with brine (50 mL), dried over Na₂SO₄, filtered, the filtrate was concentrated to give a residue which was purified by silica gel column chromatography eluted with PE:EA (3:1) to afford product compound 3 (200 mg) as a yellow solid, compound 3a (200 mg) as a yellow solid. MS (ESI): mass calcd. for C₂₁H₁₈F₃NO₄, 405.12, m/z found 406.1 [M+H]⁺.

Step 2: To a solution of methyl (2R,3R)-3-(2,4-difluorophenyl)-7-fluoro-4-oxo-2-(oxolan-3-yl)-2,3-dihydro-1H-quinoline-5-carboxylate (200 mg, 0.49 mmol) in MeOH (5 mL) was added NH₂NH₂·H₂O (1.56 g, 24.67 mmol). The reaction mixture was stirred at 25° C. for 3 h. The reaction mixture was quenched by H₂O (15 mL), extracted with EA (2*20 mL). The organic layer was washed with brine (20 mL), dried over anhydrous Na₂SO₄, concentrated under vacuum. The residue was purified by silica gel column chromatography eluted with PE/EA (2:1) to afford product (110 mg). Then was separated by prep-SFC Apparatus: SFC 80, Column: Daicel CHIRALCEL OJ, 250 mm×30 mm I.D., 10 μm, Mobile phase: CO₂/MeOH [0.20% NH₃ (7M Solution in MeOH)]=80/20, Flow rate: 70 g/min, Wave length: UV 214 nm, Temperature: 35° C.) this resulted product 25.3 mg (0115) as a white solid, 24.0 mg (0115B) as a white solid.

0115A: MS (ESI): mass calcd. for C₂₀H₁₆F₃N₃O₂, 387.12, m/z found 388.1 [M+H]⁺. ¹H NMR (400 MHz, MeOD) δ 7.16 (dd, J=8.8, 2.0 Hz, 1H), 7.02-6.97 (m, 1H), 6.90-6.72 (m, 3H), 4.27 (d, J=4.0 Hz, 1H), 3.98-3.83 (m, 2H), 3.75-3.62 (m, 2H), 3.56-3.50 (m, 1H), 2.50-2.39 (m, 1H), 2.05-1.82 (m, 2H).

0115B: MS (ESI): mass calcd. for C₂₀H₁₆F₃N₃O₂, 387.12, m/z found 388.1 [M+H]⁺. ¹H NMR (400 MHz, MeOD) δ 7.16 (dd, J=8.8, 2.0 Hz, 1H), 7.02-6.96 (m, 1H), 6.90-6.72 (m, 3H), 4.27 (d, J=4.0 Hz, 1H), 3.96-3.84 (m, 2H), 3.75-3.62 (m, 2H), 3.56-3.50 (m, 1H), 2.50-2.39 (m, 1H), 2.05-1.82 (m, 2H).

Step 3: To a solution of methyl (2S,3S)-3-(2,4-difluorophenyl)-7-fluoro-4-oxo-2-(oxolan-3-yl)-2,3-dihydro-1H-quinoline-5-carboxylate (200 mg, 0.49 mmol) in MeOH (5 mL) was added NH₂NH₂·H₂O (1.56 g, 24.67 mmol). The reaction mixture was stirred at 25° C. for 3 h. The reaction mixture was quenched by H₂O (15 mL), extracted with EA (2*20 mL). The organic layer was washed with brine (20 mL), dried over anhydrous Na₂SO₄, concentrated under vacuum. The residue was purified by silica gel column chromatography eluted with PE/EA (2:1) to afford product (120 mg). Then was separated by prep-SFC Apparatus: SFC 80, Column: Daicel CHIRALCEL IC, 250 mm×30 mm I.D., 10 μm, Mobile phase: CO₂/MeOH [0.2% NH₃ (7M Solution in MeOH)]=65/35, Flow rate: 70 g/min, Wave length: UV 214 nm, Temperature: 35° C.) this resulted product 35.4 mg (0115C) as a white solid, 30.6 mg (0115D) as a white solid.

0115C: MS (ESI): mass calcd. for C₂₀H₁₆F₃N₃O₂, 387.12, m/z found 388.1 [M+H]⁺. ¹H NMR (400 MHz, MeOD) δ 7.16 (dd, J=9.2, 2.4 Hz, 1H), 7.05-6.95 (m, 1H), 6.85-6.75 (m, 2H), 6.70-6.62 (m, 1H), 4.45 (d, J=3.2 Hz, 1H), 3.99-3.91 (m, 1H), 3.80-3.66 (m, 3H), 3.50-3.44 (m, 1H), 2.54-2.40 (m, 1H), 2.20-2.09 (m, 1H), 1.90-1.78 (m, 1H).

0115D: MS (ESI): mass calcd. for C₂₀H₁₆F₃N₃O₂, 387.12, m/z found 388.1 [M+H]⁺. ¹H NMR (400 MHz, MeOD) δ 7.16 (dd, J=9.2, 2.4 Hz, 1H), 7.05-6.96 (m, 1H), 6.85-6.75 (m, 2H), 6.70-6.62 (m, 1H), 4.45 (d, J=3.2 Hz, 1H), 3.99-3.92 (m, 1H), 3.80-3.66 (m, 3H), 3.50-3.44 (m, 1H), 2.54-2.40 (m, 1H), 2.20-2.09 (m, 1H), 1.90-1.78 (m, 1H).

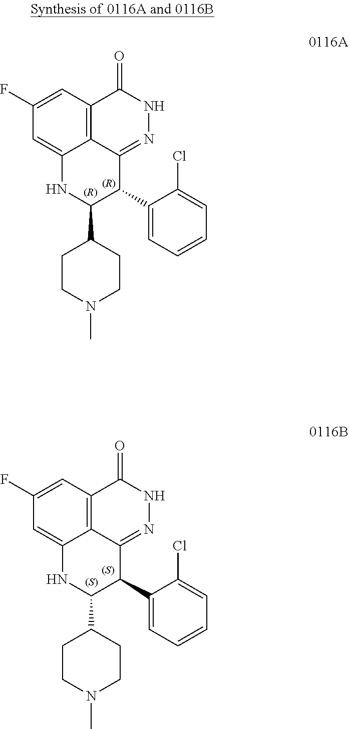

Step 1: To a solution of 6-fluoro-4-nitro-3H-2-benzofuran-1-one (1 g, 5.10 mmol) in THE (20 mL) were added trimethylamine (1.44 g, 14.20 mmol), Ac₂O (3.23 g, 31.60 mmol) and 2-chlorobenzaldehyde (1.43 g, 10.20 mmol) under N₂. The reaction mixture was stirred at 25° C. for 5 min and then warmed to 80° C. stirred for 3 h. The reaction mixture was quenched with water (50 mL) and extracted with EtOAc (80 mL×3), the combined organic layer washed with brine (100 mL), dried over Na₂SO₄, concentrated to get the residue. The residue was beat with EtOAc (30 mL), filtered and the filter cake was collected and concentrated to get (Z)-3-(2-chlorobenzylidene)-6-fluoro-4-nitroisobenzofuran-1(3H)-one (1.1 g, 68%) as a yellow solid. MS (ESI): mass calcd. for C₁₅H₇ClFNO₄ 319.00, m/z found no mass.

Step 2: (3Z)-3-[(2-chlorophenyl)methylidene]-6-fluoro-4-nitro-2-benzofuran-1-one (1.1 g, 3.40 mmol) was added to 4 M HCl in MeOH (30 mL). The reaction mixture was stirred at 70° C. for 48 h. The reaction mixture was concentrated to get methyl 2-(2-(2-chlorophenyl)acetyl)-5-fluoro-3-nitrobenzoate (920 mg, 76%) as a yellow solid. MS (ESI): mass calcd. for C₁₆H₁₁ClFNO₅ 351.03, m/z found 352.0 [M+H]⁺.

Step 3: To a solution of methyl 2-[2-(2-chlorophenyl)acetyl]-5-fluoro-3-nitrobenzoate (400 mg, 1.14 mmol) and 1-methylpiperidine-4-carbaldehyde (289 mg, 2.27 mmol) in THE (18 mL) and MeOH (3 mL) at 0° C. was added TiCl₃ in HCl (5.25 g, 6.82 mmol) under N₂. The reaction mixture was stirred at 40° C. for 16 h. The reaction mixture was quenched with water (20 mL) and extracted with EtOAc (30 mL×3). The combined organic layer washed with brine (30 mL), dried over Na₂SO₄, concentrated to get the residue. The residue was purified by prep-TLC (MeOH/DCM=1/10, rf=0.4), fraction with MS signal of desired product was collected and concentrated to get methyl 3-(2-chlorophenyl)-7-fluoro-2-(1-methylpiperidin-4-yl)-4-oxo-1,2,3,4-tetrahydroquinoline-5-carboxylate (120 mg, 24%) as a yellow oil. MS (ESI): mass calcd. for C₂₃H₂₄ClFN₂O₃ 430.15, m/z found 431.1 [M+H]⁺.

Step 4: A solution of methyl 3-(2-chlorophenyl)-7-fluoro-2-(1-methylpiperidin-4-yl)-4-oxo-2,3-dihydro-1H-quinoline-5-carboxylate (100 mg, 0.23 mmol) in MeOH (8 mL) and stirred at 25° C. for 20 min, N₂H₄·H₂O (436 mg, 6.96 mmol) was added and stirred at 25° C. for 16 h. The reaction mixture was concentrated to get the residue. The residue was purified by prep-HPLC (Chromatographic columns: Xbridge 5 u-C18 150×19 mm, 5 um Mobile Phase: ACN-H₂O (0.1% formic acid), B (Acetonitrile), flow rate: 20 mL/min, wavelength: 214/254 nm, to get 9-(2-chlorophenyl)-5-fluoro-8-(1-methylpiperidin-4-yl)-2,7,8,9-tetrahydro-3H-pyrido[4,3,2-de]phthalazin-3-one (50 mg, 52%) as a white solid. MS (ESI): mass calcd. for C₂₂H₂₂ClFN₄O 412.15, m/z found 413.1 [M+Na]⁺.

Step 5: 9-(2-chlorophenyl)-5-fluoro-8-(1-methylpiperidin-4-yl)-2,7,8,9-tetrahydro-3H-pyrido[4,3,2-de]phthalazin-3-one (50 mg, 0.12 mmol) was split via prep-SFC (Daicel CHIRALPAK IB 30*250 mm, 10 um mobile phase A/B: CO₂/MeOH (0.1% EDA))=60/40, flow rate: 1.5 mL/min, column temp: 34 degree) to give (8R,9R)-9-(2-chlorophenyl)-5-fluoro-8-(1-methylpiperidin-4-yl)-2,7,8,9-tetrahydro-3H-pyrido[4,3,2-de]phthalazin-3-one (15.0 mg, 30%) as a white solid and (8S,9S)-9-(2-chlorophenyl)-5-fluoro-8-(1-methylpiperidin-4-yl)-2,7,8,9-tetrahydro-3H-pyrido[4,3,2-de]phthalazin-3-one (8.5 mg, 17%) as a white solid.

0116A: MS (ESI): mass calcd. for C₂₂H₂₂ClFN₄O 412.15, m/z found 413.1 [M+Na]⁺. ¹H NMR (400 MHz, DMSO-d6) δ 12.44 (s, 1H), 7.50 (dd, J=8.0, 1.2 Hz, 1H), 7.40 (d, J=2.4 Hz, 1H), 7.26 (td, J=7.6, 1.6 Hz, 1H), 7.18 (td, J=7.6, 1.2 Hz, 1H), 7.01 (dd, J=9.2, 2.4 Hz, 1H), 6.90 (dd, J=11.2, 2.4 Hz, 1H), 6.66 (dd, J=7.6, 1.2 Hz, 1H), 4.56 (d, J=3.6 Hz, 1H), 3.36-3.30 (m, 1H), 2.95-2.80 (m, 2H), 2.20 (s, 3H), 1.96 (t, J=10.4 Hz, 1H), 1.91-1.75 (m, 2H), 1.72-1.50 (m, 2H), 1.45-1.33 (m, 2H).

0116B: MS (ESI): mass calcd. for C₂₂H₂₂ClFN₄O 412.15, m/z found 413.1 [M+Na]⁺. H NMR (400 MHz, DMSO-d6) δ 12.42 (s, 1H), 7.49 (dd, J=8.0, 1.2 Hz, 1H), 7.36 (d, J=2.4 Hz, 1H), 7.26 (td, J=7.6, 1.6 Hz, 1H), 7.23-7.11 (m, 1H), 7.00 (dd, J=8.8, 2.4 Hz, 1H), 6.95-6.87 (m, 1H), 6.70-6.66 (m, 1H), 4.55 (d, J=3.6 Hz, 1H), 3.34-3.32 (m, 1H), 2.84-2.68 (m, 2H), 2.08 (s, 3H), 1.72 (t, J=10.4 Hz, 2H), 1.67-1.58 (m, 2H), 1.57-1.46 (m, 1H), 1.40-1.26 (m, 2H).

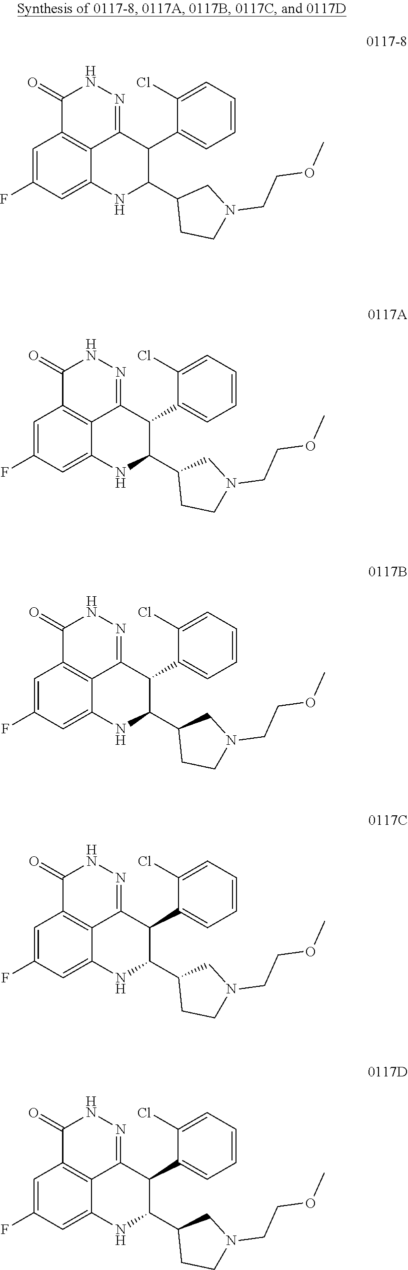 [SMILES: COCCN1CCC(C2Nc3cc(F)cc4c(=O)[nH]nc(c34)C2c2ccccc2Cl)C1 ; COCCN1CC[C@H]([C@H]2Nc3cc(F)cc4c(=O)[nH]nc(c34)[C@@H]2c2ccccc2Cl)C1 ; COCCN1CC[C@@H]([C@H]2Nc3cc(F)cc4c(=O)[nH]nc(c34)[C@@H]2c2ccccc2Cl)C1 ; COCCN1CC[C@H]([C@@H]2Nc3cc(F)cc4c(=O)[nH]nc(c34)[C@H]2c2ccccc2Cl)C1 ; COCCN1CC[C@@H]([C@@H]2Nc3cc(F)cc4c(=O)[nH]nc(c34)[C@H]2c2ccccc2Cl)C1]

Step 1: To a mixture of methyl 2-[2-(2-chlorophenyl)acetyl]-5-fluoro-3-nitrobenzoate (1.0 g, 2.80 mmol) and tert-butyl (3-formylpyrrolidin-1-yl) formate (1.1 g, 5.60 mmol) in THE (24 mL) and MeOH (4 mL) was added TiCl₃ (10.0 g, 84.00 mmol). The reaction mixture was quenched with H₂O (50 mL) and extracted with EA (2*50 ml). The organic layer was washed with brine (50 mL), dried over Na₂SO₄, filtered, the filtrate was concentrated to give a residue which was purified by reversed phase column (30% A in B; A: CH₃CN, B: 0.1% TFA in water) to afford methyl 3-(2-chlorophenyl)-7-fluoro-4-oxo-2-(pyrrolidin-3-yl)-2,3-dihydro-1H-quinoline-5-carboxylate (600 mg, 52%) as yellow oil. MS (ESI): mass calcd. for C₂₁H₂₀ClFN₂O₃, 402.11, m/z found 403.0 [M+H]⁺.

Step 2: To a solution of methyl 3-(2-chlorophenyl)-7-fluoro-4-oxo-2-(pyrrolidin-3-yl)-2,3-dihydro-1H-quinoline-5-carboxylate (600 mg, 1.49 mmol) in ACN (10 mL) were added 1-bromo-2-methoxyethane (207 mg, 1.49 mmol) and K₂CO₃ (618 mg, 4.47 mmol). The reaction mixture was stirred at 80° C. for 16 hours. After cooling to room temperature, the reaction mixture was quenched by H₂O (15 mL), extracted with EA (2*20 mL). The organic layer was washed with brine (20 mL), dried over anhydrous Na₂SO₄, concentrated under vacuum. The residue was purified by flash chromatograph on silica gel column (EA) to afford methyl 3-(2-chlorophenyl)-7-fluoro-2-[1-(2-methoxyethyl)pyrrolidin-3-yl]-4-oxo-2,3-dihydro-1H-quinoline-5-carboxylate (400 mg, 55%) as yellow oil. MS (ESI): mass calcd. C₂₄H₂₆ClFN₂O₄, 460.16, m/z found 461.1 [M+H]⁺.

Step 3: To a solution of methyl 3-(2-chlorophenyl)-7-fluoro-2-[1-(2-methoxyethyl)pyrrolidin-3-yl]-4-oxo-2,3-dihydro-1H-quinoline-5-carboxylate (400 mg, 0.87 mmol) in MeOH (5 mL) was added N₂H₄H₂O (217 mg, 4.34 mmol). The reaction mixture was stirred at 25° C. for 16 hours. The reaction mixture was quenched by H₂O (15 mL), extracted with EA (2*20 mL). The organic layer was washed with brine (20 mL), dried over anhydrous Na₂SO₄, concentrated under vacuum. The residue was purified by Prep-HPLC (Instrument: Waters Prep-HPLC, Column: Xbridge prep c18 5 um OBD 19*150 mm, A water (0.1% formic acid), B Acetonitrile 10-20% B in 8 min, hold at 100% B at for 2 min, back to 5% B with 0.5 min, stop at 13 min., flow rate: 20 ml/min, wavelength: 214/254 nm, to afford 12-(2-chlorophenyl)-7-fluoro-11-[1-(2-methoxyethyl)pyrrolidin-3-yl]-2,3,10-triazatricyclo[7.3.1.0\hat{ }{5,13}]trideca-1,5(13),6,8-tetraen-4-one (“0117-8”; 260 mg, 65%) as a yellow solid. MS (ESI): mass calcd. C₂₃H₂₄ClFN₄O₂, 442.16, m/z found 443.1 [M+H]⁺. ¹H NMR (400 MHz, CDCl₃) δ 9.91 (d, J=80.0 Hz, 1H), 8.56 (s, OH), 7.48-7.40 (m, 1H), 7.32-7.27 (m, 1H), 7.25-7.08 (m, 2H), 6.92-6.79 (m, 1H), 6.73-6.62 (m, 1H), 4.57-4.43 (m, 1H), 3.88-3.53 (m, 3H), 3.43-3.35 (m, 3H), 3.29-2.97 (m, 2H), 2.96-2.49 (m, 3H), 2.45-1.85 (m, 4H).

Step 4: 0117-8 (300 mg, 0.68 mmol) was separated by Prep-SFC (Apparatus: SFC 80 Column: Daicel CHIRALPAK OX_3, 3*150 mm, 3 um, Mobile phase: A/B: CO₂/MeOH (0.1% DEA)=65/35 Flow rate: 70 g/min Wave length: UV 214 nm Temperature: 35° C.) and (Apparatus: SFC 80 Column: Daicel CHIRALPAK IE_3, 3.0*150 mm, 3 um, Mobile phase: A/B: CO₂/MeOH (0.1% DEA)=50/50 Flow rate: 70 g/min Wave length: UV 214 nm Temperature: 35° C.) to give 0117 (13.6 mg, 18%) as white solid, 0117B (16.5 mg, 22%) as white solid, 0117C (26.0 mg, 35%) as white solid and 0117D (28 mg, 37%) as yellow solid. MS (ESI): mass calcd. for C₂₃H₂₄ClFN₄O₂, 442.16, m/z found 443.1 [M+H]⁺.

0117A: ¹H NMR (400 MHz, CD₃OD) δ 7.45-7.36 (m, 1H), 7.22-7.02 (m, 3H), 6.82-6.71 (m, 1H), 6.52 (d, J=7.6 Hz, 1H), 4.57 (d, J=2.4 Hz, 1H), 3.55-3.42 (m, 3H), 3.33-3.28 (m, 3H), 2.95-2.61 (m, 6H), 2.54-2.38 (m, 1H), 2.19-1.91 (m, 2H).

0117B: ¹H NMR (400 MHz, CD₃OD) δ 7.50-7.40 (m, 1H), 7.29-7.17 (m, 2H), 7.14-7.03 (m, 1H), 6.91-6.78 (m, 1H), 6.58-6.48 (m, 1H), 4.64-4.59 (m, 1H), 3.68-3.54 (m, 3H), 3.42-3.31 (m, 4H), 3.29-3.06 (m, 5H), 2.73-2.59 (m, 1H), 2.35-2.23 (m, 1H), 2.22-2.09 (m, 1H).

0117C: ¹H NMR (400 MHz, CD₃OD) δ 7.49-7.37 (m, 1H), 7.26-7.06 (m, 3H), 6.85-6.75 (m, 1H), 6.70-7.58 (m, 1H), 4.51-4.47 (m, 1H), 3.60-3.48 (m, 3H), 3.34 (s, 3H), 2.99-2.58 (m, 6H), 2.49-2.28 (m, 1H), 1.95-1.76 (m, 2H).

0117D: ¹H NMR (400 MHz, CDCl₃) δ 9.76 (s, 1H), 7.54-7.40 (m, 1H), 7.32-7.28 (m, 2H), 7.26 (d, J=2.4 Hz, 1H), 7.13-7.01 (m, 1H), 6.96-6.90 (m, 1H), 4.48 (d, J=10.8 Hz, 1H), 3.91 (d, J=10.4 Hz, 1H), 3.69-3.63 (m, 2H), 3.60-3.49 (m, 2H), 3.43-3.35 (m, 3H), 3.06-3.00 (m, 1H), 2.96-2.90 (m, 1H), 2.84-2.76 (m, 1H), 2.64-2.54 (m, 1H), 2.40-2.24 (m, 2H), 2.12-2.00 (m, 1H).

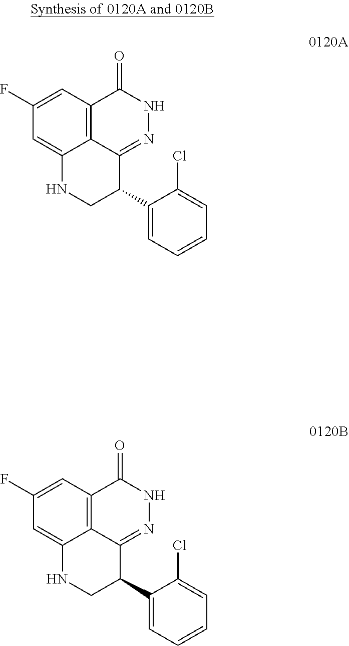 [SMILES: O=c1[nH]nc2c3c(cc(F)cc13)NC[C@H]2c1ccccc1Cl ; O=c1[nH]nc2c3c(cc(F)cc13)NC[C@@H]2c1ccccc1Cl]

Step 1: To a solution of methyl 2-[2-(2-chlorophenyl)acetyl]-5-fluoro-3-nitrobenzoate (1 g, 2.80 mmol) and Paraformaldehyde (1.01 g, 11.20 mmol) in THE (6 mL) and MeOH (1 mL) cooled to 0° C. was added TiCl₃ in HCl (7 mL) under N₂. The reaction mixture was stirred at 40° C. for 2 h. The reaction mixture was quenched with water (20 mL) and extracted with EtOAc (30 mL×3). The combined organic layer was washed with brine (30 mL), dried over Na₂SO₄, concentrated to get the residue. The residue was purified by column chromatography on silica gel (12 g) eluting with EtOAc in petroleum ether (19%), fraction with MS signal of desired product was collected and concentrated to get methyl 3-(2-chlorophenyl)-7-fluoro-4-oxo-1,2,3,4-tetrahydroquinoline-5-carboxylate (120 mg, 13%) as a yellow oil. MS (ESI): mass calcd. for C₁₇H₁₃ClFNO₃ 333.06, m/z found 334.2, 336.2 [M+H]⁺.

Step 2: The mixture of methyl 3-(2-chlorophenyl)-7-fluoro-4-oxo-2,3-dihydro-1H-quinoline-5-carboxylate (140 mg, 0.42 mmol) and N₂H₄·H₂O/MeOH=1/1 (8 mL) was stirred at 25° C. for 16 h. The reaction mixture was concentrated to get the residue. The residue was purified by prep-HPLC (Chromatographic columns: Xbridge 5 u-C18 150×19 mm, 5 um Mobile Phase A: ACN-H₂O (0.1% formic acid), B (Acetonitrile), flow rate: 20 mL/min, wavelength: 214/254 nm, to get 9-(2-chlorophenyl)-5-fluoro-2,7,8,9-tetrahydro-3H-pyrido[4,3,2-de]phthalazin-3-one (50 mg, 38%) as a white solid. MS (ESI): mass calcd. for C₁₆H₁₁ClFN₃O 315.06, m/z found 316.0, 318.0 [M+H]⁺.

Step 3: 9-(2-chlorophenyl)-5-fluoro-2,7,8,9-tetrahydro-3H-pyrido[4,3,2-de]phthalazin-3-one (50 mg, 0.16 mmol) was split via prep-SFC (Daicel CHIRALPAK IB 30*250 mm, 10 um mobile phase A/B: CO₂/MeOH (0.1% EDA))=50/50, flow rate: 1.5 mL/min, column temp: 37 degree) to give (R)-9-(2-chlorophenyl)-5-fluoro-2,7,8,9-tetrahydro-3H-pyrido[4,3,2-de]phthalazin-3-one (14.6 mg, 29%) as a white solid and (S)-9-(2-chlorophenyl)-5-fluoro-2,7,8,9-tetrahydro-3H-pyrido[4,3,2-de]phthalazin-3-one (13.2 mg, 26%) as a white solid.

0120A: MS (ESI): mass calcd. for C₁₆H₁₁ClFN₃O 315.06, m/z found 316.0, 318.0 [M+H]⁺. ¹H NMR (400 MHz, CDCl₃) δ 9.92 (s, 1H), 7.48-7.37 (m, 2H), 7.25-7.13 (m, 2H), 6.88 (dd, J=7.6, 1.6 Hz, 1H), 6.70 (dd, J=10.4, 2.4 Hz, 1H), 4.81-4.71 (m, 2H), 3.76-3.63 (m, 2H).

0120B: MS (ESI): mass calcd. for C₁₆H₁₁ClFN₃O 315.06, m/z found 316.0, 318.0 [M+H]⁺. ¹H NMR (400 MHz, CDCl₃) δ 10.08 (s, 1H), 7.49-7.34 (m, 2H), 7.25-7.11 (m, 2H), 6.87 (dd, J=7.6, 1.6 Hz, 1H), 6.70 (dd, J=10.4, 2.4 Hz, 1H), 4.84-4.75 (m, 2H), 3.86-3.51 (m, 2H).

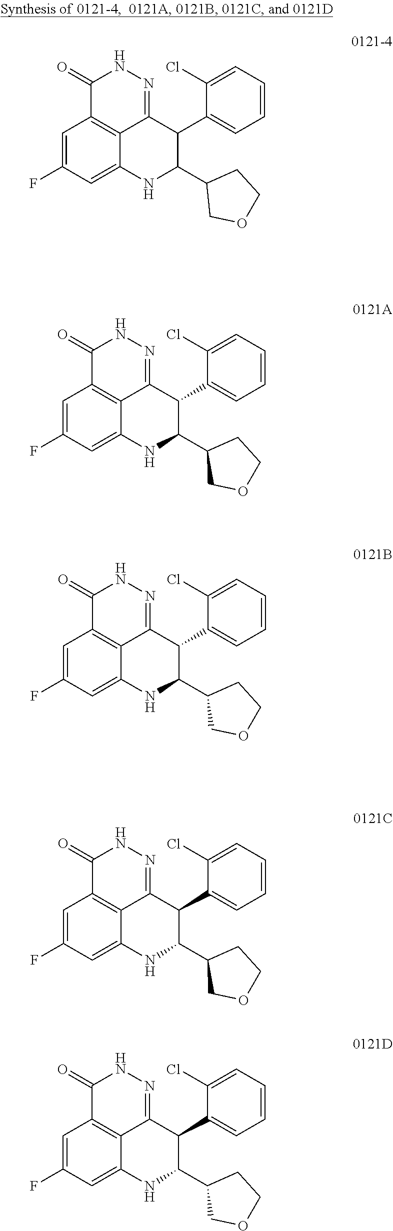 [SMILES: O=c1[nH]nc2c3c(cc(F)cc13)NC(C1CCOC1)C2c1ccccc1Cl ; O=c1[nH]nc2c3c(cc(F)cc13)N[C@H]([C@H]1CCOC1)[C@H]2c1ccccc1Cl ; O=c1[nH]nc2c3c(cc(F)cc13)N[C@H]([C@@H]1CCOC1)[C@H]2c1ccccc1Cl ; O=c1[nH]nc2c3c(cc(F)cc13)N[C@@H]([C@H]1CCOC1)[C@@H]2c1ccccc1Cl ; O=c1[nH]nc2c3c(cc(F)cc13)N[C@@H]([C@@H]1CCOC1)[C@@H]2c1ccccc1Cl]

Step 1: To a solution of methyl 2-[2-(2-chlorophenyl) acetyl]-5-fluoro-3-nitrobenzoate (950 mg, 2.70 mmol) in THE (10 mL), MeOH (2 mL) was added TiCl₃ (12.8 g, 16.20 mmol) at ice bath. The reaction mixture was stirred at 40° C. for 2 h. Then added oxolane-3-carbaldehyde (811 mg, 8.10 mmol) at ice bath. The mixture was stirred at 25° C. for 14 h. The reaction mixture was quenched with H₂O (50 mL) and extracted with EA (2*50 ml). The organic layer was washed with brine (50 mL), dried over Na₂SO₄, filtered, the filtrate was concentrated to give a residue which was purified by silica gel column chromatography eluted with PE:EA (3:1) to afford product (250 mg) as a yellow solid. MS (ESI): mass calcd. for C₂₁H₁₉ClFNO₄, 403.10, m/z found 404.1[M+H]⁺.

Step 2: To a solution of methyl 3-(2-chlorophenyl)-7-fluoro-4-oxo-2-(oxolan-3-yl)-2,3-dihydro-1H-quinoline-5-carboxylate (250 mg, 0.6191 mmol) in MeOH (5 mL) was added NH₂NH₂·H₂O (1.56 g, 24.76 mmol). The reaction mixture was stirred at 25° C. for 3 h. The reaction mixture was quenched by H₂O (15 mL), extracted with EA (2*20 mL). The organic layer was washed with brine (20 mL), dried over anhydrous Na₂SO₄, concentrated under vacuum. The residue was purified by silica gel column chromatography eluted with PE/EA (2:1) to afford product (170 mg). Then taken 20 mg product purified by Pre-HPLC, (Instrument: Waters MS-triggered Prep-LC with QDA detector, Column: Xbridge 5 u C18 150×19 mm, A water (0.1% formic acid), B Acetonitrile 25-55% B in 8 min, hold at 100% B for 2 min, back to 25% B with 0.5 min, stop at 13 min, flow rate: 20 mL/min, wavelength: 214/254 nm, to afford the target compound (“0121-4”; 2.5 mg, 12%) as a white solid.

0121-4: MS (ESI): mass calcd. for C₂₀H₁₇ClFN₃O₂, 385.10, m/z found 386.0 [M+H]⁺. ¹H NMR (400 MHz, MeOD) δ 7.48-7.42 (m, 1H), 7.26-7.05 (m, 3H), 6.90-6.75 (m, 1H), 6.64-6.50 (m, 1H), 4.70-4.45 (m, 1H), 4.01-3.81 (m, 2H), 3.78-3.64 (m, 2H), 3.57-3.46 (m, 1H), 2.58-2.43 (m, 1H), 2.25-1.83 (m, 2H).

Step 3: The product was separated by prep-SFC Apparatus: SFC 80, Column: Daicel CHIRALCEL IC, 250 mm 30 mm I.D., 10 μm, Mobile phase: CO₂/MeOH [0.2% NH₃ (7M Solution in MeOH)]=60/40, Flow rate: 70 g/min, Wave length: UV 214 nm, Temperature: 35° C.) this resulted product 35.8 mg (0121) as a white solid, 31.5 mg (0121B) as a white solid, 19.9 mg (0121C) as a white solid, 26.3 mg (0121D) as a white solid.

0121A: MS (ESI): mass calcd. for C₂₀H₁₇ClFN₃O₂, 385.10, m/z found 386.0 [M+H]⁺. ¹H NMR (400 MHz, MeOD) δ 7.37-7.33 (m, 1H), 7.15-6.98 (m, 3H), 6.78-6.71 (m, 1H), 6.54-6.48 (m, 1H), 4.38 (d, J=3.2 Hz, 1H), 3.87-3.72 (m, 3H), 3.61-3.53 (m, 1H), 3.46-3.40 (m, 1H), 2.46-2.33 (m, 1H), 1.93-1.83 (m, 1H), 1.82-1.70 (m, 1H).

0121B: MS (ESI): mass calcd. for C₂₀H₁₇ClFN₃O₂, 385.10, m/z found 386.0 [M+H]⁺. ¹H NMR (400 MHz, MeOD) δ 7.38-7.33 (m, 1H), 7.15-6.98 (m, 3H), 6.78-6.71 (m, 1H), 6.54-6.48 (m, 1H), 4.38 (d, J=3.2 Hz, 1H), 3.87-3.72 (m, 3H), 3.61-3.53 (m, 1H), 3.46-3.40 (m, 1H), 2.46-2.33 (m, 1H), 1.94-1.83 (m, 1H), 1.82-1.71 (m, 1H).

0121C: MS (ESI): mass calcd. for C₂₀H₁₇ClFN₃O₂, 385.10, m/z found 386.0 [M+H]⁺. ¹H NMR (400 MHz, MeOD) δ 7.42-7.30 (m, 1H), 7.16-6.94 (m, 3H), 6.74-6.66 (m, 1H), 6.49-6.39 (m, 1H), 4.57 (d, J=2.0 Hz, 1H), 3.92-3.82 (m, 1H), 3.71-3.54 (m, 3H), 3.46-3.36 (m, 1H), 2.47-2.32 (m, 1H), 2.15-2.01 (m, 1H), 2.00-1.86 (m, 1H).

0121D: MS (ESI): mass calcd. for C₂₀H₁₇ClFN₃O₂, 385.10, m/z found 386.0 [M+H]⁺. ¹H NMR (400 MHz, MeOD) δ 7.39-7.33 (m, 1H), 7.16-6.95 (m, 3H), 6.75-6.65 (m, 1H), 6.49-6.39 (m, 1H), 4.57 (d, J=2.0 Hz, 1H), 3.92-3.82 (m, 1H), 3.71-3.54 (m, 3H), 3.46-3.36 (m, 1H), 2.47-2.32 (m, 1H), 2.15-2.01 (m, 1H), 2.00-1.86 (m, 1H).

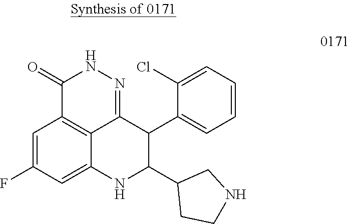 [SMILES: O=c1[nH]nc2c3c(cc(F)cc13)NC(C1CCNC1)C2c1ccccc1Cl]

To a solution of methyl 3-(2-chlorophenyl)-7-fluoro-4-oxo-2-(pyrrolidin-3-yl)-2,3-dihydro-1H-quinoline-5-carboxylate (20 mg, 0.05 mmol) in MeOH (1 mL) were added N₂H₄H₂O (25 mg, 0.50 mmol). The reaction mixture was stirred at 25° C. for 16 hours. The mixture was concentrated and the residue was purified by Prep-HPLC (Instrument: Waters Prep-HPLC, Column: Xbridge prep c18 5 um OBD 19*150 mm, A water (0.1% formic acid), B Acetonitrile 10-20% B in 8 min, hold at 100% B at for 2 min, back to 5% B with 0.5 min, stop at 13 min, flow rate: 20 ml/min, wavelength: 214/254 nm, to afford 12-(2-chlorophenyl)-7-fluoro-11-(pyrrolidin-3-yl)-2,3,10-triazatricyclo[7.3.1.0\hat{ }{5,13}]trideca-1,5(13),6,8-tetraen-4-one (3 mg, 15%) as a yellow solid. MS (ESI): mass calcd. for C₂₀H₁₈ClFN₄O, 384.12, m/z found 385.0 [M+H]⁺. ¹H NMR (400 MHz, CD₃OD) δ 7.55-7.40 (m, 1H), 7.32-7.16 (m, 2H), 7.16-7.11 (m, 1H), 6.93-6.80 (m, 1H), 6.60-6.52 (m, 1H), 4.68-4.50 (m, 1H), 3.70-3.33 (m, 3H), 3.29-3.08 (m, 2H), 2.74-2.55 (m, 1H), 2.43-1.77 (m, 2H).

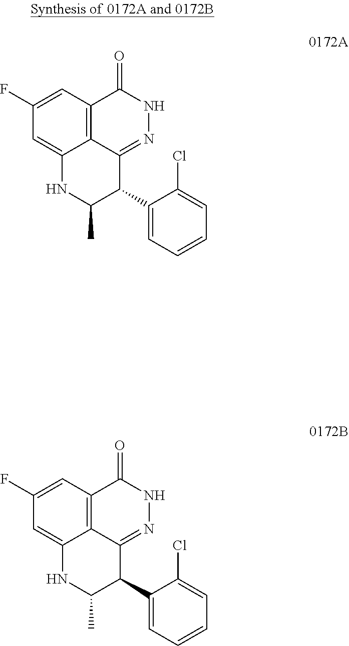 [SMILES: C[C@H]1Nc2cc(F)cc3c(=O)[nH]nc(c23)[C@@H]1c1ccccc1Cl ; C[C@@H]1Nc2cc(F)cc3c(=O)[nH]nc(c23)[C@H]1c1ccccc1Cl]

Step 1: To a solution of methyl 2-[2-(2-chlorophenyl)acetyl]-5-fluoro-3-nitrobenzoate (0.2 g, 0.6 mmol) and Acetaldehyde (0.11 g, 2.40 mmol) in THE (4 mL) and MeOH (0.6 mL) was added TiCl₃ in HCl (8 mL) at 0° C. under N₂. The reaction mixture was stirred at 40° C. for 1 h. The reaction mixture was quenched with water (20 mL) and extracted with EtOAc (30 mL×3). The combined organic layer washed with brine (30 mL), dried over Na₂SO₄, concentrated to get the residue. The residue was purified by column chromatography on silica gel (12 g) eluting with EtOAc in petroleum ether (28%), fraction with MS signal of desired product was collected and concentrated to get methyl 3-(2-chlorophenyl)-7-fluoro-2-methyl-4-oxo-1,2,3,4-tetrahydroquinoline-5-carboxylate (90 mg, 95%) as a colorless oil. MS (ESI): mass calcd. for C₁₈H₁₅ClFNO₃ 347.07, m/z found 348.2, 350.2 [M+H]⁺.

Step 2: A solution of methyl 3-(2-chlorophenyl)-7-fluoro-2-methyl-4-oxo-2,3-dihydro-1H-quinoline-5-carboxylate (100 mg, 0.29 mmol) in MeOH (8 mL) and stirred at 25° C. for 20 min. N₂H₄·H₂O (540 mg, 8.63 mmol) was added and the mixture was stirred at 25° C. for 16 h. The reaction mixture was directly concentrated to get the residue. The residue was purified by prep-HPLC (Chromatographic columns: Xbridge 5 u-C18 150×19 mm, 5 um Mobile Phase A: ACN-H₂O (0.1% formic acid), B (Acetonitrile), flow rate: 20 mL/min, wavelength: 214/254 nm, to get 9-(2-chlorophenyl)-5-fluoro-2,7,8,9-tetrahydro-3H-pyrido[4,3,2-de]phthalazin-3-one (50 mg, 38%) as a white solid. MS (ESI): mass calcd. for C₁₇H₁₃ClFN₃O 329.07, m/z found 330.1, 332.1 [M+H]⁺.

Step 3: 9-(2-chlorophenyl)-5-fluoro-2,7,8,9-tetrahydro-3H-pyrido[4,3,2-de]phthalazin-3-one (90 mg, 0.27 mmol) was split via prep-SFC (Daicel CHIRALPAK IB 30*250 mm, 10 um mobile phase A/B: CO₂/MeOH (0.1% EDA))=65/35, flow rate: 2.0 mL/min, column temp: 37 degree) to give (8R,9R)-9-(2-chlorophenyl)-5-fluoro-8-methyl-2,7,8,9-tetrahydro-3H-pyrido[4,3,2-de]phthalazin-3-one (27.4 mg, 30%) as a white solid and (8S,9S)-9-(2-chlorophenyl)-5-fluoro-8-methyl-2,7,8,9-tetrahydro-3H-pyrido[4,3,2-de]phthalazin-3-one (60.4 mg, 67%) as a white solid.

0172A and 0172B: MS (ESI): mass calcd. for C₁₇H₁₃ClFN₃O 329.07, m/z found 330.0, 332.0 [M+H]⁺. ¹H NMR (400 MHz, CDCl₃) δ 9.74 (s, 1H), 7.47-7.45 (m, 1H), 7.38-7.35 (m, 1H), 7.26-7.22 (m, 2H), 7.01-6.99 (m, 1H), 6.70-6.67 (m, 1H), 4.47-4.45 (m, 1H), 3.92-3.86 (m, 1H), 1.26 (d, J=6.4, 3H). ¹H NMR (400 MHz, CDCl₃) δ 9.74 (s, 1H), 7.47-7.45 (m, 1H), 7.37-7.33 (m, 1H), 7.26-7.22 (m, 2H), 7.01-6.99 (m, 1H), 6.70-6.67 (m, 1H), 4.47-4.45 (m, 1H), 3.92-3.86 (m, 1H), 1.26 (d, J=6.4, 3H).

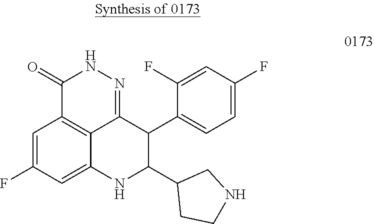 [SMILES: O=c1[nH]nc2c3c(cc(F)cc13)NC(C1CCNC1)C2c1ccc(F)cc1F]

To a solution of methyl 3-(2,4-difluorophenyl)-7-fluoro-4-oxo-2-(pyrrolidin-3-yl)-2,3-dihydro-1H-quinoline-5-carboxylate (40 mg, 0.10 mmol) in MeOH (1 mL) was added N₂H₄·H₂O (50 mg, 0.99 mmol). The reaction mixture was stirred at 25° C. for 16 hours. The mixture was concentrated and the residue was purified by Prep-HPLC (Instrument: Waters Prep-HPLC, Column: Xbridge prep c18 5 um OBD 19*150 mm, A water (0.1% formic acid), B Acetonitrile 10-20% B in 8 min, hold at 100% B at for 2 min, back to 5% B with 0.5 min, stop at 13 min, flow rate: 20 ml/min, wavelength: 214/254 nm, to afford 12-(2,4-difluorophenyl)-7-fluoro-11-(pyrrolidin-3-yl)-2,3,10-triazatricyclo[7.3.1.0\hat{ }{5,13}]trideca-1,5(13),6,8-tetraen-4-one (3 mg, 8%) as a white solid. MS (ESI): mass calcd. for C₂₀H₁₇F₃N₄O, 386.14, m/z found 387.0 [M+H]⁺. ¹H NMR (400 MHz, CD₃OD) δ 7.10 (d, J=9.2 Hz, 1H), 6.93 (s, 1H), 6.76 (s, 2H), 6.67-6.53 (m, 1H), 4.25 (d, J=3.2 Hz, 1H), 3.52 (s, 3H), 3.05 (d, J=10.4 Hz, 2H), 2.59-2.42 (m, 1H), 2.29-1.73 (m, 2H).

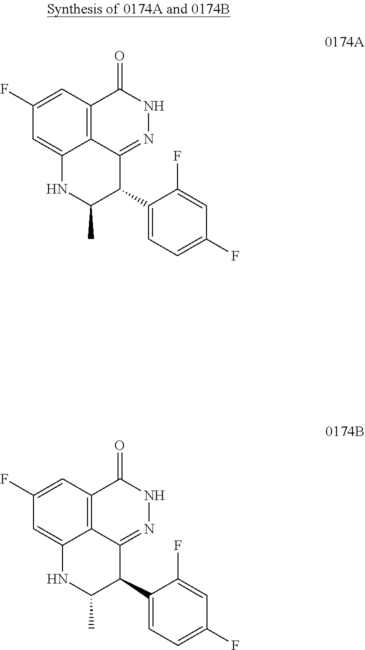 [SMILES: C[C@H]1Nc2cc(F)cc3c(=O)[nH]nc(c23)[C@@H]1c1ccc(F)cc1F ; C[C@@H]1Nc2cc(F)cc3c(=O)[nH]nc(c23)[C@H]1c1ccc(F)cc1F]

Step 1: To a solution of methyl 2-[2-(2,4-difluorophenyl)acetyl]-5-fluoro-3-nitrobenzoate (400 mg, 1.13 mmol) in THF/MeOH=5/1 (6 mL) at 0° C. was added acetaldehyde (200 mg, 4.53 mmol) and TiCl₃ in HCl (4 mL) under N₂. The reaction mixture was stirred at 40° C. for 16 h. The reaction mixture was quenched with water (20 mL) and extracted with EtOAc (30 mL×3). The combined organic layer washed with brine (30 mL), dried over Na₂SO₄, concentrated to get the residue. The residue was purified by column chromatography on silica gel (12 g) eluting with EtOAc in petroleum ether (29%), fraction with MS signal of desired product was collected and concentrated to get methyl 3-(2,4-difluorophenyl)-7-fluoro-2-methyl-4-oxo-1,2,3,4-tetrahydroquinoline-5-carboxylate (90 mg, 23%) as a yellow oil. MS (ESI): mass calcd. for C₁₈H₁₄F₃NO₃ 349.09, m/z found 350.1 [M+H]⁺.

Step 2: To the mixture of methyl 3-(2,4-difluorophenyl)-7-fluoro-2-methyl-4-oxo-2,3-dihydro-1H-quinoline-5-carboxylate (90 mg, 0.26 mmol) in MeOH (4 mL) was added N₂H₄·H₂O (4 mL). The reaction mixture was stirred at 25° C. for 3 h. The reaction mixture was concentrated to get the residue. The residue was purified by prep-HPLC (Chromatographic columns: Xbridge 5 u-C18 150×19 mm, 5 um Mobile Phase A: ACN-H₂O (0.1% formic acid), B (Acetonitrile), flow rate: 20 mL/min, wavelength: 214/254 nm, to get 9-(2,4-difluorophenyl)-5-fluoro-8-methyl-2,7,8,9-tetrahydro-3H-pyrido[4,3,2-de]phthalazin-3-one (45 mg, 53%) as a white solid. MS (ESI): mass calcd. for C₁₇H₁₂F₃N₃O 331.19, m/z found 332.1 [M+H]⁺.

Step 3: 9-(2,4-difluorophenyl)-5-fluoro-8-methyl-2,7,8,9-tetrahydro-3H-pyrido[4,3,2-de]phthalazin-3-one (45 mg, 0.14 mmol) was split via prep-SFC (Daicel CHIRALPAK AD_3 3*150 mm, 3 um mobile phase A/B: CO₂/MeOH (0.1% EDA))=75/25, flow rate: 2.0 mL/min, column temp: 37 degree) to give (8R,9R)-9-(2,4-difluorophenyl)-5-fluoro-8-methyl-2,7,8,9-tetrahydro-3H-pyrido[4,3,2-de]phthalazin-3-one (0174A, 7.9 mg, 18%) as a white solid and (8S,9S)-9-(2,4-difluorophenyl)-5-fluoro-8-methyl-2,7,8,9-tetrahydro-3H-pyrido[4,3,2-de]phthalazin-3-one (0174B, 7.8 mg, 17%) as a white solid. MS (ESI): mass calcd. for C₁₇H₁₂F₃N₃O 331.19, m/z found 332.1 [M+H]⁺.

0174A: ¹H NMR (400 MHz, CDOD) δ 7.24-7.09 (m, 2H), 7.02-6.89 (m, 2H), 6.84-6.79 (m, 1H), 4.09-4.05 (m, 1H), 3.81-3.71 (m, 1H), 1.20 (d, J=6.4 Hz, 3H).

0174B: ¹H NMR (400 MHz, CDOD) δ 7.24-7.09 (m, 2H), 7.01-6.91 (m, 2H), 6.83-6.78 (m, 1H), 4.09-4.04 (m, 1H), 3.81-3.71 (m, 1H), 1.19 (d, J=6.4 Hz, 3H).

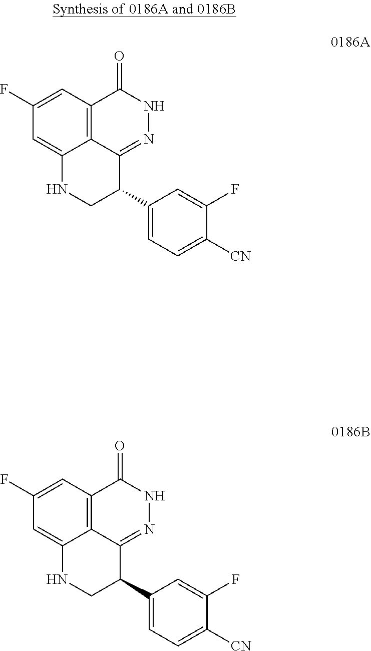 [SMILES: N#Cc1ccc([C@@H]2CNc3cc(F)cc4c(=O)[nH]nc2c34)cc1F ; N#Cc1ccc([C@H]2CNc3cc(F)cc4c(=O)[nH]nc2c34)cc1F]

Step 1: To a solution of 6-fluoro-4-nitro-3H-2-benzofuran-1-one (3.0 g, 15.2 mmol) in THE (30 mL) was added 2-fluoro-4-formylbenzonitrile (2.7 g, 18.2 mmol), TEA (9.2 g, 91.2 mmol), Ac₂O (23.3 g, 228.0 mmol). The reaction mixture was stirred at 80° C. for 3 h. While the reaction was quenched by H₂O (100 mL) at ice bath, solid precipitation was produced. The mixture was filtrated and the solid was washed by EtOH (15 mL*2) and EA (15 mL*2) to afford the product (3.0 g, 54%) as a yellow solid. MS (ESI): mass calcd. for C₁₆H₆F₂N₂O₄, 328.03, m/z found 329.0 [M+H]⁺.

Step 2: To a solution of 2-fluoro-4-{[(1Z)-5-fluoro-7-nitro-3-oxo-2-benzofuran-1-ylidene]methyl}benzonitrile (2.5 g, 7.6 mmol) in DCM (50 mL) and THE (50 mL) was added HCl/MeOH (4M, 19 mL, 76.0 mmol). The reaction mixture was stirred at 70° C. for 16 h. The reaction mixture was concentrated under reduced pressure to afford the product (2.5 g, 82%) as a yellow solid. MS (ESI): mass calcd. for C₁₇H₁₀F₂N₂O₅, 360.06, m/z found 361.0 [M+H]⁺.

Step 3: To a solution of methyl 2-[2-(4-cyano-3-fluorophenyl) acetyl]-5-fluoro-3-nitrobenzoate (950 mg, 2.64 mmol) in THE (20 mL) and MeOH (4 mL) was added HCHO (720 mg, 7.91 mmol), TiCl₃ (12.5 g, 15.82 mmol). The reaction mixture was stirred at 40° C. for 3 h. The reaction mixture was quenched with H₂O (50 mL) and extracted with EA (50 ml*2). The organic layer was washed with brine (50 mL), dried over Na₂SO₄, and filtered. The filtrate was concentrated to give a residue which was purified by silica gel column chromatography eluted with PE:EA (4:1) to afford product (300 mg, 32%) as a yellow solid. MS (ESI): mass calcd. for C₁₈H₁₂F₂N₂O₃, 342.08, m/z found 343.0 [M+H]⁺.

Step 4: To a solution of methyl 3-(4-cyano-3-fluorophenyl)-7-fluoro-4-oxo-2,3-dihydro-1H-quinoline-5-carboxylate (300 mg, 0.88 mmol) in MeOH (5 mL) was added 85% hydrazinium hydroxide solution (1.1 g, 17.53 mmol). The mixture was stirred at 20° C. for 1 h. The reaction mixture was concentrated under reduced pressure vacuum to afford a residue was purified by flash column with PE:EA (4:1) to afford product (150 mg, 52%). The product was separated by Apparatus: SFC 80, Column: Daicel CHIRALCEL IE, 250 mm, 30 mm, 10 μm, Mobile phase: CO₂/MeOH [0.2% NH₃ (7M Solution in MeOH)]=80/20, Flow rate: 2.0 g/min, wavelength: UV 214 nm, Temperature: 35° C.) to afford product 45.3 mg (0186) as a white solid and 22.2 mg (0186B) as a white solid.

0186A: MS (ESI): mass calcd. for C₁₇H₁₀F₂N₄O, 324.08, m/z found 325.0 [M+H]⁺. ¹H NMR (400 MHz, DMSO-d₆) δ 12.36 (s, 1H), 7.90-7.83 (m, 1H), 7.49 (dd, J=10.8, 1.2 Hz, 1H), 7.41 (s, 1H), 7.26 (dd, J=8.0, 1.2 Hz, 1H), 7.02 (dd, J=9.2, 2.4 Hz, 1H), 6.84 (dd, J=11.2, 2.4 Hz, 1H), 4.37 (t, J=6.4 Hz, 1H), 3.68-3.60 (m, 2H).

0186B: MS (ESI): mass calcd. for C₁₇H₁₀F₂N₄O, 324.08, m/z found 325.0 [M+H]⁺. ¹H NMR (400 MHz, DMSO-d₆) δ 12.36 (s, 1H), 7.91-7.83 (m, 1H), 7.49 (dd, J=10.8, 1.2 Hz, 1H), 7.41 (s, 1H), 7.26 (dd, J=8.0, 1.2 Hz, 1H), 7.02 (dd, J=9.2, 2.4 Hz, 1H), 6.84 (dd, J=11.2, 2.4 Hz, 1H), 4.36 (d, J=6.0 Hz, 1H), 3.67-3.59 (m, 2H).

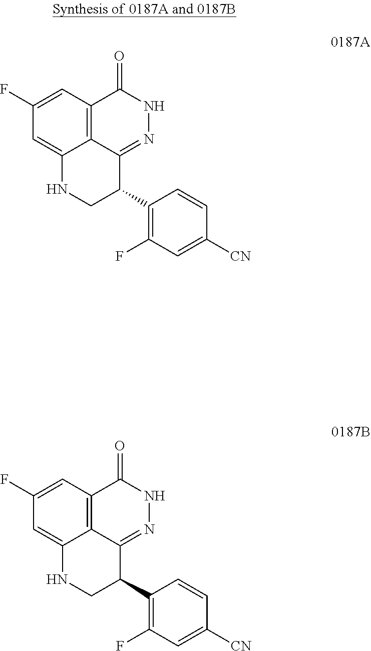 [SMILES: N#Cc1ccc([C@@H]2CNc3cc(F)cc4c(=O)[nH]nc2c34)c(F)c1 ; N#Cc1ccc([C@H]2CNc3cc(F)cc4c(=O)[nH]nc2c34)c(F)c1]

Step 1: To a solution of 6-fluoro-4-nitro-3H-2-benzofuran-1-one (5.0 g, 25.4 mmol) in THE (50 mL) was added 3-fluoro-4-formylbenzonitrile (4.6 g, 30.4 mmol), TEA (15.4 g, 152.3 mmol) and Ac₂O (38.9 g, 381.0 mmol). The reaction mixture was stirred at 80° C. for 3 h. While the reaction mixture was quenched by H₂O (200 mL), solid precipitated. Then filtrated, the solid was washed with EtOH (25 mL*2), EA (25 mL*2). The solid was dried under vacuum to afford product (6.0 g, 69%) as a yellow solid. MS (ESI): mass calcd. for C₁₆H₆F₂N₂O₄, 328.03, m/z found 329.0 [M+H]⁺.

Step 2: To a solution of 3-fluoro-4-{[(1Z)-5-fluoro-7-nitro-3-oxo-2-benzofuran-1-ylidene]methyl}benzonitrile (6.0 g, 18.3 mmol) in DCM (20 mL) and THF (50 mL) was added HCl/MeOH (4M, 91.5 mL, 366.0 mmol). The reaction mixture was stirred at 70° C. for 16 h. The reaction mixture was concentrated under reduced pressure vacuum to afford product (4.5 g, 61%) as a yellow solid.

Step 3: To a solution of methyl 2-[2-(4-cyano-2-fluorophenyl) acetyl]-5-fluoro-3-nitrobenzoate (1000 mg, 2.78 mmol) in THE (20 mL) and MeOH (4 mL) was added HCHO (250 mg, 8.33 mmol), TiCl₃ (13.2 g, 16.65 mmol). The reaction mixture was stirred at 40° C. for 3 h. The reaction mixture was quenched with H₂O (20 mL) and extracted with EA (20 mL*2). The organic layer was washed with brine (20 mL), dried over Na₂SO₄, and filtered. The filtrate was concentrated to give a residue which was purified by silica gel column chromatography eluted with PE:EA (4:1) to afford product (300 mg, 31%) as a yellow solid.

Step 4: To a solution of methyl 3-(4-cyano-2-fluorophenyl)-7-fluoro-4-oxo-2,3-dihydro-1H-quinoline-5-carboxylate (230 mg, 0.672 mmol) in MeOH (5 mL) was added 85% hydrazinium hydroxide solution (791.0 mg, 13.44 mmol). The mixture was stirred at 20° C. for 1 h. The reaction mixture was concentrated under reduced pressure vacuum to afford a residue was purified by flash column with PE:EA (4:1) to afford product (200 mg, 91%). The product was separated by Apparatus: SFC 80, Column: Daicel CHIRALCEL OD, 250 mm, 30 mm, 10 μm, Mobile phase: CO₂/MeOH [0.2% NH₃ (7M Solution in MeOH)]=80/20, Flow rate: 2.0 g/min, Wavelength: UV 214 nm, Temperature: 35° C.) to afford product 75.2 mg (0187) as a white solid and 87.3 mg (0187B) as a white solid.

0187A: MS (ESI): mass calcd. for C₁₇H₁₀F₂N₄O, 324.08, m/z found 325.0 [M+H]⁺. ¹H NMR (400 MHz, DMSO-d6) δ 12.35 (s, 1H), 7.90 (dd, J=10.4, 1.6 Hz, 1H), 7.65 (dd, J=8.0, 1.6 Hz, 1H), 7.44 (s, 1H), 7.38 (t, J=7.6 Hz, 1H), 7.03 (dd, J=9.2, 2.4 Hz, 1H), 6.86 (dd, J=11.6, 2.4 Hz, 1H), 4.51 (dd, J=8.8, 4.8 Hz, 1H), 3.71-3.53 (m, 2H).

0187B: MS (ESI): mass calcd. for C₁₇H₁₀F₂N₄O, 324.08, m/z found 325.0 [M+H]⁺. ¹H NMR (400 MHz, DMSO-d6) δ 12.35 (s, 1H), 7.89 (dd, J=10.4, 1.2 Hz, 1H), 7.65 (dd, J=8.0, 1.2 Hz, 1H), 7.44 (s, 1H), 7.38 (t, J=7.6 Hz, 1H), 7.03 (dd, J=9.2, 2.4 Hz, 1H), 6.86 (dd, J=11.2, 2.4 Hz, 1H), 4.51 (dd, J=8.8, 4.8 Hz, 1H), 3.69-3.63 (m, 1H), 3.61-3.53 (m, 1H).

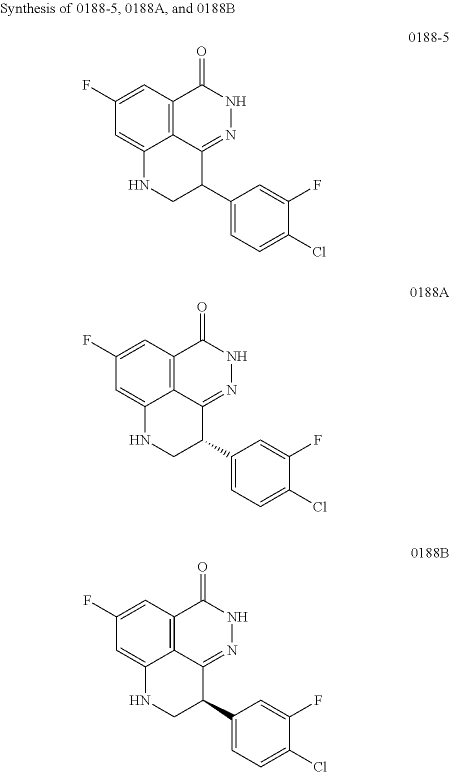 [SMILES: O=c1[nH]nc2c3c(cc(F)cc13)NCC2c1ccc(Cl)c(F)c1 ; O=c1[nH]nc2c3c(cc(F)cc13)NC[C@H]2c1ccc(Cl)c(F)c1 ; O=c1[nH]nc2c3c(cc(F)cc13)NC[C@@H]2c1ccc(Cl)c(F)c1]

Step 1: To a solution of methyl 2-[2-(4-chloro-3-fluorophenyl) acetyl]-5-fluoro-3-nitrobenzoate (2 g, 5.4 mmol) and paraformaldehyde (490 mg, 16.2 mmol) in THF (20 mL) and MeOH (4 mL) were added TiCl₃ in HCl (24 mL) under N₂. The reaction mixture was stirred at 40° C. for 3 h. The reaction mixture was quenched with water (20 mL) and extracted with EtOAc (30 mL×3). The combined organic layer washed with brine (30 mL), dried over Na₂SO₄, concentrated to get the residue. The residue was purified by column chromatography on silica gel (25 g) eluting with EtOAc in petroleum ether (25%), fraction with MS signal of desired product was collected and concentrated to get methyl 3-(4-chloro-3-fluorophenyl)-7-fluoro-4-oxo-1,2,3,4-tetrahydroquinoline-5-carboxylate (420 mg, 22%) as a yellow oil. MS (ESI): mass calcd. for C₁₇H₁₂ClF₂NO₃ 351.05, m/z found 352.0 [M+H]⁺.

Step 2: To a solution of methyl 3-(4-chloro-3-fluorophenyl)-7-fluoro-4-oxo-2,3-dihydro-1H-quinoline-5-carboxylate (420 mg, 1.19 mmol) in MeOH (15 mL) was added N₂H₄·H₂O (598 mg, 11.94 mmol) under N₂. The reaction mixture was stirred at 25° C. for 16 h. The reaction mixture was directly concentrated to get the residue. The residue was purified by prep-HPLC (Chromatographic columns: Xbridge 5 u-C18 150×19 mm, 5 um Mobile Phase A: ACN-H₂O, B (Acetonitrile), flow rate: 20 mL/min, wavelength: 214/254 nm, to get 9-(4-chloro-3-fluorophenyl)-5-fluoro-2,7,8,9-tetrahydro-3H-pyrido[4,3,2-de]phthalazin-3-one (“0188-5”; 200 mg, 50%) as a yellow solid. MS (ESI): mass calcd. for C₁₆H₁₀ClF₂N₃O 333.05, m/z found 334.0 [M+H]⁺. ¹H NMR (400 MHz, MeOD) δ 7.37 (t, J=8.0 Hz, 1H), 7.37 (td, J=8.0, 4.0 Hz, 1H), 7.03-6.98 (m, 1H), 6.80 (dd, J=10.4, 2.4 Hz, 1H), 4.30-4.15 (m, 1H), 3.72-3.60 (m, 2H).

Step 3: 9-(4-chloro-3-fluorophenyl)-5-fluoro-2,7,8,9-tetrahydro-3H-pyrido[4,3,2-de]phthalazin-3-one (“0188-5”; 300 mg, 0.72 mmol) was split via prep-SFC (Daicel CHIRALPAK AD_3 3*150 mm, 3 um mobile phase A/B: CO₂/MeOH (0.1% EDA))=70/30, flow rate: 2.0 mL/min, column temp: 37 degree) to give (R)-9-(4-chloro-3-fluorophenyl)-5-fluoro-2,7,8,9-tetrahydro-3H-pyrido[4,3,2-de]phthalazin-3-one (0188A, 87.8 mg, 29%) as an white solid and (S)-9-(4-chloro-3-fluorophenyl)-5-fluoro-2,7,8,9-tetrahydro-3H-pyrido[4,3,2-de]phthalazin-3-one (0188B, 8778 mg, 26%) as a white solid.

0188A: MS (ESI): mass calcd. for C₁₆H₁₀ClF₂N₃O 333.05, m/z found 334.0 [M+H]⁺. ¹H NMR (400 MHz, MeOD) δ 7.37 (t, J=8.0 Hz, 1H), 7.18-7.09 (m, 2H), 7.03-6.97 (m, 1H), 6.83-6.74 (m, 1H), 4.28-4.18 (m, 1H), 3.73-3.58 (m, 2H).

0188B: MS (ESI): mass calcd. for C₁₆H₁₀ClF₂N₃O 333.05, m/z found 334.0 [M+H]⁺. ¹H NMR (400 MHz, MeOD) δ 7.37 (t, J=8.0 Hz, 1H), 7.20-7.06 (m, 2H), 7.03-6.97 (m, 1H), 6.92-6.78 (m, 1H), 4.30-4.16 (m, 1H), 3.72-3.59 (m, 2H).

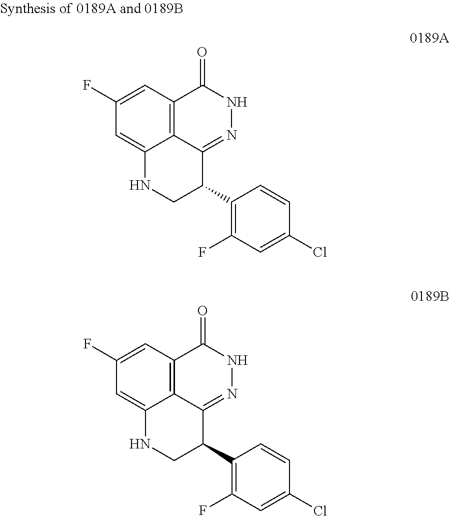 [SMILES: O=c1[nH]nc2c3c(cc(F)cc13)NC[C@H]2c1ccc(Cl)cc1F ; O=c1[nH]nc2c3c(cc(F)cc13)NC[C@@H]2c1ccc(Cl)cc1F]

Step 1: To a solution of 6-fluoro-4-nitro-3H-2-benzofuran-1-one (10.0 g, 50.7 mmol) in THE (100 mL) was added 4-chloro-2-fluorobenzaldehyde (9.7 g, 60.8 mmol), TEA (30.8 g, 30.4 mmol), Ac₂O (62.1 g, 60.8 mmol). The reaction mixture was stirred at 80° C. for 3 h. While the reaction mixture was quenched by H₂O (500 mL). After filtration, the solid was collected and washed with EtOH (2*40 mL), EA (2*40 mL). The solid was dried under vacuum to afford product (9.0 g) as a yellow solid. MS (ESI): mass calcd. for C₁₅H₆ClF₂NO₄, 337.00, m/z found 337.9 [M+H]⁺.

Step 2: To a solution of (3Z)-3-[(4-chloro-2-fluorophenyl) methylidene]-6-fluoro-4-nitro-2-benzofuran-1-one (9.0 g, 26.7 mmol) in THE (150 mL) was added HCl/MeOH (67 mL, 268.0 mmol). The reaction mixture was stirred at 70° C. for 16 h. The reaction mixture was concentrated, then diluted with EA (150 mL), H2O (200 mL), extracted with EA (2*150 mL). The organic layer was washed with brine (150 mL), dried over anhydrous Na₂SO₄, concentrated under vacuum. The residue was purified by silica gel column chromatography eluted with PE/EA (8:1) to afford product (6.0 g) as a yellow solid. MS (ESI): mass calcd. for C₁₆H₁₀ClF₂NO₅, 369.02, m/z found 392.0 [M+Na]⁺.

Step 3: To a solution of methyl 2-[2-(4-chloro-2-fluorophenyl) acetyl]-5-fluoro-3-nitrobenzoate (370 mg, 1.00 mmol) in THE (10 mL) and MeOH (2 mL) was added formaldehyde aqueous solution (300 mg, 3.00 mmol), TiCl₃ (4.7 g, 6.0 mmol). The reaction mixture was stirred at 40° C. for 3 h. The reaction mixture was quenched by H₂O (50 mL), extracted with EA (2*50 mL). The organic layer was washed with brine (50 mL), dried over anhydrous Na₂SO₄, concentrated. The residue was purified by silica gel column chromatography eluted with PE/EA (5:1) to afford product (180 mg) as a yellow solid. MS (ESI): mass calcd. for C₁₇H₁₂ClF₂NO₃, 351.05, m/z found 352.0 [M+H]⁺.

Step 4: To a solution of methyl 3-(4-chloro-2-fluorophenyl)-7-fluoro-4-oxo-2,3-dihydro-1H-quinoline-5-carboxylate (180 mg, 0.51 mmol) in MeOH (5 mL) was added NH₂NH₂·H₂O (647 mg, 10.24 mmol). The reaction mixture was stirred at 25° C. for 3 h. The reaction mixture was quenched by H₂O (20 mL), extracted with EA (2*20 mL). The organic layer was washed with brine (30 mL), dried over anhydrous Na₂SO₄, concentrated under vacuum. The residue was purified by silica gel column chromatography eluted with PE/EA (1:1) to afford crude product (90 mg). The crude product was separated by Apparatus: SFC 150, Column: Daicel CHIRALCEL OJ, 250 mm, 30 mm I.D., 10 μm, Mobile phase: CO₂/MeOH [0.2% NH₃ (7M Solution in MeOH)]=80/20, Flow rate: 2.0 g/min, Wave length: UV 214 nm, Temperature: 35° C.) this resulted product 17.6 mg (0189) as a white solid, 25.0 mg (0189B) as a white solid.

0189A: MS (ESI): mass calcd. for C₁₆H₁₀ClF₂N₃O, 333.05, m/z found 334.0 [M+H]⁺. ¹H NMR (400 MHz, MeOD) δ 7.32-7.06 (m, 3H), 7.02-6.92 (1H), 6.82 (dd, J=10.8, 2.4 Hz, 1H), 4.54-4.44 (m, 1H), 3.72-3.54 (m, 2H).

0189B: MS (ESI): mass calcd. for C₁₆H₁₀ClF₂N₃O, 333.05, m/z found 334.0 [M+H]⁺. ¹H NMR (400 MHz, MeOD) δ 7.27-7.07 (m, 3H), 7.03-6.95 (m, 1H), 6.82 (dd, J=10.8, 2.4 Hz, 1H), 4.54-4.43 (m, 1H), 3.71-3.54 (m, 2H).

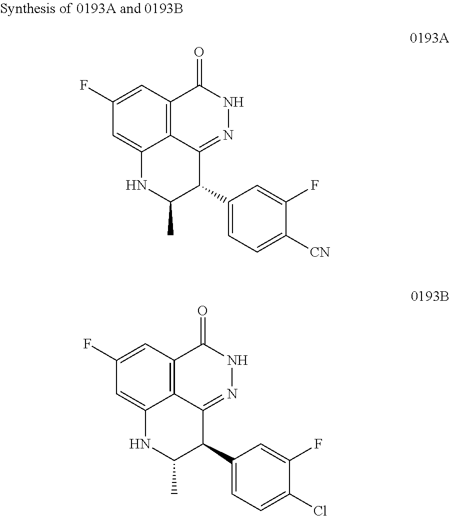 [SMILES: C[C@H]1Nc2cc(F)cc3c(=O)[nH]nc(c23)[C@@H]1c1ccc(C#N)c(F)c1 ; C[C@@H]1Nc2cc(F)cc3c(=O)[nH]nc(c23)[C@H]1c1ccc(Cl)c(F)c1]

Step 1: To a solution of 6-fluoro-4-nitro-3H-2-benzofuran-1-one (3.0 g, 15.2 mmol) in THE (30 mL) were added 2-fluoro-4-formylbenzonitrile (2.7 g, 18.2 mmol), TEA (9.2 g, 91.2 mmol) and Ac₂O (23.3 g, 228.0 mmol). The reaction mixture was stirred at 80° C. for 3 h. While the reaction was quenched by H₂O (100 mL) at ice bath, solid precipitation was produced. The mixture was filtrated and the solid was washed by EtOH (15 mL*2) and EA (15 mL*2) to afford the product (3.0 g, 54%) as a yellow solid. MS (ESI): mass calcd. for C₁₆H₆F₂N₂O₄, 328.03, m/z found 329.0 [M+H]⁺.

Step 2: To a solution of 2-fluoro-4-{[(1Z)-5-fluoro-7-nitro-3-oxo-2-benzofuran-1-ylidene]methyl}benzonitrile (2.5 g, 7.6 mmol) in DCM (50 mL) and THE (50 mL) was added HCl/MeOH (4M, 19 mL, 76.0 mmol). The reaction mixture was stirred at 70° C. for 16 h. The reaction mixture was concentrated under reduced pressure vacuum to afford the product (2.5 g, 82%) as a yellow solid. MS (ESI): mass calcd. for C₁₇H₁₀F₂N₂O₅, 360.06, m/z found 361.0 [M+H]⁺.

Step 3: To a solution of methyl 2-[2-(4-cyano-3-fluorophenyl) acetyl]-5-fluoro-3-nitrobenzoate (800 mg, 2.22 mmol) in THE (20 mL) and MeOH (4 mL) were added CH₃CHO (294 mg, 6.66 mmol) and TiCl₃ (10.6 g, 13.32 mmol). The reaction mixture was stirred at 40° C. for 3 h. The reaction mixture was quenched with H₂O (50 mL) and extracted with EA (50 ml*2). The organic layer was washed with brine (50 mL), dried over Na₂SO₄, and filtered. The filtrate was concentrated to give a residue which was purified by silica gel column chromatography eluted with PE:EA (4:1) to afford product (320 mg, 38%) as a yellow solid. MS (ESI): mass calcd. for C₁₉H₁₄F₂N₂O₃, 356.10, m/z found 357.0 [M+H]⁺.

Step 4: To a solution of methyl 3-(4-cyano-3-fluorophenyl)-7-fluoro-2-methyl-4-oxo-2,3-dihydro-1H-quinoline-5-carboxylate (320 mg, 0.90 mmol) in MeOH (10 mL) was added 85% hydrazinium hydroxide solution (1.1 g, 17.96 mmol). The mixture was stirred at 20° C. for 1 h. The reaction mixture was concentrated under reduced pressure vacuum to afford a residue which was purified by flash column with PE:EA (4:1) to afford product (150 mg, 58%). Then was separated by Apparatus: SFC 80, Column: Daicel CHIRALCEL IE, 250 mm, 30 mm, 10 μm, Mobile phase: CO₂/MeOH [0.2% NH₃ (7M Solution in MeOH)]=80/20, Flow rate: 2.0 g/min, wavelength: UV 214 nm, Temperature: 35° C.) to afford product 52.5 mg (0193) as a white solid and 56.6 mg (0193B) as a white solid.

0193A: MS (ESI): mass calcd. for C₁₈H₁₂F₂N₄O, 338.10, m/z found 339.0 [M+H]⁺. ¹H NMR (400 MHz, DMSO-d6) δ 12.29 (s, 1H), 7.90 (t, J=7.2 Hz, 1H), 7.54 (d, J=10.8 Hz, 1H), 7.42 (s, 1H), 7.34 (d, J=8.0 Hz, 1H), 7.00 (dd, J=9.2, 2.4 Hz, 1H), 6.83 (dd, J=11.2, 2.4 Hz, 1H), 4.03 (d, J=10.0 Hz, 1H), 3.86-3.77 (m, 1H), 1.07 (d, J=6.0 Hz, 3H).

0193B: MS (ESI): mass calcd. for C₁₈H₁₂F₂N₄O, 338.10, m/z found 339.0 [M+H]⁺. ¹H NMR (400 MHz, DMSO-d6) δ 12.29 (s, 1H), 7.90 (t, J=7.6 Hz, 1H), 7.54 (d, J=10.4 Hz, 1H), 7.42 (s, 1H), 7.34 (d, J=8.4 Hz, 1H), 7.00 (dd, J=9.2, 2.4 Hz, 1H), 6.83 (dd, J=11.2, 2.4 Hz, 1H), 4.03 (d, J=9.6 Hz, 1H), 3.86-3.77 (m, 1H), 1.07 (d, J=6.0 Hz, 3H).

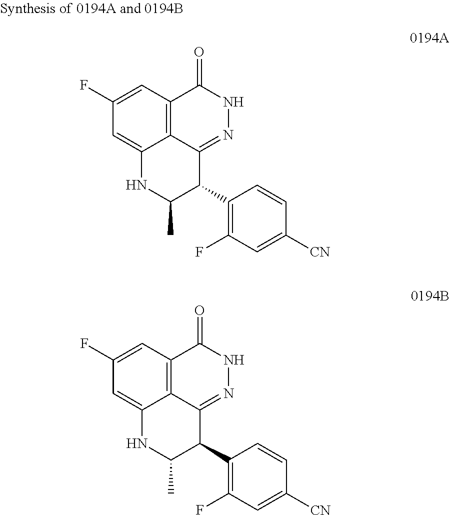 [SMILES: C[C@H]1Nc2cc(F)cc3c(=O)[nH]nc(c23)[C@@H]1c1ccc(C#N)cc1F ; C[C@@H]1Nc2cc(F)cc3c(=O)[nH]nc(c23)[C@H]1c1ccc(C#N)cc1F]

Step 1: To a solution of 6-fluoro-4-nitro-3H-2-benzofuran-1-one (5.0 g, 25.4 mmol) in THE (50 mL) was added 3-fluoro-4-formylbenzonitrile (4.6 g, 30.4 mmol), TEA (15.4 g, 152.3 mmol) and Ac₂O (38.9 g, 381.0 mmol). The reaction mixture was stirred at 80° C. for 3 h. While the reaction was quenched by H₂O (200 mL) at ice bath, solid precipitation was produced. The mixture was filtrated and the solid was washed by EtOH (15 mL*2) and EA (15 mL*2) to afford the product (6.0 g, 69%) as a yellow solid. MS (ESI): mass calcd. for C₁₆H₆F₂N₂O₄, 328.03, m/z found 329.0 [M+H]⁺.

Step 2: To a solution of 3-fluoro-4-{[(1Z)-5-fluoro-7-nitro-3-oxo-2-benzofuran-1-ylidene]methyl}benzonitrile (6.0 g, 18.3 mmol) in DCM (20 mL) and THF (50 mL) was added HCl/MeOH (91.5 mL, 366.0 mmol). The reaction mixture was stirred at 70° C. for 16 h. The reaction mixture was concentrated under reduced pressure vacuum to afford product (4.5 g, 61%) as a yellow solid.

Step 3: To a solution of methyl 2-[2-(4-cyano-2-fluorophenyl) acetyl]-5-fluoro-3-nitrobenzoate (1000 mg, 2.78 mmol) in THE (20 mL) and MeOH (4 mL) was added CH₃CHO (367 mg, 8.33 mmol), TiCl₃ (13.2 g, 16.65 mmol). The reaction mixture was stirred at 40° C. for 3 h. The reaction mixture was quenched with H₂O (20 mL) and extracted with EA (20 mL*2). The organic layer was washed with brine (20 mL), dried over Na₂SO₄, and filtered. The filtrate was concentrated to give a residue which was purified by silica gel column chromatography eluted with PE:EA (5:1) to afford product (600 mg, 60%) as a yellow solid.

Step 4: To a solution of methyl 3-(4-cyano-2-fluorophenyl)-7-fluoro-2-methyl-4-oxo-2,3-dihydro-1H-quinoline-5-carboxylate (400 mg, 1.23 mmol) in MeOH (5 mL) was added 85% hydrazinium hydroxide solution (199 mg, 3.37 mmol). The mixture was stirred at 20° C. for 1 h. The reaction mixture was concentrated under reduced pressure vacuum to afford a residue was purified by flash column with PE:EA (4:1) to afford product (200 mg, 91%). The product was separated by Apparatus: SFC 80, Column: Daicel CHIRALCEL OD, 250 mm, 30 mm, 10 μm, Mobile phase: CO₂/MeOH [0.2% NH₃ (7M Solution in MeOH)]=80/20, Flow rate: 2.0 g/min, Wavelength: UV 214 nm, Temperature: 35° C.) to afford product 42.0 mg (0194A) as a white solid and 38.9 mg (0194B) as a white solid.

0194A: MS (ESI): mass calcd. for C₁₈H₁₂F₂N₄O, 338.10, m/z found 339.0 [M+H]⁺. ¹H NMR (400 MHz, DMSO-d6) δ 12.29 (s, 1H), 7.89 (dd, J=10.0, 1.2 Hz, 1H), 7.71 (dd, J=8.0, 1.2 Hz, 1H), 7.57 (t, J=7.6 Hz, 1H), 7.45 (s, 1H), 7.02 (dd, J=9.2, 2.4 Hz, 1H), 6.84 (dd, J=11.2, 2.4 Hz, 1H), 4.17 (d, J=10.8 Hz, 1H), 3.87-3.77 (m, 1H), 1.10 (d, J=6.0 Hz, 3H).

0194B: MS (ESI): mass calcd. for C₁₈H₁₂F₂N₄O, 338.10, m/z found 339.0 [M+H]⁺. ¹H NMR (400 MHz, DMSO-d6) δ 12.29 (s, 1H), 7.89 (dd, J=10.4, 1.2 Hz, 1H), 7.71 (dd, J=8.0, 1.2 Hz, 1H), 7.57 (t, J=7.6 Hz, 1H), 7.45 (s, 1H), 7.02 (dd, J=9.2, 2.4 Hz, 1H), 6.84 (dd, J=11.2, 2.4 Hz, 1H), 4.17 (d, J=10.8 Hz, 1H), 3.86-3.78 (m, 1H), 1.10 (d, J=6.4 Hz, 3H).

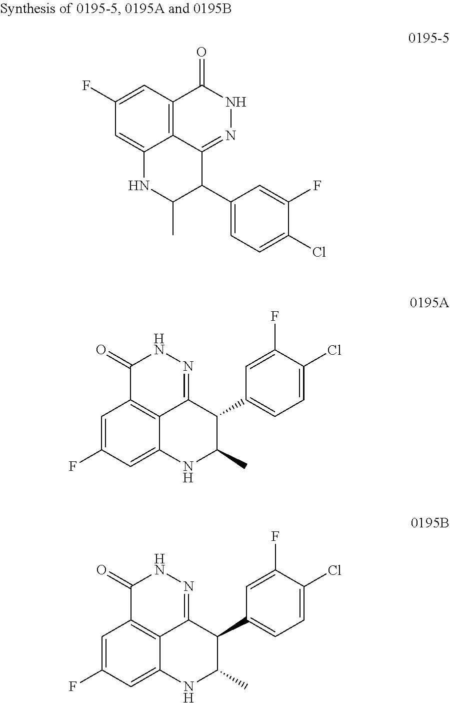 [SMILES: CC1Nc2cc(F)cc3c(=O)[nH]nc(c23)C1c1ccc(Cl)c(F)c1 ; C[C@H]1Nc2cc(F)cc3c(=O)[nH]nc(c23)[C@@H]1c1ccc(Cl)c(F)c1 ; C[C@@H]1Nc2cc(F)cc3c(=O)[nH]nc(c23)[C@H]1c1ccc(Cl)c(F)c1]

Step 1: To a solution of methyl 2-(2-(4-chloro-3-fluorophenyl)acetyl)-5-fluoro-3-nitrobenzoate (1 g, 2.7 mmol) and acetaldehyde (0.59 g, 13.5 mmol) in THE (12 mL) and MeOH (2 mL) was added TiCl₃ in HCl (14 mL) under N₂. The reaction mixture was stirred at 40° C. for 3 h. The reaction mixture was quenched with water (20 mL) and extracted with EtOAc (30 mL×3). The combined organic layer was washed with brine (30 mL), dried over Na₂SO₄, concentrated to get the residue. The residue was purified by column chromatography on silica gel (25 g) eluting with EtOAc in petroleum ether (25%), fraction with MS signal of desired product was collected and concentrated to get methyl 3-(4-chloro-3-fluorophenyl)-7-fluoro-2-methyl-4-oxo-1,2,3,4-tetrahydroquinoline-5-carboxylate (300 mg, 30%) as a white solid. MS (ESI): mass calcd. for C₁₈H₁₄ClF₂NO₃ 365.06, m/z found 366.1 [M+H]⁺.

Step 2: To a solution of methyl 3-(4-chloro-3-fluorophenyl)-7-fluoro-2-methyl-4-oxo-2,3-dihydro-1H-quinoline-5-carboxylate (300 mg, 0.82 mmol) in MeOH (15 mL) was added N₂H₄·H₂O (411 mg, 8.2 mmol) under N₂. The reaction mixture was stirred at 25° C. for 3 h. The reaction mixture was concentrated to get the residue. The residue was purified by prep-HPLC (Chromatographic columns: Xbridge 5 u-C18 150×19 mm, 5 um Mobile Phase A: ACN-H₂O, B (Acetonitrile), flow rate: 20 mL/min, wavelength: 214/254 nm, to get 9-(2-chlorophenyl)-5-fluoro-2,7,8,9-tetrahydro-3H-pyrido[4,3,2-de]phthalazin-3-one (“0195-5”; 200 mg, 72%) as a white solid. MS (ESI): mass calcd. for C₁₇H₁₂ClF₂N₃O 347.06, m/z found 348.1 [M+H]⁺. ¹H NMR (400 MHz, MeOD) δ 7.43 (t, J=8.0 Hz, 1H), 7.19-7.10 (m, 2H), 7.08-7.02 (m, 1H), 6.82 (dd, J=11.2, 2.4 Hz, 1H), 3.89-3.85 (m, 1H), 3.78-3.72 (m, 1H), 1.21 (d, J=6.4 Hz, 3H).

Step 3: 9-(4-chloro-3-fluorophenyl)-5-fluoro-8-methyl-2,7,8,9-tetrahydro-3H-pyrido[4,3,2-de]phthalazin-3-one (“0195-5”; 250 mg, 0.72 mmol) was split via prep-SFC (Daicel CHIRALPAK AD_3 3*150 mm, 3 um mobile phase A/B: CO₂/MeOH (0.1% EDA))=70/30, flow rate: 2.0 mL/min, column temp: 37 degree) to give (8R,9R)-9-(4-chloro-3-fluorophenyl)-5-fluoro-8-methyl-2,7,8,9-tetrahydro-3H-pyrido[4,3,2-de]phthalazin-3-one (0195, 100.7 mg, 40%) as an off-white solid and (8S,9S)-9-(4-chloro-3-fluorophenyl)-5-fluoro-8-methyl-2,7,8,9-tetrahydro-3H-pyrido[4,3,2-de]phthalazin-3-one (0195B, 85.7 mg, 34%) as an off-white solid.

0195A: MS (ESI): mass calcd. for C₁₇H₁₂ClF₂N₃O 347.06, m/z found 348.1 [M+H]⁺. ¹H NMR (400 MHz, MeOD) δ 7.41 (t, J=8.0 Hz, 1H), 7.16-7.07 (m, 2H), 7.03 (dd, J=8.4, 1.6 Hz, 1H), 6.79 (dd, J=11.2, 2.4 Hz, 1H), 3.87-3.83 (m, 1H), 3.79-3.70 (m, 1H), 1.19 (d, J=6.4 Hz, 3H).

0195B: MS (ESI): mass calcd. for C₁₇H₁₂ClF₂N₃O 347.06, m/z found 348.1 [M+H]⁺. ¹H NMR (400 MHz, MeOD) δ 7.41 (t, J=8.0 Hz, 1H), 7.21-7.07 (m, 2H), 7.03 (dd, J=8.4, 1.6 Hz, 1H), 6.79 (dd, J=11.2, 2.4 Hz, 1H), 3.88-3.82 (m, 1H), 3.80-3.70 (m, 1H), 1.19 (d, J=6.4 Hz, 3H).

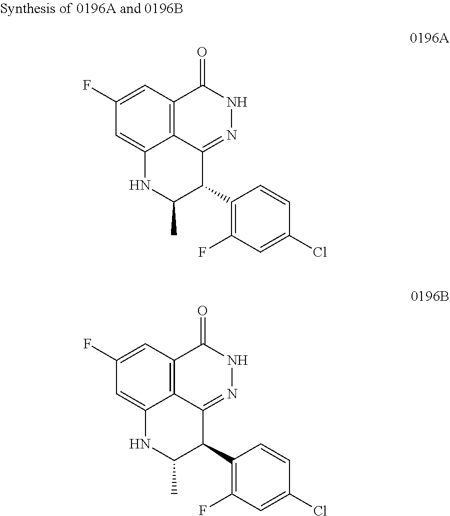 [SMILES: C[C@H]1Nc2cc(F)cc3c(=O)[nH]nc(c23)[C@@H]1c1ccc(Cl)cc1F ; C[C@@H]1Nc2cc(F)cc3c(=O)[nH]nc(c23)[C@H]1c1ccc(Cl)cc1F]

Step 1: To a solution of 6-fluoro-4-nitro-3H-2-benzofuran-1-one (10.0 g, 50.7 mmol) in THE (100 mL) was added 4-chloro-2-fluorobenzaldehyde (9.7 g, 60.8 mmol), TEA (30.8 g, 30.4 mmol), Ac₂O (62.1 g, 60.8 mmol). The reaction mixture was stirred at 80° C. for 3 h. While the reaction mixture was quenched by H₂O (500 mL). The solid was collected by filtration and washed with EtOH (2*40 mL), EA (2*40 mL). The solid was dried under vacuum to afford product (9.0 g) as a yellow solid. MS (ESI): mass calcd. for C₁₅H₆ClF₂NO₄, 337.00, m/z found 337.9 [M+H]⁺.

Step 2: To a solution of (3Z)-3-[(4-chloro-2-fluorophenyl) methylidene]-6-fluoro-4-nitro-2-benzofuran-1-one (9.0 g, 26.7 mmol) in THE (150 mL) was added HCl/MeOH (67 mL, 268.0 mmol). The reaction mixture was stirred at 70° C. for 16 h. The reaction mixture was concentrated, then diluted with EA (150 mL), H₂O (200 mL), extracted with EA (2*150 mL). The organic layer was washed with brine (150 mL), dried over anhydrous Na₂SO₄, concentrated under vacuum. The residue was purified by silica gel column chromatography eluted with PE/EA (8:1) to afford product (6.0 g) as a yellow solid. MS (ESI): mass calcd. for C₁₆H₁₀ClF₂NO₅, 369.02, m/z found 392.0 [M+Na]⁺.

Step 3: To a solution of methyl 2-[2-(4-chloro-2-fluorophenyl) acetyl]-5-fluoro-3-nitrobenzoate (500 mg, 1.35 mmol) in THE (10 mL) and MeOH (2 mL) was added acetaldehyde (72 mg, 1.62 mmol), TiCl₃ (6.4 g, 8.11 mmol). The reaction mixture was stirred at 40° C. for 3 h. The reaction mixture was quenched by H₂O (50 mL), extracted with EA (2*50 mL). The organic layer was washed with brine (50 mL), dried over anhydrous Na₂SO₄, concentrated. The residue was purified by silica gel column chromatography eluted with PE/EA (5:1) to afford product (300 mg) as a yellow solid. MS (ESI): mass calcd. for C₁₈H₁₄ClF₂NO₃, 365.06, m/z found 366.0 [M+H]⁺.

Step 4: To a solution of methyl 3-(4-chloro-2-fluorophenyl)-7-fluoro-4-oxo-2,3-dihydro-1H-quinoline-5-carboxylate (300 mg, 0.85 mmol) in MeOH (5 mL) was added NH₂NH₂·H₂O (1.1 g, 17.06 mmol). The reaction mixture was stirred at 25° C. for 3 h. The reaction mixture was quenched by H₂O (20 mL), extracted with EA (2*30 mL). The organic layer was washed with brine (30 mL), dried over anhydrous Na₂SO₄, concentrated under vacuum. The residue was purified by silica gel column chromatography eluted with PE/EA (1:1) to afford a crude product (150 mg). The crude product was separated by Apparatus: SFC 150, Column: Daicel CHIRALCEL OJ, 250 mm 30 mm I.D., 10 μm, Mobile phase: CO₂/MeOH [0.2% NH₃ (7M Solution in MeOH)]=70/30, Flow rate: 2.0 g/min, Wave length: UV 214 nm, Temperature: 35° C.) this resulted product 45.9 mg (0196) as a white solid, 49.5 mg (0196B) as a white solid.

0196A: MS (ESI): mass calcd. for C₁₇H₁₂ClF₂N₃O, 347.06, m/z found 348.0 [M+H]⁺. ¹H NMR (400 MHz, MeOD) δ 7.32-7.10 (m, 4H), 6.83 (dd, J=10.8, 2.4 Hz, 1H), 4.10 (d, J=9.6 Hz, 1H), 3.85-3.73 (m, 1H), 1.22 (d, J=6.4 Hz, 3H).

0196B: MS (ESI): mass calcd. for C₁₇H₁₂ClF₂N₃O, 347.06, m/z found 348.0 [M+H]⁺. ¹H NMR (400 MHz, MeOD) δ 7.30-7.02 (m, 4H), 6.81 (dd, J=10.8, 2.4 Hz, 1H), 4.08 (d, J=9.6 Hz, 1H), 3.86-3.71 (m, 1H), 1.20 (d, J=6.4 Hz, 3H).

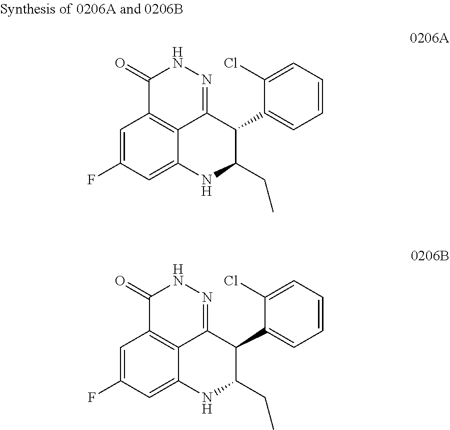 [SMILES: CC[C@H]1Nc2cc(F)cc3c(=O)[nH]nc(c23)[C@@H]1c1ccccc1Cl ; CC[C@@H]1Nc2cc(F)cc3c(=O)[nH]nc(c23)[C@H]1c1ccccc1Cl]

Step 1: A mixture of 6-fluoro-4-nitro-3H-2-benzofuran-1-one (10.0 g, 50.73 mmol) and 2-chlorobenzaldehyde (14.3 g, 101.46 mmol) in THF (500 mL) was added TEA (15.4 g, 152.19 mmol) and Ac₂O (31.1 g, 304.38 mmol). The mixture was stirred at 80° C. for 16 hours. The reaction mixture was filtered. The filter cake was washed with water (200 mL×2) and dried under reduced pressure to afford (3Z)-3-[(2-chlorophenyl)methylidene]-6-fluoro-4-nitro-2-benzofuran-1-one (10.0 g, 56%) as a yellow solid. MS (ESI): mass calcd. for C₁₅H₇ClFNO₄, 319.0, m/z found 319.9 [M+H]⁺.

Step 2: A mixture of methylidene]-6-fluoro-4-nitro-2-benzofuran-1-one (10.0 g, 31.28 mmol) in HCl/EA (500 mL, 4 m in EA) was stirred at 75° C. for 16 hours. The reaction mixture was filtered. The filter cake was washed with MeOH (100 mL×2) and dried under reduced pressure to afford methyl 2-[2-(2-chlorophenyl)acetyl]-5-fluoro-3-nitrobenzoate (10.0 g, 82%) as a yellow solid. MS (ESI): mass calcd. for C₁₆H₁₁ClFNO₅, 351.0, m/z found 352.0 [M+H]⁺.

Step 3: To a solution of methyl 2-[2-(2-chlorophenyl)acetyl]-5-fluoro-3-nitrobenzoate (2.0 g, 5.69 mmol) in MeOH (10 mL) and THE (50 mL) was added TiCl₃ (20.3 g, 34.12 mmol) and propanal (991 mg, 17.06 mmol). The reaction mixture was stirred at 40° C. for 6 hours. The mixture was diluted with water (50 mL) and extracted with EA (100 mL×3). The organic layer was concentrated and the residue was purified by flash chromatograph on silica gel column (PE:EA=3:1) to afford methyl 3-(2-chlorophenyl)-2-ethyl-7-fluoro-4-oxo-2,3-dihydro-1H-quinoline-5-carboxylate (1.0 g, 44%) as yellow oil. MS (ESI): mass calcd. for C₁₉H₁₇ClFNO₃, 361.1, m/z found 362.1 [M+H]⁺.

Step 4: To a mixture of methyl 3-(2-chlorophenyl)-2-ethyl-7-fluoro-4-oxo-2,3-dihydro-1H-quinoline-5-carboxylate (750 mg, 2.07 mmol) in MeOH (20 mL) was added N₂H₄·H₂O (199 mg, 6.22 mmol). The reaction mixture was stirred at 25° C. for 16 hours. The mixture was concentrated under reduced pressure to give a residue which was purified by reversed phase column (40% A in B; A: CH₃CN, B: 0.1% FA in water) to afford 12-(2-chlorophenyl)-11-ethyl-7-fluoro-2,3,10-triazatricyclo[7.3.1.0\hat{ }{5,13}]trideca-1,5(13),6,8-tetraen-4-one (300 mg, 40%) as yellow solid. MS (ESI): mass calcd. for C₁₈H₁₅ClFN₃O, 343.1, m/z found 344.1 [M+H]⁺.

Step 5: 12-(2-chlorophenyl)-11-ethyl-7-fluoro-2,3,10-triazatricyclo[7.3.1.0\hat{ }{5,13}]trideca-1,5(13),6,8-tetraen-4-one (300 mg, 0.87 mmol) was separated by Prep-SFC (Apparatus: SFC 150 Column: Daicel CHIRALCEL IE, 250 mm×30 mm I.D. Mobile phase: CO₂/MeOH [0.2% NH₃ (7 M Solution in MeOH)]=65/35 Flow rate: 80 g/min Wave length: UV 214 nm Temperature: 35° C.) to give (11S,12S)-12-(2-chlorophenyl)-11-ethyl-7-fluoro-2,3,10-triazatricyclo[7.3.1.0\hat{ }{5,13}]trideca-1,5(13),6,8-tetraen-4-one (57.4 mg, 19%) as white solid and (11R,12R)-12-(2-chlorophenyl)-11-ethyl-7-fluoro-2,3,10-triazatricyclo[7.3.1.0\hat{ }{5,13}]trideca-1,5(13),6,8-tetraen-4-one (32.4 mg, 11%) as a white solid.

0206A: MS (ESI): mass calcd. for C₁₈H₁₅ClFN₃O, 343.1, m/z found 344.1 [M+H]⁺. ¹H NMR (400 MHz, DMSO) δ 12.34 (s, 1H), 7.52-7.48 (m, 1H), 7.41-7.20 (m, 3H), 7.10-6.85 (m, 3H), 4.38 (d, J=7.2 Hz, 1H), 3.71-3.60 (m, 1H), 1.63-1.35 (m, 2H), 0.95 (t, J=7.2 Hz, 3H).

0206B: MS (ESI): mass calcd. for C₁₈H₁₅ClFN₃O, 343.1, m/z found 344.1 [M+H]⁺. ¹H NMR (400 MHz, DMSO) δ 12.34 (s, 1H), 7.53-7.47 (m, 1H), 7.35-7.21 (m, 3H), 7.04-6.86 (m, 3H), 4.38 (d, J=7.2 Hz, 1H), 3.70-3.61 (m, 1H), 1.64-1.35 (m, 2H), 0.95 (t, J=7.2 Hz, 3H).

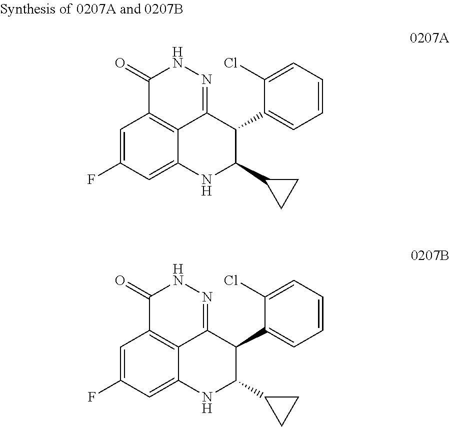 [SMILES: O=c1[nH]nc2c3c(cc(F)cc13)N[C@H](C1CC1)[C@H]2c1ccccc1Cl ; O=c1[nH]nc2c3c(cc(F)cc13)N[C@@H](C1CC1)[C@@H]2c1ccccc1Cl]

Step 1: To a solution of methyl 2-[2-(2-chlorophenyl)acetyl]-5-fluoro-3-nitrobenzoate (2.0 g, 5.69 mmol) in MeOH (10 mL) and THE (50 mL) was added TiCl₃ (20.3 g, 34.12 mmol) and cyclopropanecarbaldehyde (797 mg, 11.37 mmol). The reaction mixture was stirred at 40° C. for 6 hours. The mixture was diluted with water (50 mL) and extracted with EA (100 mL×3). The organic layer was concentrated. The residue was purified by flash chromatograph on silica gel column (PE:EA=3:1) to afford methyl 3-(2-chlorophenyl)-2-cyclopropyl-7-fluoro-4-oxo-2,3-dihydro-1H-quinoline-5-carboxylate (1.0 g, 42%) as yellow oil. MS (ESI): mass calcd. for C₂₀H₁₇ClFNO₃, 373.1, m/z found 374.0 [M+H]⁺.

Step 2: To a mixture of methyl 3-(2-chlorophenyl)-2-cyclopropyl-7-fluoro-4-oxo-2,3-dihydro-1H-quinoline-5-carboxylate (750 mg, 2.01 mmol) in MeOH (20 mL) was added N₂H₄·H₂O (193 mg, 6.02 mmol). The reaction mixture was stirred at 25° C. for 16 hours. The mixture was concentrated under reduced pressure to give a residue which was purified by reversed phase column (40% A in B; A: CH₃CN, B: 0.1% FA in water) to afford 12-(2-chlorophenyl)-11-cyclopropyl-7-fluoro-2,3,10-triazatricyclo[7.3.1.0\hat{ }{5,13}]trideca-1,5(13),6,8-tetraen-4-one (300 mg, 29%) as yellow solid. MS (ESI): mass calcd. for C₂₀H₁₇ClFNO₃, 373.1, m/z found 374.0 [M+H]⁺.

Step 3: 12-(2-chlorophenyl)-11-cyclopropyl-7-fluoro-2,3,10-triazatricyclo[7.3.1.0\hat{ }{5,13}]trideca-1,5(13),6,8-tetraen-4-one (300 mg, 0.84 mmol) was separated by Prep-SFC (Apparatus: SFC 150 Column: Daicel CHIRALCEL IE, 250 mm×30 mm I.D. Mobile phase: CO₂/MeOH [0.2% NH₃ (7 M Solution in MeOH)]=65/35 Flow rate: 80 g/min Wave length: UV 214 nm Temperature: 35° C.) to give (11S,12S)-12-(2-chlorophenyl)-11-cyclopropyl-7-fluoro-2,3,10-triazatricyclo[7.3.1.0\hat{ }{5,13}]trideca-1,5(13),6,8-tetraen-4-one (0207, 10.9 mg, 4%) as a white solid and (11R,12R)-12-(2-chlorophenyl)-11-cyclopropyl-7-fluoro-2,3,10-triazatricyclo[7.3.1.0\hat{ }{5,13}]trideca-1,5(13),6,8-tetraen-4-one (0207B, 41.4 mg, 14%) as a white solid.

0207A: MS (ESI): mass calcd. for C₁₈H₁₅ClFN₃O, 343.1, m/z found 344.1 [M+H]⁺. ¹H NMR (400 MHz, DMSO) δ 12.36 (s, 1H), 7.50-7.45 (m, 1H), 7.42-7.22 (m, 3H), 7.06-6.88 (m, 3H), 4.55 (d, J=6.8 Hz, 1H), 2.91-2.83 (m, 1H), 1.10-0.94 (m, 1H), 0.55-0.22 (m, 3H), 0.06-−0.10 (m, 1H).

0207B: MS (ESI): mass calcd. for C₁₈H₁₅ClFN₃O, 343.1, m/z found 344.1 [M+H]⁺. ¹H NMR (400 MHz, DMSO) δ 12.37 (s, 1H), 7.52-7.45 (m, 1H), 7.36 (s, 1H), 7.31-7.16 (m, 2H), 7.09-6.78 (m, 3H), 4.55 (d, J=6.8 Hz, 1H), 2.95-2.82 (m, 1H), 1.11-0.90 (m, 1H), 0.53-0.26 (m, 3H), 0.04-−0.07 (m, 1H).

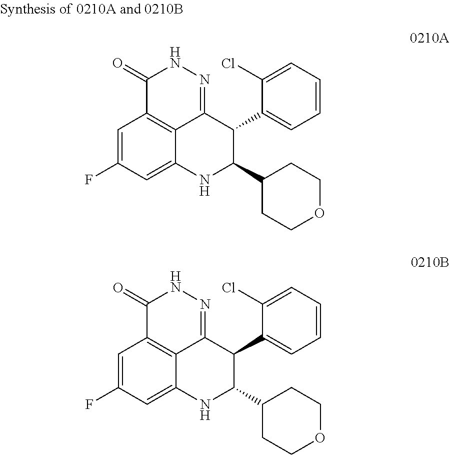 [SMILES: O=c1[nH]nc2c3c(cc(F)cc13)N[C@H](C1CCOCC1)[C@H]2c1ccccc1Cl ; O=c1[nH]nc2c3c(cc(F)cc13)N[C@@H](C1CCOCC1)[C@@H]2c1ccccc1Cl]

Step 1: To a solution of methyl 2-[2-(2-chlorophenyl)acetyl]-5-fluoro-3-nitrobenzoate (1.0 g, 2.84 mmol) in MeOH (4 mL) and THE (20 mL) were added oxane-4-carbaldehyde (649 mg, 5.69 mmol) and TiCl₃ (10.1 g, 17.06 mmol). The reaction mixture was stirred at 40° C. for 16 hours. The reaction mixture was concentrated under reduced pressure. The mixture was diluted with water (30 mL) and extracted with EA (50 mL×3). The organic layer was concentrated and the residue was purified by flash chromatograph on silica gel column (PE:EA=3:1) to afford methyl 3-(2-chlorophenyl)-7-fluoro-2-(oxan-4-yl)-4-oxo-2,3-dihydro-1H-quinoline-5-carboxylate (800 mg, 61%) as yellow solid. MS (ESI): mass calcd. for C₂₂H₂₁ClFNO₄, 417.1, m/z found 418.1 [M+H]⁺.

Step 2: To a mixture of methyl 3-(2-chlorophenyl)-7-fluoro-2-(oxan-4-yl)-4-oxo-2,3-dihydro-1H-quinoline-5-carboxylate (750 mg, 1.79 mmol) in MeOH (10 mL) was added N₂H₄·H₂O (173 mg, 5.38 mmol). The reaction mixture was stirred at 60° C. for 16 hours. The mixture was concentrated under reduced pressure to give a residue which was purified by reversed phase column (40% A in B; A: CH₃CN, B: 0.1% FA in water) to afford 12-(2-chlorophenyl)-7-fluoro-11-(oxan-4-yl)-2,3,10-triazatricyclo[7.3.1.0\hat{ }{5,13}]trideca-1,5(13),6,8-tetraen-4-one (300 mg, 40%) as yellow solid. MS (ESI): mass calcd. for C₂₁H₁₉ClFN₃O₂, 399.1, m/z found 400.1 [M+H]⁺.

Step 3: 12-(2-chlorophenyl)-7-fluoro-11-(oxan-4-yl)-2,3,10-triazatricyclo[7.3.1.0\hat{ }{5,13}]trideca-1,5(13),6,8-tetraen-4-one (140 mg, 0.35 mmol) was separated by Prep-SFC (Apparatus: SFC 150 Column: Daicel CHIRALPAK IA 4.6*250 mm, 5 um. Mobile phase: CO₂/MeOH [0.2% NH₃ (7 M Solution in MeOH)]=60/40 Flow rate: 80 g/min Wave length: UV 214 nm Temperature: 35° C.) to give (11S,12S)-12-(2-chlorophenyl)-7-fluoro-11-(oxan-4-yl)-2,3,10-triazatricyclo[7.3.1.0\hat{ }{5,13}]trideca-1,5(13),6,8-tetraen-4-one (0210, 17.5 mg, 13%) as white solid and (11R,12R)-12-(2-chlorophenyl)-7-fluoro-11-(oxan-4-yl)-2,3,10-triazatricyclo[7.3.1.0\hat{ }{5,13}]trideca-1,5(13),6,8-tetraen-4-one (0210B, 7 mg, 5%) as white solid.

0210A: MS (ESI): mass calcd. for C₂₁H₁₉ClFN₃O₂, 399.1, m/z found 400.1 [M+H]⁺. ¹H NMR (400 MHz, DMSO-d₆) δ 12.44 (s, 1H), 7.51 (d, J=8.0 Hz, 1H), 7.38 (d, J=2.8 Hz, 1H), 7.31-7.23 (m, 1H), 7.22-7.15 (m, 1H), 7.01 (dd, J=9.2, 2.4 Hz, 1H), 6.90 (dd, J=11.2, 2.4 Hz, 1H), 6.70-6.63 (m, 1H), 4.57 (d, J=3.2 Hz, 1H), 3.93-3.76 (m, 2H), 3.25-3.05 (m, 2H), 1.76-1.47 (m, 4H), 1.39-1.25 (m, 1H).

0210B: MS (ESI): mass calcd. for C₂₁H₁₉ClFN₃O₂, 399.1, m/z found 400.1 [M+H]⁺. ¹H NMR (400 MHz, DMSO-d₆) δ 12.45 (s, 1H), 7.51 (d, J=8.0 Hz, 1H), 7.40 (d, J=2.8 Hz, 1H), 7.30-7.23 (m, 1H), 7.22-7.15 (m, 1H), 7.01 (dd, J=9.2, 2.4 Hz, 1H), 6.90 (dd, J=11.2, 2.4 Hz, 1H), 6.69-6.63 (m, 1H), 4.57 (d, J=3.2 Hz, 1H), 3.92-3.78 (m, 2H), 3.26-3.06 (m, 2H), 1.75-1.47 (m, 4H), 1.44-1.17 (m, 1H).

### Additional Exemplary Compounds of The Present Technology

Additional compounds of the present technology were synthesized and isolated according to similar methods and procedures as described above, where such additional compounds include (but are not limited to):

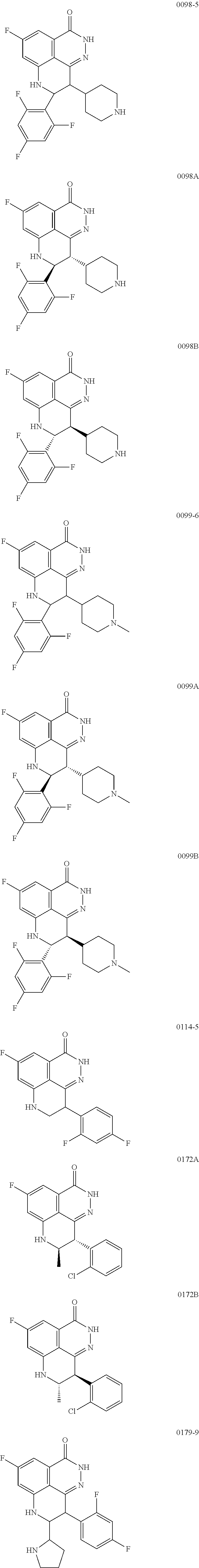

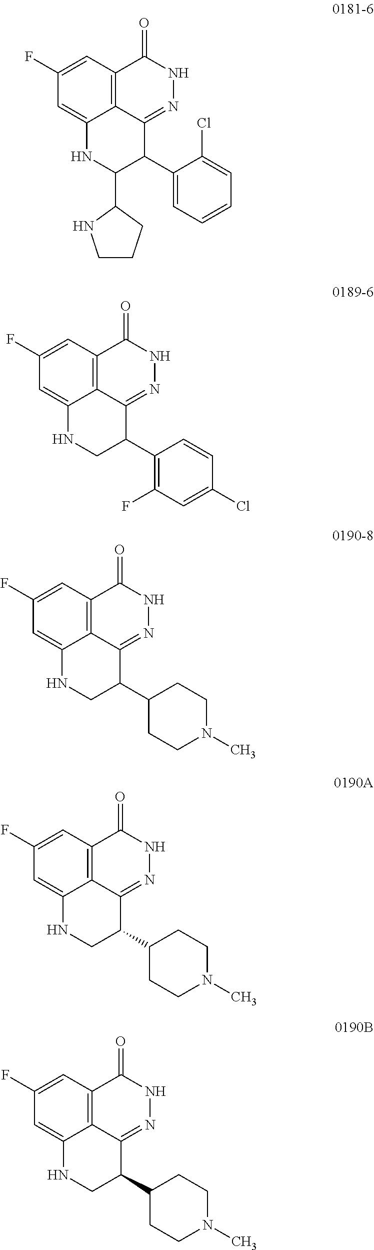

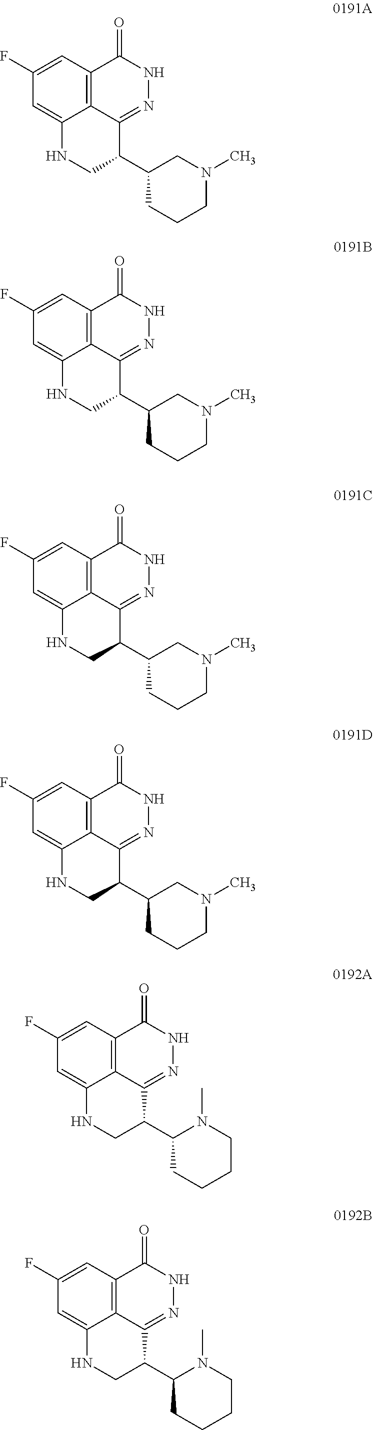 [SMILES: CN1CCC[C@H]([C@@H]2CNc3cc(F)cc4c(=O)[nH]nc2c34)C1 ; CN1CCC[C@@H]([C@@H]2CNc3cc(F)cc4c(=O)[nH]nc2c34)C1 ; CN1CCC[C@H]([C@H]2CNc3cc(F)cc4c(=O)[nH]nc2c34)C1 ; CN1CCC[C@@H]([C@H]2CNc3cc(F)cc4c(=O)[nH]nc2c34)C1 ; CN1CCCC[C@@H]1[C@@H]1CNc2cc(F)cc3c(=O)[nH]nc1c23 ; CN1CCCC[C@H]1[C@@H]1CNc2cc(F)cc3c(=O)[nH]nc1c23]

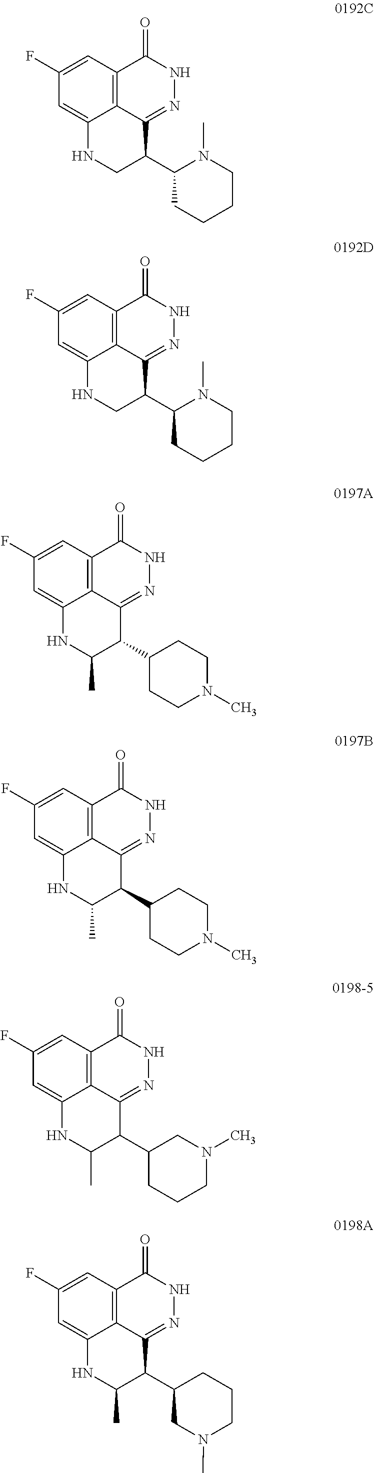 [SMILES: CN1CCCC[C@@H]1[C@H]1CNc2cc(F)cc3c(=O)[nH]nc1c23 ; CN1CCCC[C@H]1[C@H]1CNc2cc(F)cc3c(=O)[nH]nc1c23 ; C[C@H]1Nc2cc(F)cc3c(=O)[nH]nc(c23)[C@@H]1C1CCN(C)CC1 ; C[C@@H]1Nc2cc(F)cc3c(=O)[nH]nc(c23)[C@H]1C1CCN(C)CC1 ; CC1Nc2cc(F)cc3c(=O)[nH]nc(c23)C1C1CCCN(C)C1 ; C[C@H]1Nc2cc(F)cc3c(=O)[nH]nc(c23)[C@H]1[C@H]1CCCN(C)C1]

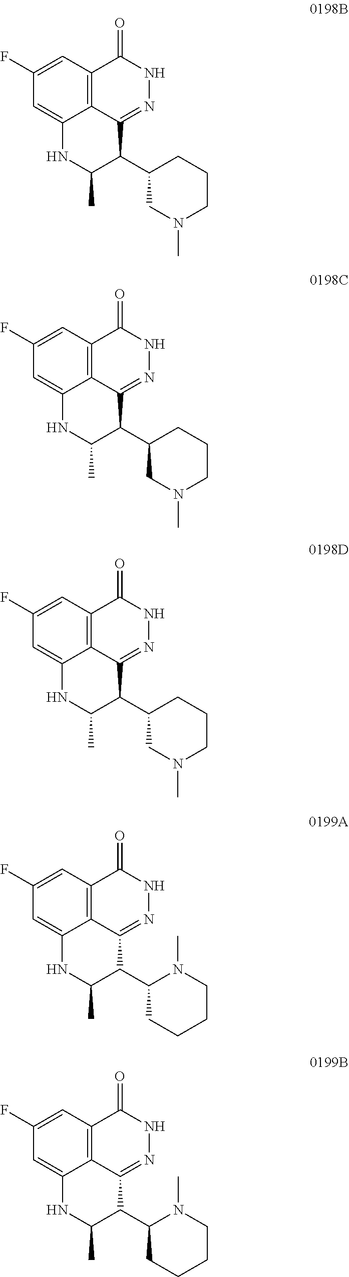

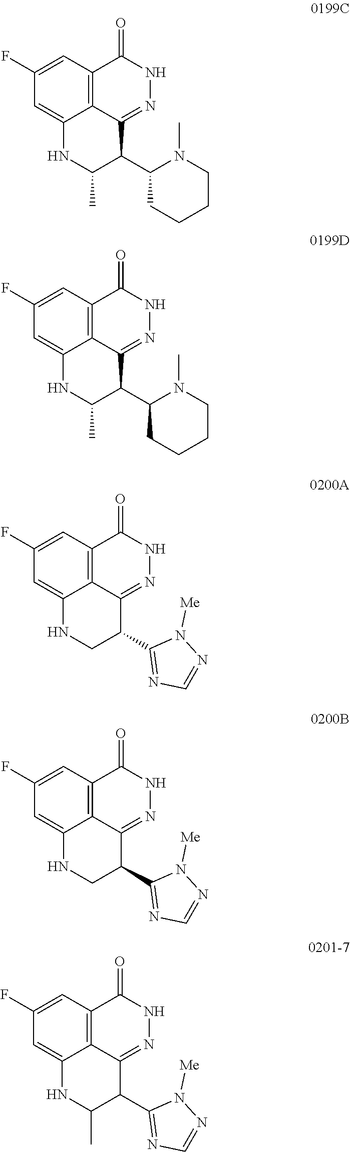 [SMILES: C[C@@H]1Nc2cc(F)cc3c(=O)[nH]nc(c23)[C@H]1[C@H]1CCCCN1C ; C[C@@H]1Nc2cc(F)cc3c(=O)[nH]nc(c23)[C@H]1[C@@H]1CCCCN1C ; Cn1ncnc1[C@@H]1CNc2cc(F)cc3c(=O)[nH]nc1c23 ; Cn1ncnc1[C@H]1CNc2cc(F)cc3c(=O)[nH]nc1c23 ; CC1Nc2cc(F)cc3c(=O)[nH]nc(c23)C1c1ncnn1C]

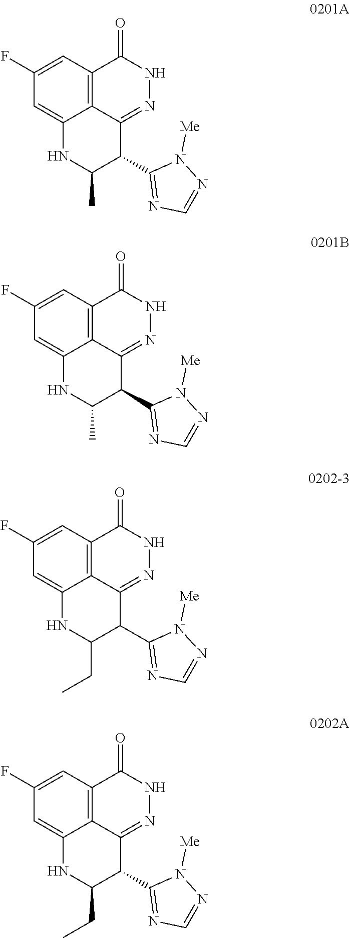 [SMILES: C[C@H]1Nc2cc(F)cc3c(=O)[nH]nc(c23)[C@@H]1c1ncnn1C ; C[C@@H]1Nc2cc(F)cc3c(=O)[nH]nc(c23)[C@H]1c1ncnn1C ; CCC1Nc2cc(F)cc3c(=O)[nH]nc(c23)C1c1ncnn1C ; CC[C@H]1Nc2cc(F)cc3c(=O)[nH]nc(c23)[C@@H]1c1ncnn1C]

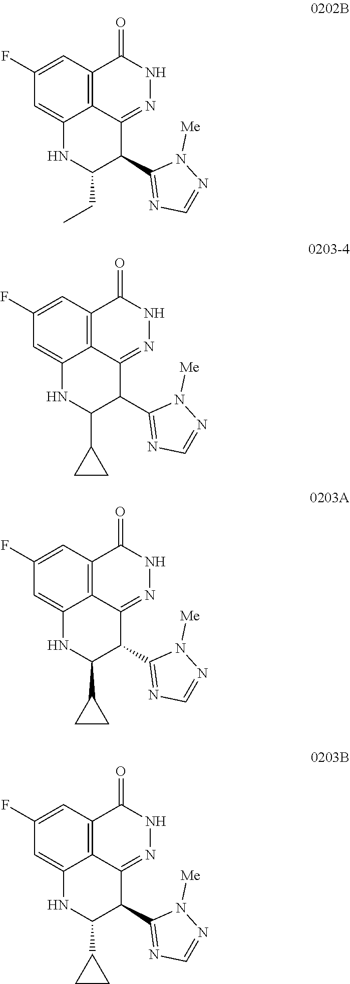

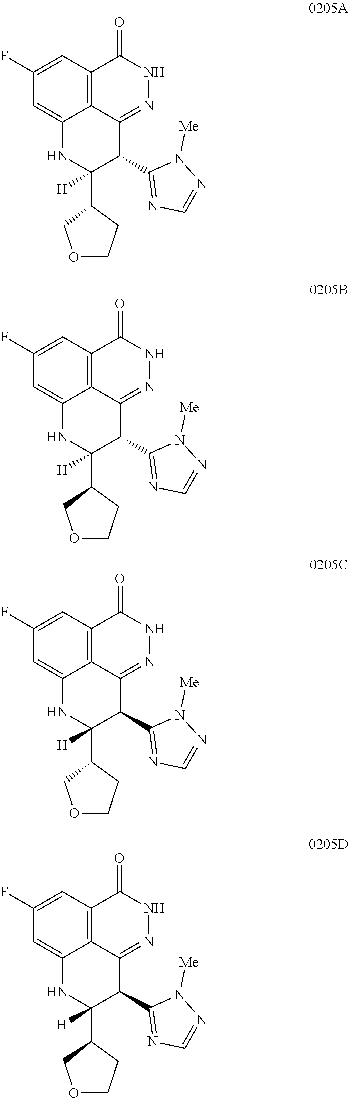

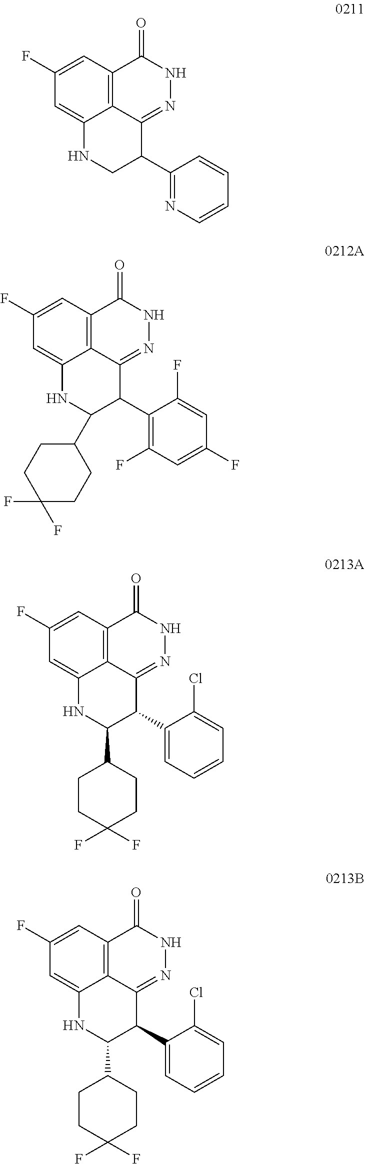 [SMILES: O=c1[nH]nc2c3c(cc(F)cc13)NCC2c1ccccn1 ; O=c1[nH]nc2c3c(cc(F)cc13)NC(C1CCC(F)(F)CC1)C2c1c(F)cc(F)cc1F ; O=c1[nH]nc2c3c(cc(F)cc13)N[C@H](C1CCC(F)(F)CC1)[C@H]2c1ccccc1Cl ; O=c1[nH]nc2c3c(cc(F)cc13)N[C@@H](C1CCC(F)(F)CC1)[C@@H]2c1ccccc1Cl]

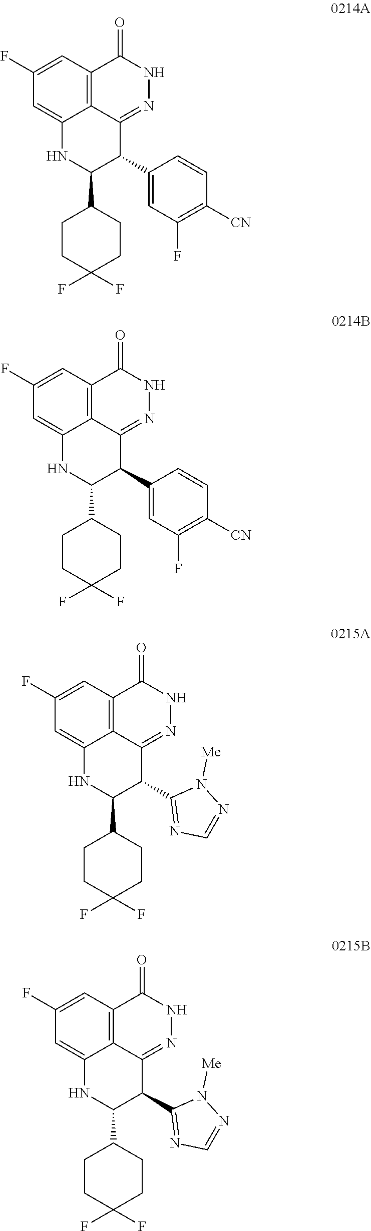

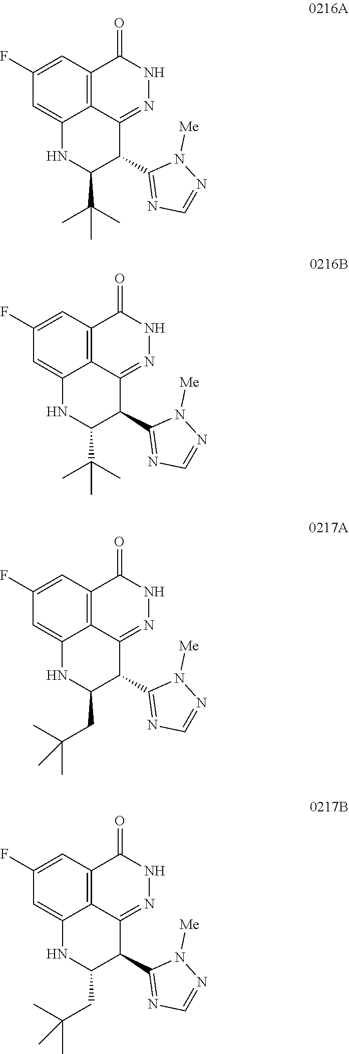 [SMILES: Cn1ncnc1[C@H]1c2n[nH]c(=O)c3cc(F)cc(c23)N[C@@H]1C(C)(C)C ; Cn1ncnc1[C@@H]1c2n[nH]c(=O)c3cc(F)cc(c23)N[C@H]1C(C)(C)C ; Cn1ncnc1[C@H]1c2n[nH]c(=O)c3cc(F)cc(c23)N[C@@H]1CC(C)(C)C ; Cn1ncnc1[C@@H]1c2n[nH]c(=O)c3cc(F)cc(c23)N[C@H]1CC(C)(C)C]

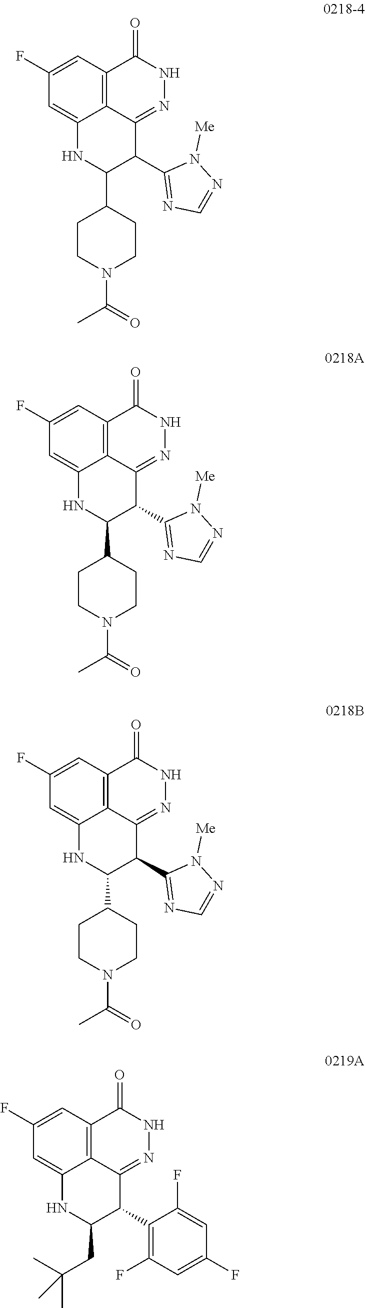 [SMILES: CC(=O)N1CCC(C2Nc3cc(F)cc4c(=O)[nH]nc(c34)C2c2ncnn2C)CC1 ; CC(=O)N1CCC([C@H]2Nc3cc(F)cc4c(=O)[nH]nc(c34)[C@@H]2c2ncnn2C)CC1 ; CC(=O)N1CCC([C@@H]2Nc3cc(F)cc4c(=O)[nH]nc(c34)[C@H]2c2ncnn2C)CC1 ; CC(C)(C)C[C@H]1Nc2cc(F)cc3c(=O)[nH]nc(c23)[C@@H]1c1c(F)cc(F)cc1F]

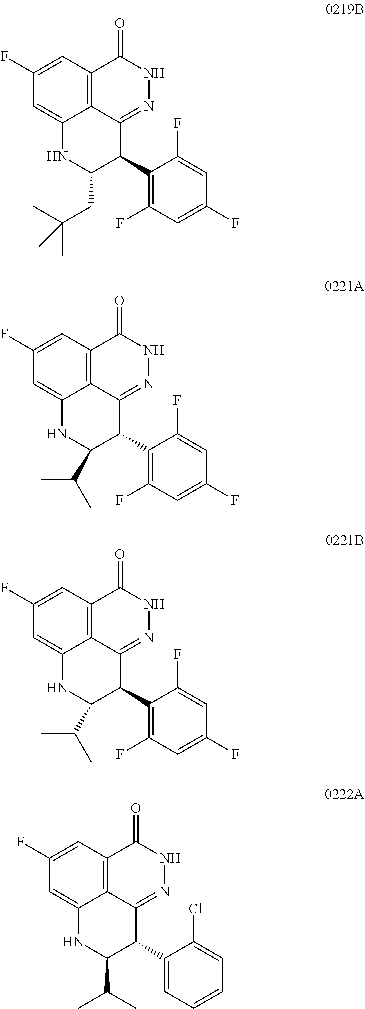 [SMILES: CC(C)(C)C[C@@H]1Nc2cc(F)cc3c(=O)[nH]nc(c23)[C@H]1c1c(F)cc(F)cc1F ; CC(C)[C@H]1Nc2cc(F)cc3c(=O)[nH]nc(c23)[C@@H]1c1c(F)cc(F)cc1F ; CC(C)[C@@H]1Nc2cc(F)cc3c(=O)[nH]nc(c23)[C@H]1c1c(F)cc(F)cc1F ; CC(C)[C@H]1Nc2cc(F)cc3c(=O)[nH]nc(c23)[C@@H]1c1ccccc1Cl]

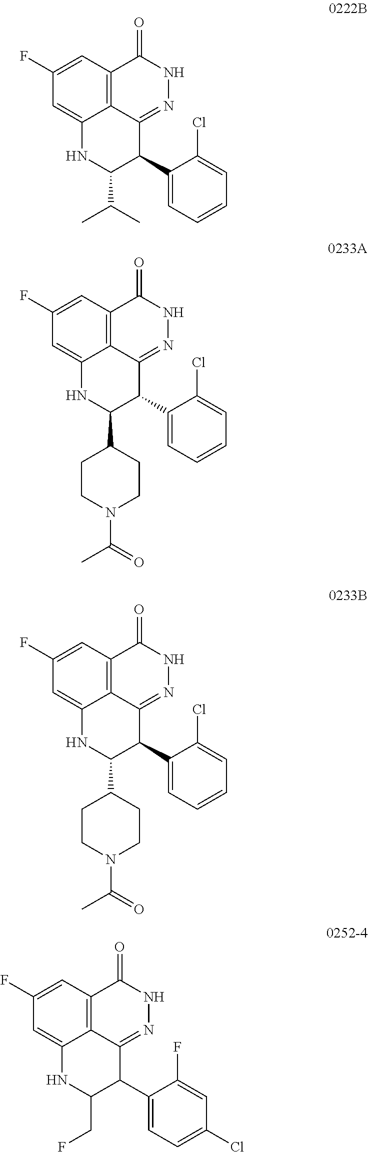

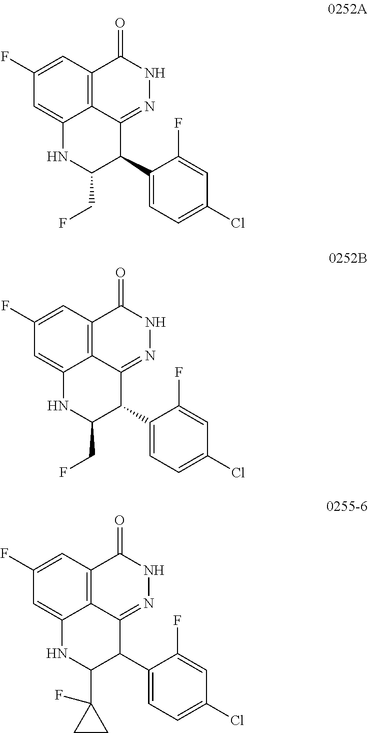 [SMILES: O=c1[nH]nc2c3c(cc(F)cc13)N[C@@H](CF)[C@@H]2c1ccc(Cl)cc1F ; O=c1[nH]nc2c3c(cc(F)cc13)N[C@H](CF)[C@H]2c1ccc(Cl)cc1F ; O=c1[nH]nc2c3c(cc(F)cc13)NC(C1(F)CC1)C2c1ccc(Cl)cc1F]

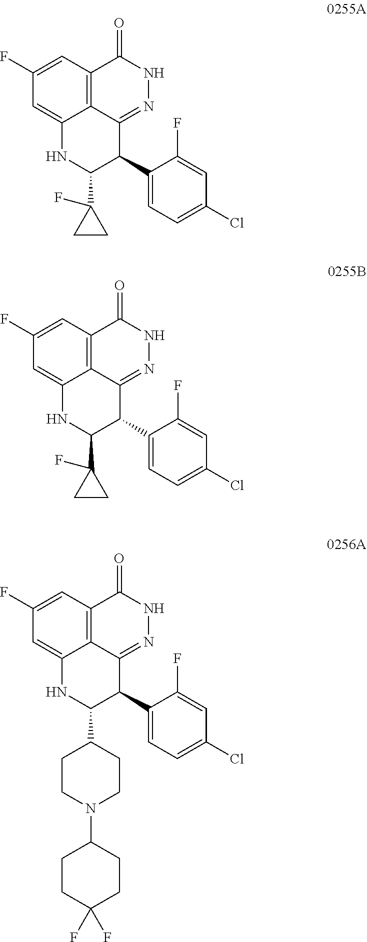 [SMILES: O=c1[nH]nc2c3c(cc(F)cc13)N[C@@H](C1(F)CC1)[C@@H]2c1ccc(Cl)cc1F ; O=c1[nH]nc2c3c(cc(F)cc13)N[C@H](C1(F)CC1)[C@H]2c1ccc(Cl)cc1F ; O=c1[nH]nc2c3c(cc(F)cc13)N[C@@H](C1CCN(C3CCC(F)(F)CC3)CC1)[C@@H]2c1ccc(Cl)cc1F]

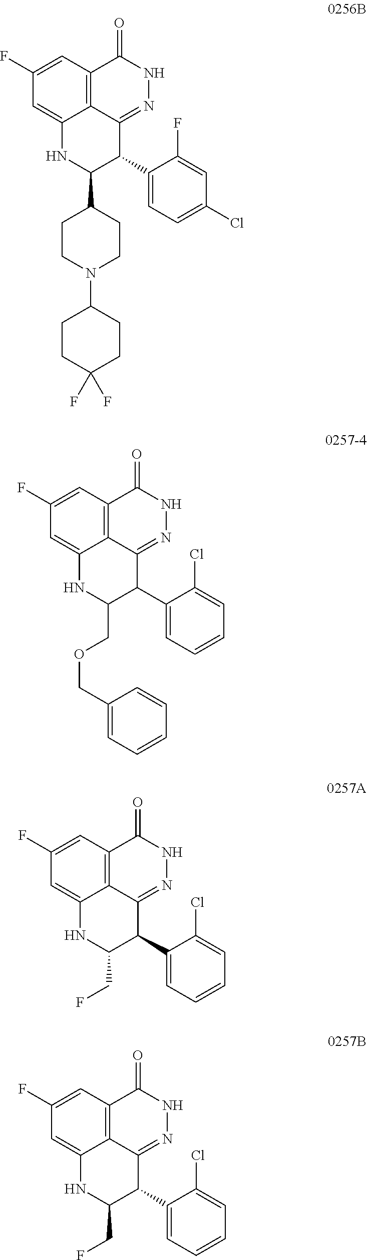 [SMILES: O=c1[nH]nc2c3c(cc(F)cc13)N[C@H](C1CCN(C3CCC(F)(F)CC3)CC1)[C@H]2c1ccc(Cl)cc1F ; O=c1[nH]nc2c3c(cc(F)cc13)NC(COCc1ccccc1)C2c1ccccc1Cl ; O=c1[nH]nc2c3c(cc(F)cc13)N[C@@H](CF)[C@@H]2c1ccccc1Cl ; O=c1[nH]nc2c3c(cc(F)cc13)N[C@H](CF)[C@H]2c1ccccc1Cl]

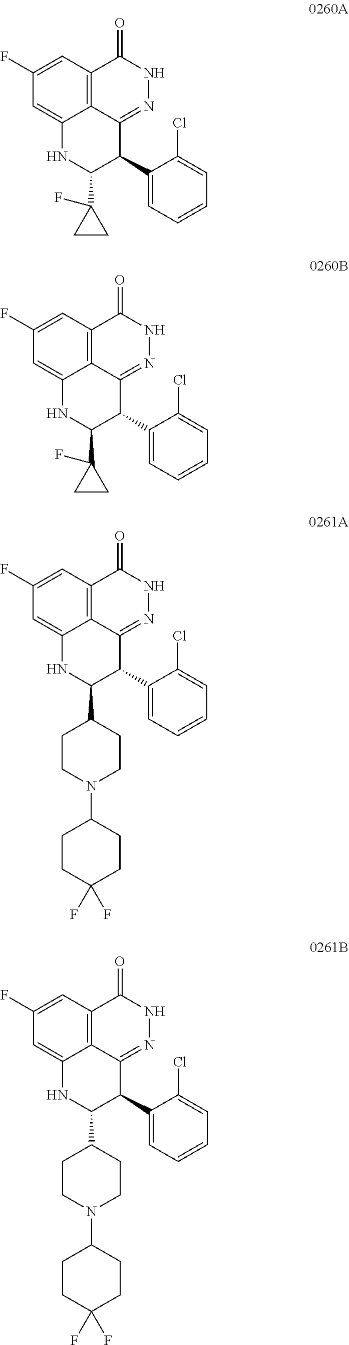

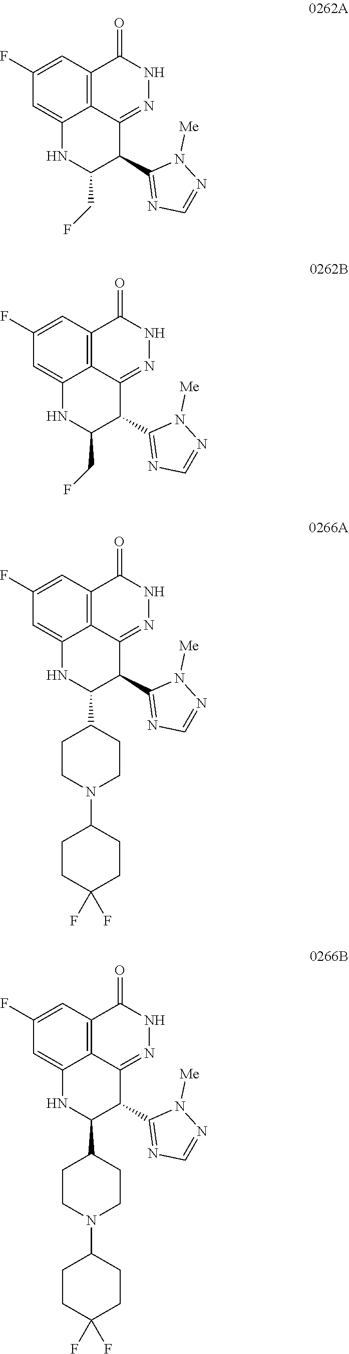 [SMILES: Cn1ncnc1[C@@H]1c2n[nH]c(=O)c3cc(F)cc(c23)N[C@H]1CF ; Cn1ncnc1[C@H]1c2n[nH]c(=O)c3cc(F)cc(c23)N[C@@H]1CF ; Cn1ncnc1[C@@H]1c2n[nH]c(=O)c3cc(F)cc(c23)N[C@H]1C1CCN(C2CCC(F)(F)CC2)CC1 ; Cn1ncnc1[C@H]1c2n[nH]c(=O)c3cc(F)cc(c23)N[C@@H]1C1CCN(C2CCC(F)(F)CC2)CC1]

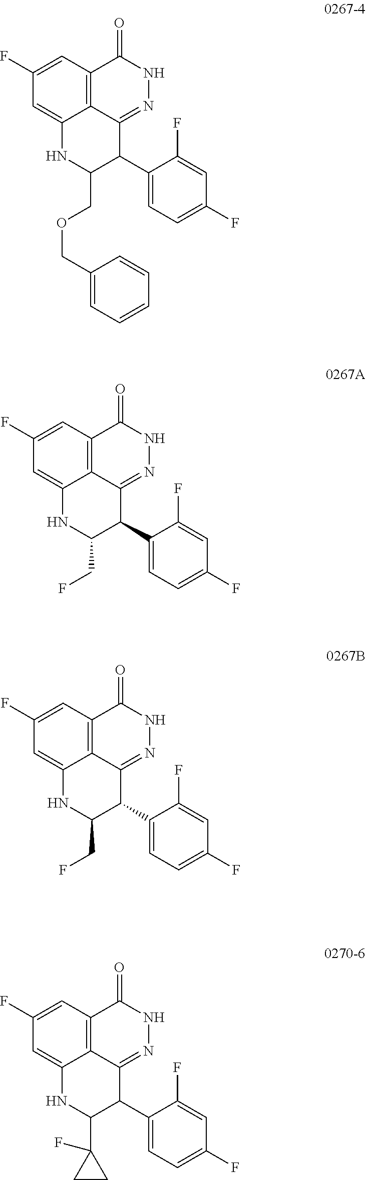 [SMILES: O=c1[nH]nc2c3c(cc(F)cc13)NC(COCc1ccccc1)C2c1ccc(F)cc1F ; O=c1[nH]nc2c3c(cc(F)cc13)N[C@@H](CF)[C@@H]2c1ccc(F)cc1F ; O=c1[nH]nc2c3c(cc(F)cc13)N[C@H](CF)[C@H]2c1ccc(F)cc1F ; O=c1[nH]nc2c3c(cc(F)cc13)NC(C1(F)CC1)C2c1ccc(F)cc1F]

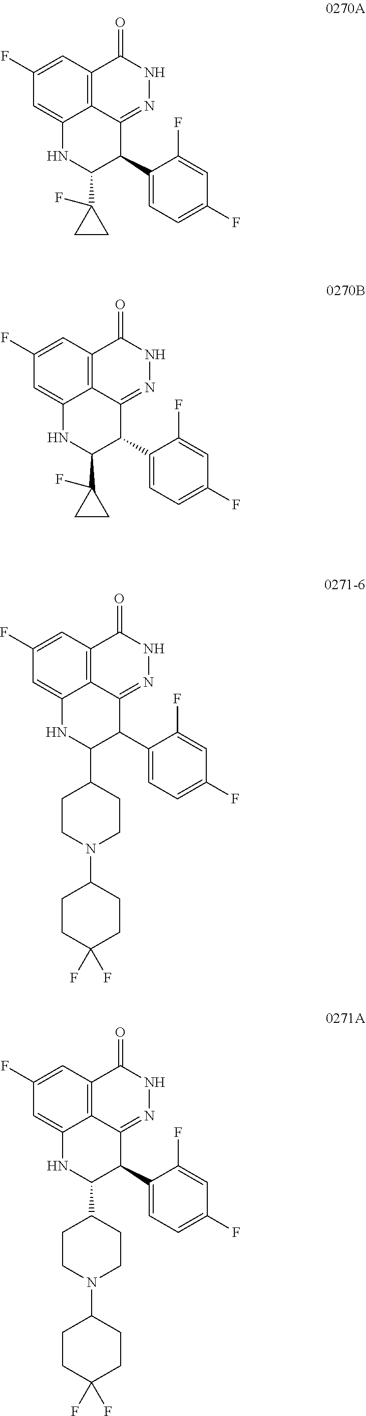 [SMILES: O=c1[nH]nc2c3c(cc(F)cc13)N[C@@H](C1(F)CC1)[C@@H]2c1ccc(F)cc1F ; O=c1[nH]nc2c3c(cc(F)cc13)N[C@H](C1(F)CC1)[C@H]2c1ccc(F)cc1F ; O=c1[nH]nc2c3c(cc(F)cc13)NC(C1CCN(C3CCC(F)(F)CC3)CC1)C2c1ccc(F)cc1F ; O=c1[nH]nc2c3c(cc(F)cc13)N[C@@H](C1CCN(C3CCC(F)(F)CC3)CC1)[C@@H]2c1ccc(F)cc1F]

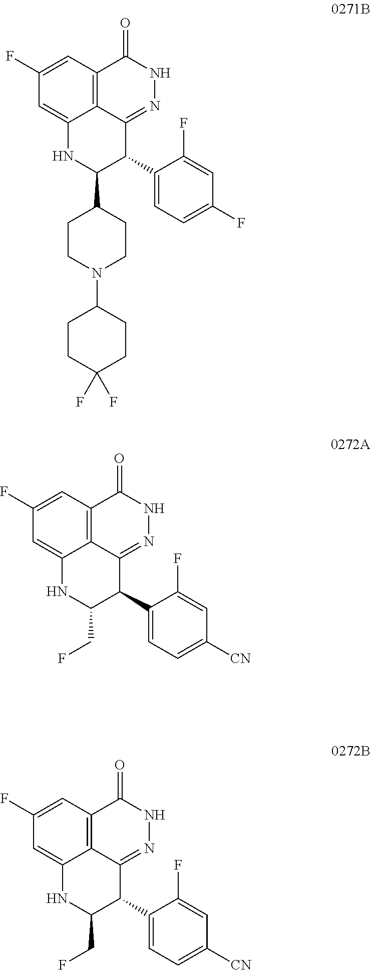 [SMILES: O=c1[nH]nc2c3c(cc(F)cc13)N[C@H](C1CCN(C3CCC(F)(F)CC3)CC1)[C@H]2c1ccc(F)cc1F ; N#Cc1ccc([C@@H]2c3n[nH]c(=O)c4cc(F)cc(c34)N[C@H]2CF)c(F)c1 ; N#Cc1ccc([C@H]2c3n[nH]c(=O)c4cc(F)cc(c34)N[C@@H]2CF)c(F)c1]

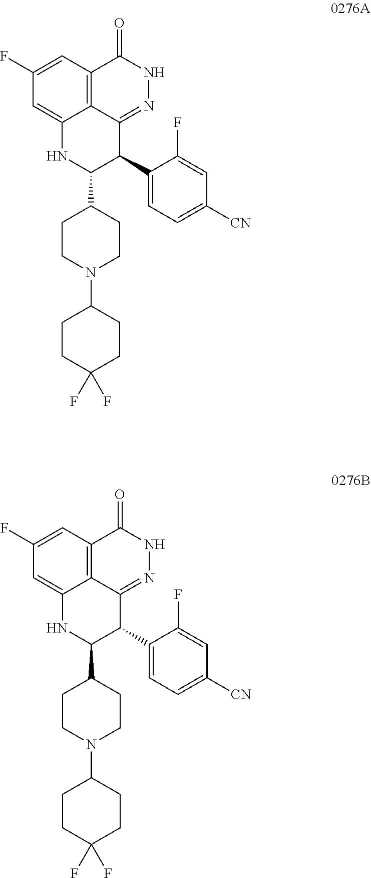 [SMILES: N#Cc1ccc([C@@H]2c3n[nH]c(=O)c4cc(F)cc(c34)N[C@H]2C2CCN(C3CCC(F)(F)CC3)CC2)c(F)c1 ; N#Cc1ccc([C@H]2c3n[nH]c(=O)c4cc(F)cc(c34)N[C@@H]2C2CCN(C3CCC(F)(F)CC3)CC2)c(F)c1]

### PARP Mass Spectroscopy Assay Protocol

Materials and Reagents: The PARP1 enzyme was purchased from BPS Bioscience (cat #80501). Tris-HCl, pH 8.0 was purchased from Corning (cat #46-031-CM). Magnesium chloride was purchased from Thermo Fisher Scientific (previously Honeywell Fluka, cat #63020-1L). All other assay components, activated DNA (cat #D4522), core histones (cat #SRP6590), β-Nicotinamide adenine dinucleotide (β-NAD—cat #N8285), 3ABA PARP1 small molecule inhibitor (from the PARP1 Enzyme Activity Assay kit, cat #17-10149), Sodium Chloride (NaCl, cat #56546-1L), Triton X-100 (cat #93443), Dithiothreitol (DTT, cat #43816-250 ML) were all purchased from Millipore Sigma.

Assay Buffer: The assay buffer includes the following reagents: 50 mM Tris-HCl pH 8.0, 50 mM NaCl, 10 mM, MgCl2, 0.01% Triton X-100, and 1 mM DTT.

### Procedure:

PARP1 enzymatic assays were performed in 384-well plates in a total volume of 20 μL in the assay buffer. For concentration response curves, compounds were serially diluted 3-fold from 2 mM highest concentration 0.1013 mM to generate a 10-point curve, and 125 nL was transferred to the assay plate using the Echo acoustic dispenser for a final concentration range of 26.6 μM to 1.35 μM in 10 μL reaction. Five microliters of 10 nM PARP1 enzyme and 100 nM core histones (2×) mix were added to the assay plates and pre-incubated for 30 mins at room temperature (RT). The reaction was initiated by the addition of a 5 μL of 10 uM β-NAD plus 0.02 mg/mL activated DNA (2×) mix. The reaction is incubated for 60 min at RT. The final concentration of the PARP1 enzyme and substrate concentrations were 5 nM and 5 uM, respectively. The positive control (high signal) and negative control (low signal) wells had 125 nL of DMSO in place of compounds. After the incubation is complete, the reaction is stopped by the addition of 10 μL of 10 uM of the 3ABA PARP1 inhibitor, incubated for 5 min at RT, and placed in the −80 C freezer for shipment to the Valo Health site in Branford Connecticut where mass spectrometry will be used to directly detect quantities of nicotinamide (NAM).

The percent inhibition of enzyme activity was calculated according to the following equation:

$$\% Inhibition = ( 1 - ( \frac{{S}_{sample} - {S}_{low}}{{S}_{high} - {S}_{low}} ) ) \times 100.$$

The values of Sₛₐₘₚₗₑ, S_{high}, and S_{low} in the equation refer to the NAM concentration detected in the assay wells, high control wells, and low control wells, respectively. To determine inhibitor IC₅₀ values, % inhibition of the CRCs (concentration response curves) was fitted to the standard single-site four-parameter logistic equation.

### Representative Results for Exemplary Compounds Of the Present Technology

Table 1 provides representative initial results for exemplary compounds of the present technology.

TABLE 1

| Compound | PARP1 IC₅₀ (μM) |
|---|---|
| 0260A | 0.002 |
| 0194A | 0.002 |
| 0221A | 0.002 |
| 0255-6 | 0.004 |
| 0271-6 | 0.004 |
| 0255A | 0.004 |
| 0207B | 0.006 |
| 0206B | 0.006 |
| 0271B | 0.007 |
| 0252B | 0.008 |
| 0174B | 0.008 |
| 0196-5 | 0.009 |
| 0187A | 0.009 |
| 0223B | 0.009 |
| 0206A | 0.009 |
| 0196 | 0.009 |
| 0193B | 0.009 |
| 0115A | 0.010 |
| 0172A | 0.010 |
| 0092A; 0092B | 0.013 |
| 0267-4 | 0.013 |
| 0212A | 0.015 |
| 0195-5 | 0.018 |
| 0222B | 0.018 |
| 0172B | 0.018 |
| 0221B | 0.019 |
| 0214B | 0.020 |
| 0212A | 0.020 |
| 0195A | 0.021 |
| 0192B | 0.022 |
| 0222A | 0.023 |
| 0193A | 0.024 |
| 0199D | 0.025 |
| 0195B | 0.026 |
| 0211A | 0.027 |
| 0192A | 0.027 |
| 0196B | 0.027 |
| 0188A | 0.028 |
| 0189B | 0.029 |
| 0276B | 0.030 |
| 0098B | 0.030 |
| 0121A | 0.032 |
| 0114-5 | 0.032 |
| 0191D | 0.035 |
| 0194B | 0.035 |
| 0174A | 0.036 |
| 0191B | 0.037 |
| 0191A | 0.037 |
| 0199C | 0.038 |
| 0121-4 | 0.039 |
| 0198B | 0.039 |
| 0187B | 0.041 |
| 0186B | 0.041 |
| 0114B | 0.041 |
| 0189-6 | 0.041 |
| 0198D | 0.042 |
| 0188-5 | 0.042 |
| 0121D | 0.043 |
| 0198C | 0.045 |
| 0110B | 0.045 |
| 0191-8 | 0.046 |
| 0120B | 0.047 |
| 0219B | 0.049 |
| 0191C | 0.050 |
| 0111A | 0.051 |
| 0252A | 0.057 |
| 0207A | 0.064 |
| 0199A | 0.065 |
| 0173A | 0.070 |
| 0189A | 0.079 |
| 0111B | 0.080 |
| 0117C | 0.085 |
| 0120 | 0.087 |
| 0171 | 0.091 |
| 0098A | 0.098 |
| 0188B | 0.099 |
| 0117A | 0.101 |
| 0255B | 0.103 |
| 0260B | 0.106 |
| 0210A | 0.106 |
| 0214A | 0.108 |
| 0199B | 0.118 |
| 0111C | 0.120 |
| 0116B | 0.123 |
| 0213B | 0.130 |
| 0210B | 0.142 |
| 0111-6 | 0.164 |
| 0115B | 0.190 |
| 0121B | 0.236 |
| 0186A | 0.243 |
| 0198-5 | 0.244 |
| 0115C | 0.252 |
| 0114A | 0.270 |
| 0276A | 0.281 |
| 0117B | 0.287 |
| 0261B | 0.301 |
| 0213A | 0.352 |
| 0212B; 0215-4 | 0.438 |
| 0197A | 0.447 |
| 0219A | 0.512 |
| 0117-8 | 0.566 |
| 0257-4 | 0.593 |
| 0121C | 0.854 |
| 0261A | 0.896 |
| 0271A | 0.936 |
| 0223A | 1.200 |
| 0181-6 | 1.200 |
| 0111D | 1.240 |
| 0110A | 1.40 |
| 0266A | 1.680 |
| 0117D | 3.080 |
| 0116A | 5.280 |
| 0266B | 5.780 |

### PARylation Immunofluorescence Assay Protocol

Materials:

| Reagent/Consumable | Supplier | Cat No. |
|---|---|---|
| HCT116 | ATCC | CCL-247 ™ |
| McCoys 5A Media | ATCC | 30-2007 |
| Fetal Bovine Serum | Corning | 35-010-CV |
| L-Glutamine | Sigma | 59202C |
| Solution 200 mM | | |
| Antibiotic antimycotic | Sigma | A5955-100 ML |
| Solution (100X) | | |
| Corning ® 384-well Flat | Corning | 3764BC |
| Clear Bottom Black | | |
| Polystyrene TC- | | |
| treated Microplates | | |
| Echo ® Qualified 384-Well | Labcyte | LP-0200 |
| COC Source Microplate | | |
| 100% methanol | Sigma | 646377-4X4L |
| Tween 20 | Sigma | P9416-100 ML |
| Triton X-100 (10%) | Sigma-Aldrich | 93443 |
| DPBS | Gibco, Thermo/Sigma | 14040141/ |
| | | D8662-6X1L |
| Hoechst HO33342 | Life Technologies | H3570 |
| Roche Blocking Reagent | Roche | 11-096-176-001 |
| Anti-PAR (Ab-1) | Sigma Millipore | AM80 |
| Mouse mAb (10H) | | |
| Anti-Mouse | CST | 44085 |
| Secondary, AF488 | | |
| METHYL | Sigma | 129925-5G |
| METHANESULFONATE | | |
| (MMS) | | |

Equipment:

| Equipment Name | Vendor |
|---|---|
| Multidrop Combi | ThermoFisher |
| Echo 555 | Labcyte/Beckman Coulter Life Sciences |
| Cellometer Auto T4 Bright | Nexcelom Bioscience |
| Field Cell Counter | |
| Bluewasher | Blue Cat Bio |
| PlateLoc Thermal | Agilent |
| Microplate Sealer | |
| CX7 | Thermofisher |

Cell Culture Details: HCT116 is a human colon epithelial cell line, which grows adherently in tissue culture flasks. Cells are grown in T175-sized flasks. Split two times a week, at 70-80% confluence, varying from 1:5-1:10 splits, following standard adherent cell subculturing protocols. Media is kept refrigerated until day of cell-based activities and needs to be warmed to at least RT prior to usage. Cell Culture Media: McCoy's 5A with 100% FBS, 2 mM L-Glutamine, 20 mM HEPES. Plating Media: McCoy's 5A with 100% FBS, 2 mM L-Glutamine, 20 mM HEPES, 100 Antibiotic-Antimycotic solution.

### Procedure:

Day 1: Cell Culture and Cell Plating: The cell culture and cell plating methods include the following steps: Warm media prior to cell culture; remove flask of cells from incubator and trypsinize cells in T175 flask using 0.25% Trypsin; return the flask to the incubator for ˜5 minutes to allow the cells to fall away from the bottom of the flask; add 10 uL of media to rinse bottom of the flask and add cells and media to a 50 mL conical tube; spin cells at 1000 rpm for 5 min, aspirate out media, and resuspend pellet with 10 mL fresh media; using the Cellometer Cell Counter, calculate the live cell count; using the live cell count, and the number of plates needed, calculate the amount of resuspended cell pellet media needed to add to fresh plating media to dispense 5 uL/well at the density of 6000 cell/well into the 384W plates; plate cells in 384W plates, 5 μL of media in each well at 6000 cells/well for the entire plate; gently shake plates (100 rpm) for 20 min after plating; and incubate plates at 37° C., 5% CO₂ overnight prior to treatment.

Day 2: Compound and DNA damage treatment, fixation and primary staining: The methods include the following steps: Using the Labcyte Echo 555, treat the 384W cell seeded plates to achieve a top dose of 10 uM for each compound. 40 nL of 10 mM top dose compound plates are stamped into the 40 μL of cells in the seeded plates. The Labcyte compound source plates are DMSO-based with ten-point, threefold dose dilutions of compounds, starting at 10 mM. The transfer volume using the Echo was 40 nL; incubate for 5 hours at 37° C., 5% CO₂; add DNA damaging agent MMS to columns 1-23 of each cell seeded 384W plate by delivering 40 nl of 19.2% MMS in DMSO using Echo in DMSO delivery mode, wherein the MMS stock concentration is 99% and must be diluted 1:5 in DMSO prior to delivery to cell plates; incubate cell plates at 37° C., 5% CO₂ for 30 minutes; evacuate media and add 75 ul of cold methanol using BlueWasher, wherein the Bluewasher MagBeadSpeed setting for all evacuations is: Spins plate 5 sec clock-wise at 800 RPM (35 g); keep the dispense line and the methanol, chilled throughout fixation process, and use the staccato setting to break dispense into multiple squirts to minimize cell disruption; incubate fixed plates for 20 mins. on ice; wash full plate 1× with equal volume of cold DPBS and evacuate with Bluewasher at MagBeadSpeed; add 20 ul of DPBS 0.1% Triton X-100 to full plate and incubate 15 min at room temp and evacuate with Bluewasher at MagBeadSpeed; add 20 ul of Roche block to the full plate and incubate for 60 minutes at room temperature and evacuate with Bluewasher at MagBeadSpeed; and add 20 ul of PAR antibody (1:4,000) in Roche block, seal plate and incubate overnight at 4 C.

Day 3: Secondary Antibody Staining and Imaging: The methods include the following steps: Evacuate and wash plates (3×) with 25-30 ul of DPBS 0.05% Tween 20 using the Bluewasher; add 20 ul of anti-mouse AF488 secondary antibody (1:1,600) plus Hoechst (1:10,000) and incubate for 60 min at room temperature; evacuate using MagBeadSpeed and wash 3-4× with 25-30 ul of DPBS 0.05% Tween 20; add 30 ul DPBS and seal plates; and image with CX7—Circle (Nuclear) Mean Average Intensity Channel 1 (360) and Channel 2 (488) using Protocol ‘PAR_HCT116_MeOH_10×_2ChR’. Key acquisition settings: exposure of 488 channels to 70-80% and 1-2 fields.

Data analysis: Raw data files are exported from the CX7 software, paired with barcoded compound plates to track compound ID, dose responses and Echo transfer records. This allows for import and QC analysis of dose response curves, and IC50 values determination. Images of each plate and well on both imaged channels can be exported as well. The percent inhibition of PARylation activity was calculated according to the following equation:

$$\% Inhibition = ( 1 - ( \frac{{S}_{sample} - {S}_{low}}{{S}_{high} - {S}_{low}} ) ) \times 100.$$

The values of Sₛₐₘₚₗₑ, S_{high}, and S_{low} in the equation refer to the PARylation levels detected in the assay wells, high control wells, and low control wells, respectively. To determine inhibitor IC₅₀ values, % inhibition of the CRCs (concentration response curves) was fitted to the standard single-site four-parameter logistic equation.

Assay Principle: Cellular levels of PARylation of PARP1/2 substrate proteins (PARP1 and histones) are measured with an anti-PAR antibody using an immunofluorescence assay upon DNA damage based on https://f1000research.com/articles/5-736/v2. PARP inhibitors, when co-treated with the DNA damaging agent MMS, reduce the levels of PARylation.

### Cell-Based Proliferation Assay Protocol: DLD1 Parental Vs BRCA2 Null 5 Day CTG, CTF, CyQuant, or One Pot Live-Dead HCS Assays

Reagents and consumables:

| Reagent/Consumable | Supplier | Cat No. |
|---|---|---|
| RPMI w/no phenol red | ThermoFisher | 11835030 |
| RPMI 1640 | Sigma | R8758 |
| FBS | Corning | 35-010-CV |
| Antibiotic- | Corning | 30004CI |
| Antimycotic solution | | |
| Trypsin/EDTA (0.25%) | Corning | 25-052-Cl |
| Echo Compound Plates | Labcyte | LP-0200 |
| 1536 well plates | Corning | 9094BC |
| 1536 well plates | Aurora Microplates | EBC2-01101A |
| 384 well plates | Corning | 3764BC |
| DLD1 parental | ATCC | ATCC ® CCL-221 ™ |
| DLD1 BRCA2+/− | Horizon | Horizon: HD105-027 |
| DLD1 BRCA2−/− | Horizon | Horizon: HD105-007 |
| CellTiter-Glo ® 2.0 Cell | Promega | G9243 |
| Viability Assay | | |
| CellTiter-Fluor ™ Cell | Promega | G6082 |
| Viability Assay | | |
| CyQUANT NF Cell | ThermoFisher | C35012 |
| Proliferation Assay Kit | | |
| Propidium Iodide | Life Technologies | R37169 |
| Ready Flow Reagent | | |
| Hoechst HO33342 | Life Technologies | H3570 |

Equipment:

| Equipment Name | Vendor |
|---|---|
| Multidrop Combi | ThermoFisher |
| Echo 555 | Labcyte/Beckman Coulter Life Sciences |
| Cellometer Auto T4 Bright | Nexcelom Bioscience |
| Field Cell Counter | |
| Bluewasher | Blue Cat Bio |
| PlateLoc Thermal | Agilent |
| Microplate Sealer | |
| CX7 | ThermoFisher |
| PHERAstar FS | BMG Labtech |
| EnVision | Perkin Elmer |

### Procedure

Day 1: Cell Culture and Cell Plating: The method includes the following steps: count cells (using Nexcelom cellometer and record cell count); spin cells at 1000 rpm for 5 min and re-suspend pellet with fresh media; and plate cells in 1536 well plate at specific cells/well density based on density optimization testin (or DLD1 parental (historically) 100 cell/well; for DLD1 BRCA2+/−vs. −/− null is 125 cell/well; uL of cells/well in columns 1-47 for 1536w plates); gently shake plates (100 rpm) for 30 min at room temperature before transfer to incubator (37° C., 5% CO₂); and incubate plates (37° C., 5% CO₂) for 24 h prior to treatment.

Day 2: Compound Treatment: For 1536w, compound stamping was 25 nl of 10 mM on the top concentration of the source plate compounds, resulting in 50 uM top dose concentration used for cell-based proliferation assay. Positive control SAHA is stamped to 10 uM final concentration

Days 3-7: Plate Incubation: Plates are left to incubate at 37° C., 5% CO₂ for a total of 120 h/5 days after compound addition.

Days 3-7: Readout Reagent Addition and Plate Reading: After incubation, plates are removed from the incubator, and the designated read out reagent is added to the cell plates. See below for details based on the specific readout.
CTG Readout: Remove CTG 2.0 reagent from the fridge and bring to room temperature prior to use; add 4 uL/well CTG to all wells of each cell plate; incubate plates at 37° C., 5% CO₂, 30 minutes for 1536w plates; and read plates using luminescence-specific protocol on designated plate readers; BMGs or Envisions have CTG-specific protocols.
CyQUANT Readout: Remove Cyquant Direct Cell Proliferation Assay Kit from the fridge and bring to room temperature prior to use. The reagents may need to be heated up in a drybath to thaw; calculate the total volume required based on well dispensed to ensure enough detection reagent is needed. Make up the detection reagent by combining the following assay recipe: PBS: 11.7 mL; CyQuant® Direct nucleic acid stain: 48 μL; and CyQuant® Direct background suppressor: 240 μL; add 2 ul/well Cyquant reagent mix to all wells; incubate plates at 37° C., 5% CO₂, for 60 minutes; read plates using CyQuant-specific protocol on designated plate readers BMGs or EnVisions with specific 1536w protocol (CyQUANTDirect—508/527 nm; CyQUANTDirect Red—622/645 nm).
CTF Readout: Remove CTF reagent from the freezer and bring to room temperature prior to use, prepare 1× reagent and vortex to dissolve (reagent stable at RT for 24 h or at 4 C for 7 days); add 4 ul/well CTF to all wells and incubate plates at 37° C., 5% CO₂, 180 minutes (3 h); and read plates using fluorescence-specific protocol on designated plate readers (BMGs or Envisions have CTF-specific protocols (Ex 380-400; Em 505)).
One Pot Live-Dead HCS Readout: After incubation, plates are removed from the incubator; for 1536 well plates, make 4 mL 1×PBS and add 8 drops of Propidium Iodide reagent+4 μL of Hoechst 333242; mix and then using a Multidrop Combi dispense 1 uL/well with medium speed; for 384 well plates: make 6 mL 1×PBS and add 12 drops of Propidium Iodide reagent+6 μL of Hoechst 333242, mix and then using a Multidrop Combi dispense 8 uL/well with medium speed; incubate for 30 minutes at 37° C., 5% CO₂; seal the plates with an aluminum seal; spin for 2 minutes at 1000 RPM in a spin bucket centrifuge; load plate onto CX7; and acquire data.
   Analyzed data is then exported as spot fire file and then processed in ABASE.

Data analysis for CTG, CyQUANT and CTF readouts: Raw data files are exported from the BMG, Envision and CX7 readers, paired with barcoded compound plates to track compound ID, dose responses and Echo transfer records. This allows for import and QC analysis of dose response curves, and IC50 values determination. Data and images of each plate and well on both imaged channels can be exported as well. The percent inhibition of growth activity was calculated according to the following equation:

$$\% Inhibition = ( 1 - ( \frac{{S}_{sample} - {S}_{low}}{{S}_{high} - {S}_{low}} ) ) \times 100.$$

The values of Sₛₐₘₚₗₑ, S_{high}, and S_{low} in the equation refer to the assay signals detected in the wells, high control wells, and low control wells, respectively. To determine inhibitor IC₅₀ values, % inhibition of the CRCs (concentration response curves) was fitted to the standard single-site four-parameter logistic equation.

Assay Principle: Cell viability is measured with assays that detect live-cell readouts: intracellular ATP (CTG), DNA (CyQUANT) and peptidase activity with cell permeant substrate Gly-Phe-AFC (CTF) using luminescence or fluorescence assays upon cell proliferation in the presence of PARP inhibitor compounds

### MDCK-MDR1 Permeability Assay Results for Exemplary Compounds of the Present Technology

The bi-directional assay containing a Mandin Darby canine kidney (MDCK) cell line transfected with human MDR1 gene (P-glycoprotein; P-gp) designed to overexpress the MDR1 efflux transporter, a major efflux transporter at the blood brain barrier (BBB), uses an established method that measures the rate of flux of a compound across polarized cell monolayers. As well appreciated in the art, the data generated from the MDCK-MDR1 permeability assay may be used to predict in vivo absorption of drugs. The bi-directional MDCK-MDR1 permeability assay can identify and quantify levels of active efflux. Screening compounds in both the apical to basolateral direction (A→B) and basolateral to apical direction (B→A) across the cell monolayer provides a ratio of B→A/A→B (efflux ratio; “ER”). Unlike the Caco-2 assay, the MDCK-MDR1 assay is not subject to potential efflux interference by breast cancer resistance protein (BCRP). When a compound has an ER of greater than 2, it suggests that the compound may be subject to active efflux. The data may further be used to predict blood brain barrier (BBB) permeability via calculation from the data of log(permeability-surface area product) (“log PS”), well-understood to be predictive of the in vivo log([brain concentration]/[plasma concentration]).

Table 2 below provides the initial bi-directional MDCK-MDR1 permeability results for exemplary compounds of the present technology.

TABLE 2

| | | MDCK-MDR1 |
|---|---|---|
| | MDCK- | Papp A→B |
| Compound | MDR1 ER | (10⁻⁶ cm/s) |
| 0194A | 2.22 | 6.96 |
| 0207B | 0.532 | 14.4 |
| 0174B | 0.654 | 13.8 |
| 0223B | 37 | 0.685 |
| 0206A | 0.695 | 13.4 |
| 0196A | 0.537 | 13 |
| 0115A | 1.73 | 8.3 |
| 0172A | 0.658 | 11 |
| 0222B | 0.92 | 9.59 |
| 0221B | 1.15 | 8.48 |
| 0195A | 0.699 | 13.9 |
| 0193A | 2.63 | 5.83 |
| 0195B | 0.691 | 16.4 |
| 0211A | 2.67 | 9.25 |
| 0196B | 0.541 | 13.1 |
| 0188A | 0.541 | 11.7 |
| 0189B | 0.538 | 15.6 |
| 0121A | 3.06 | 5.66 |
| 0114-5 | 0.995 | 16.1 |
| 0174A | 0.532 | 14 |
| 0199C | 17.9 | 0.506 |
| 0187B | 1.67 | 8.56 |
| 0114B | 0.55 | 18.6 |
| 0189-6 | 0.59 | 10.6 |
| 0188-5 | 0.522 | 11.7 |
| 0121D | 5.77 | 3.66 |
| 0110B | 30.4 | 0.498 |
| 0120B | 0.612 | 13.3 |
| 0207A | 0.741 | 9.65 |
| 0111B | 33.1 | 0.866 |
| 0120A | 0.655 | 12.9 |
| 0117A | 36.3 | 0.751 |
| 0210A | 6.97 | 2.91 |
| 0111C | 7.76 | 4.46 |
| 0116B | 30.8 | 0.471 |
| 0213B | 3.33 | 4.93 |
| 0210B | 9.91 | 2.27 |
| 0115B | 2.04 | 9.05 |
| 0115C | 4.67 | 4.82 |
| 0218B | 3.98 | 0.335 |
| 0115D | 4.28 | 4.94 |
| 0200B | 12.3 | 1.2 |
| 0215A | 33.3 | 0.373 |
| 0201B | 24.7 | 0.567 |
| 0203B | 21.7 | 0.568 |
| 0202B | 28.5 | 0.77 |
| 0201A; 0201B | 30.1 | 0.558 |
| 0256A | 3.37 | 5.76 |

In vivo CNS Penetration of Exemplary Compounds of the Present Technology

Compounds of the present technology are predicted to show BBB passage according to internal proprietary predictive models. These predictive models were further verified by in vivo experiments. In particular, in vivo unbound brain to plasma ratio (Kpuu) was measured at steady state by comparison of free drug concentrations (protein binding corrected) in brain and plasma after continuous intravenous infusion in Sprague Dawley rats over 24 hours, where a Kpuu of >0.3 represents good brain exposure of the compound.

Table 3 below provides the Kpuu results for exemplary compounds of the present technology. As illustrated in Table 3, compounds of the present technology exhibit in vivo Kpuu values generally accepted to be predictive of clinical brain penetrance.

TABLE 3

| Compound | Kpuu |
|---|---|
| 0114B | 0.921 |
| 0174B | 0.591 |

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims:
A. A compound of Formula I

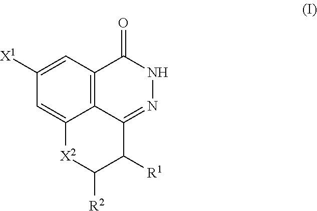

or a pharmaceutically acceptable salt and/or solvate thereof, wherein
   X¹ is H, F, or Cl;
   X² is NH or N—R³
   one of R¹ and R² is H, halo, alkyl, alkenyl, alkynyl, cycloalkyl, or heterocyclyl, and the remaining one of R¹ and R² is aryl, heteroaryl, or non-aromatic heterocyclyl; and
   R³ is alkyl, cycloalkyl, alkylenyl, and non-aromatic heterocyclyl.
B. The compound of Paragraph A, wherein the compound is of Formula IA

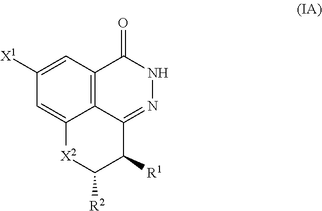

or a pharmaceutically acceptable salt and/or solvate thereof.
C. The compound of Paragraph A, wherein the compound is of Formula IB

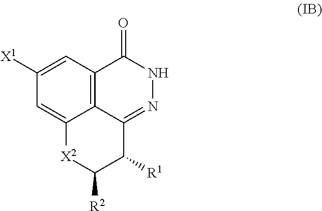

or a pharmaceutically acceptable salt and/or solvate thereof.
D. The compound of any one of Paragraphs A-C, wherein
   R¹ is aryl, heteroaryl, or non-aromatic heterocyclyl; and
   R² is H, halo, alkyl, alkenyl, alkynyl, cycloalkyl, or non-aromatic heterocyclyl.
E. The compound of any one of Paragraphs A-D, wherein
   R¹ is aryl, heteroaryl, or non-aromatic heterocyclyl; and
   R² is H, alkyl, cycloalkyl, or non-aromatic heterocyclyl.
F. The compound of any one of Paragraphs A-E, wherein X¹ is F.
G. The compound of any one of Paragraphs A-F, wherein X² is NH.
H. The compound of any one of Paragraphs A-G, wherein the compound is

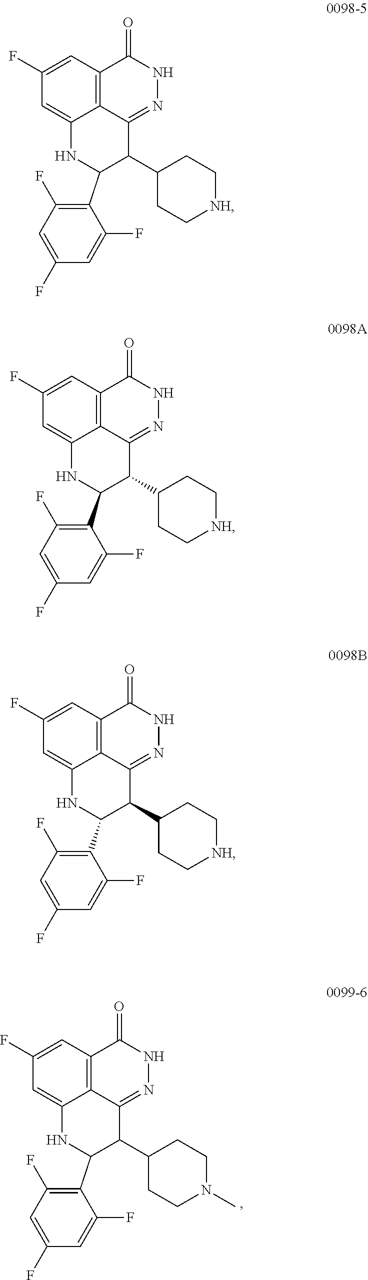 [SMILES: O=c1[nH]nc2c3c(cc(F)cc13)NC(c1c(F)cc(F)cc1F)C2C1CCNCC1 ; O=c1[nH]nc2c3c(cc(F)cc13)N[C@H](c1c(F)cc(F)cc1F)[C@H]2C1CCNCC1 ; O=c1[nH]nc2c3c(cc(F)cc13)N[C@@H](c1c(F)cc(F)cc1F)[C@@H]2C1CCNCC1 ; CN1CCC(C2c3n[nH]c(=O)c4cc(F)cc(c34)NC2c2c(F)cc(F)cc2F)CC1]

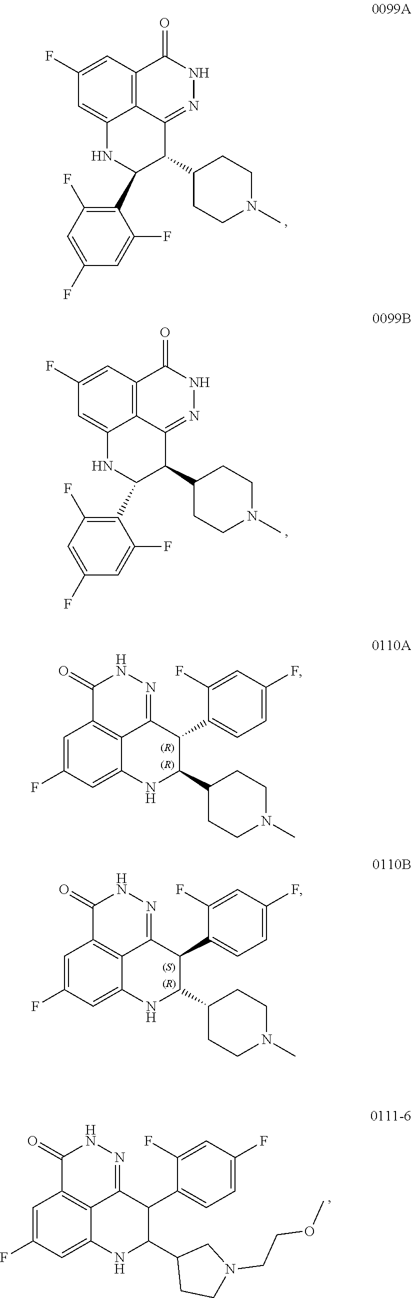

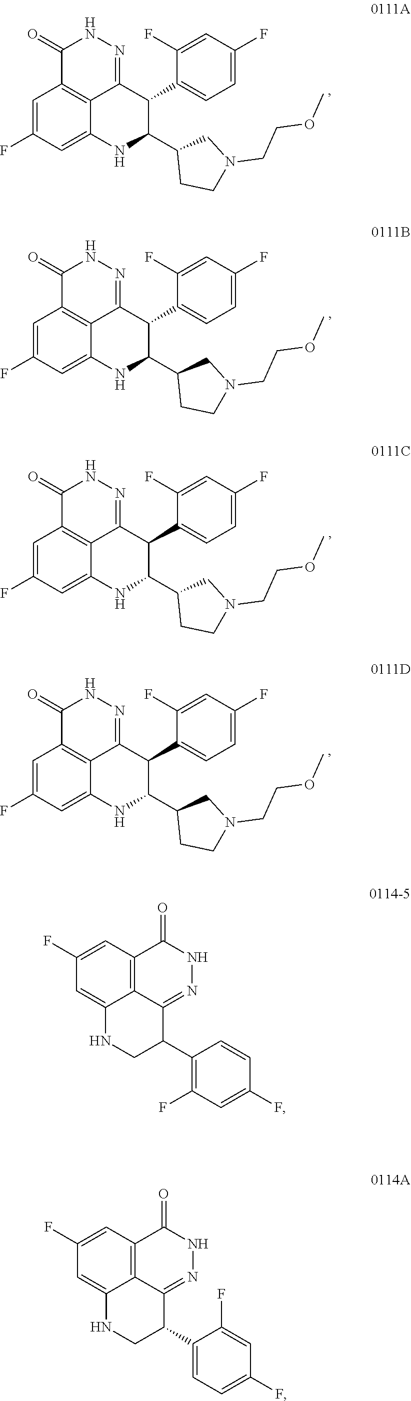 [SMILES: COCCN1CC[C@H]([C@H]2Nc3cc(F)cc4c(=O)[nH]nc(c34)[C@@H]2c2ccc(F)cc2F)C1 ; COCCN1CC[C@@H]([C@H]2Nc3cc(F)cc4c(=O)[nH]nc(c34)[C@@H]2c2ccc(F)cc2F)C1 ; COCCN1CC[C@H]([C@@H]2Nc3cc(F)cc4c(=O)[nH]nc(c34)[C@H]2c2ccc(F)cc2F)C1 ; COCCN1CC[C@@H]([C@@H]2Nc3cc(F)cc4c(=O)[nH]nc(c34)[C@H]2c2ccc(F)cc2F)C1 ; O=c1[nH]nc2c3c(cc(F)cc13)NCC2c1ccc(F)cc1F ; O=c1[nH]nc2c3c(cc(F)cc13)NC[C@H]2c1ccc(F)cc1F]

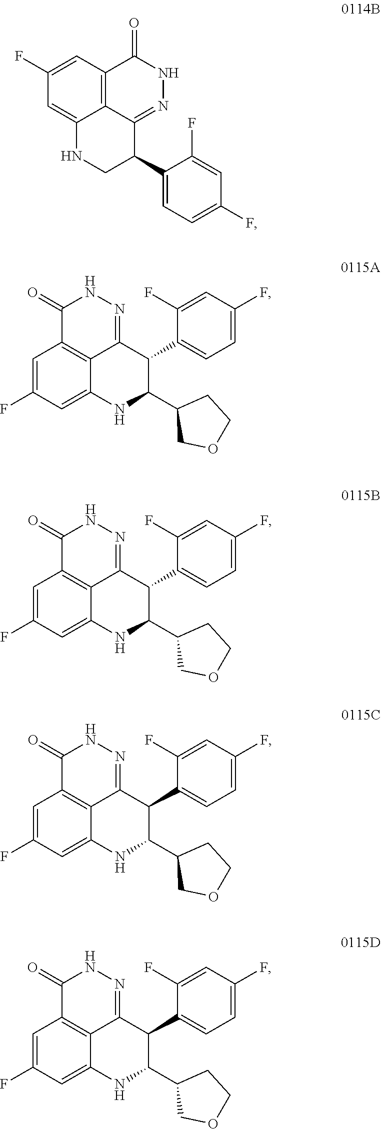 [SMILES: O=c1[nH]nc2c3c(cc(F)cc13)NC[C@@H]2c1ccc(F)cc1F ; O=c1[nH]nc2c3c(cc(F)cc13)N[C@H]([C@H]1CCOC1)[C@H]2c1ccc(F)cc1F ; O=c1[nH]nc2c3c(cc(F)cc13)N[C@H]([C@@H]1CCOC1)[C@H]2c1ccc(F)cc1F ; O=c1[nH]nc2c3c(cc(F)cc13)N[C@@H]([C@H]1CCOC1)[C@@H]2c1ccc(F)cc1F ; O=c1[nH]nc2c3c(cc(F)cc13)N[C@@H]([C@@H]1CCOC1)[C@@H]2c1ccc(F)cc1F]

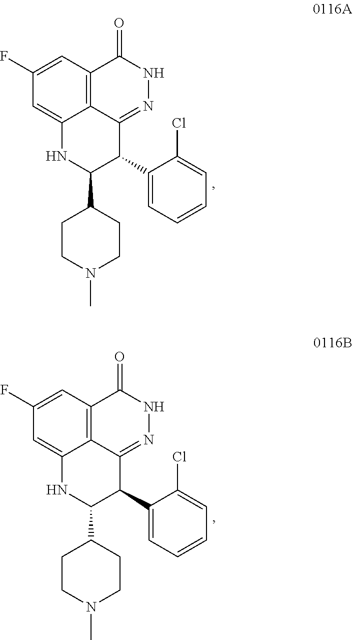 [SMILES: CN1CCC([C@H]2Nc3cc(F)cc4c(=O)[nH]nc(c34)[C@@H]2c2ccccc2Cl)CC1 ; CN1CCC([C@@H]2Nc3cc(F)cc4c(=O)[nH]nc(c34)[C@H]2c2ccccc2Cl)CC1]

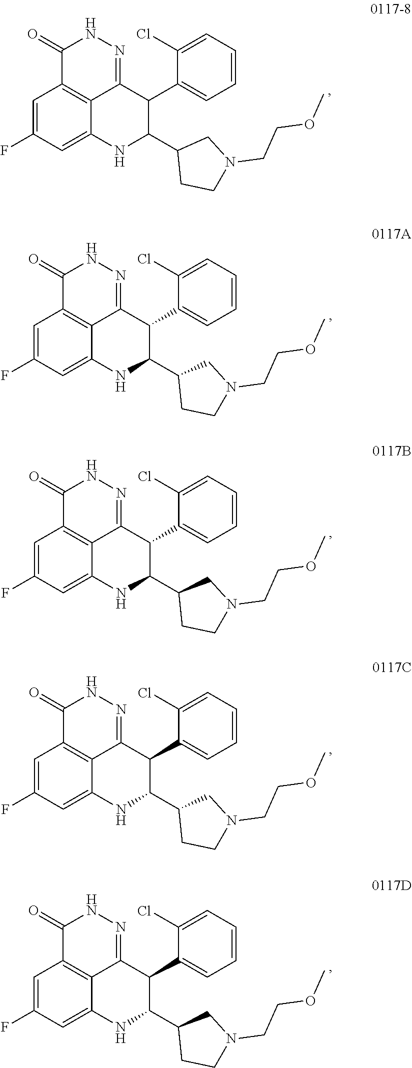 [SMILES: COCCN1CCC(C2Nc3cc(F)cc4c(=O)[nH]nc(c34)C2c2ccccc2Cl)C1 ; COCCN1CC[C@H]([C@H]2Nc3cc(F)cc4c(=O)[nH]nc(c34)[C@@H]2c2ccccc2Cl)C1 ; COCCN1CC[C@@H]([C@H]2Nc3cc(F)cc4c(=O)[nH]nc(c34)[C@@H]2c2ccccc2Cl)C1 ; COCCN1CC[C@H]([C@@H]2Nc3cc(F)cc4c(=O)[nH]nc(c34)[C@H]2c2ccccc2Cl)C1 ; COCCN1CC[C@@H]([C@@H]2Nc3cc(F)cc4c(=O)[nH]nc(c34)[C@H]2c2ccccc2Cl)C1]

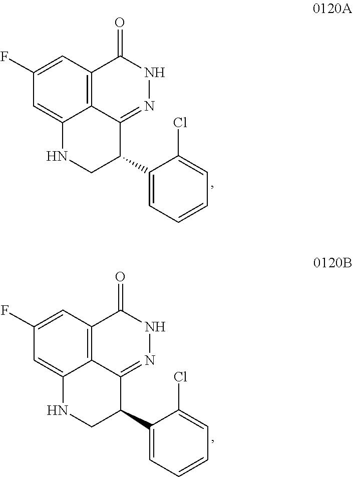 [SMILES: O=c1[nH]nc2c3c(cc(F)cc13)NC[C@H]2c1ccccc1Cl ; O=c1[nH]nc2c3c(cc(F)cc13)NC[C@@H]2c1ccccc1Cl]

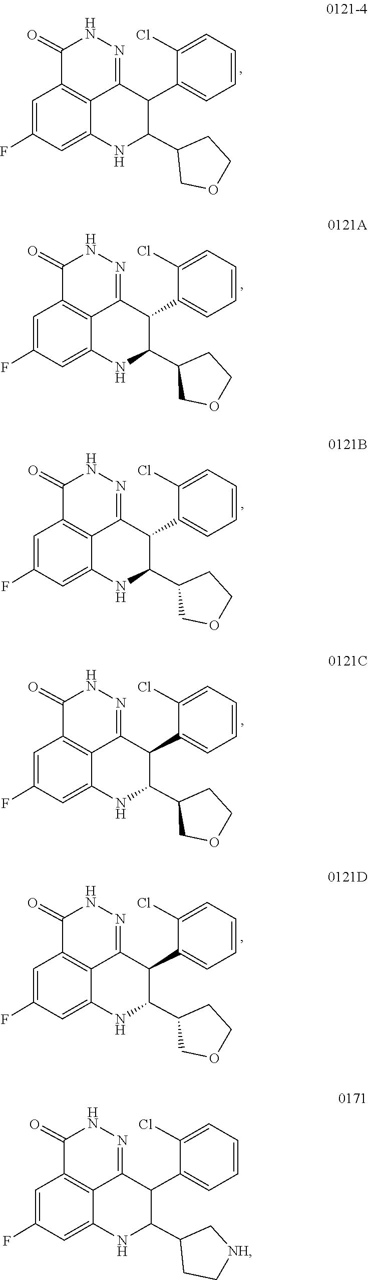 [SMILES: O=c1[nH]nc2c3c(cc(F)cc13)NC(C1CCOC1)C2c1ccccc1Cl ; O=c1[nH]nc2c3c(cc(F)cc13)N[C@H]([C@H]1CCOC1)[C@H]2c1ccccc1Cl ; O=c1[nH]nc2c3c(cc(F)cc13)N[C@H]([C@@H]1CCOC1)[C@H]2c1ccccc1Cl ; O=c1[nH]nc2c3c(cc(F)cc13)N[C@@H]([C@H]1CCOC1)[C@@H]2c1ccccc1Cl ; O=c1[nH]nc2c3c(cc(F)cc13)N[C@@H]([C@@H]1CCOC1)[C@@H]2c1ccccc1Cl ; O=c1[nH]nc2c3c(cc(F)cc13)NC(C1CCNC1)C2c1ccccc1Cl]

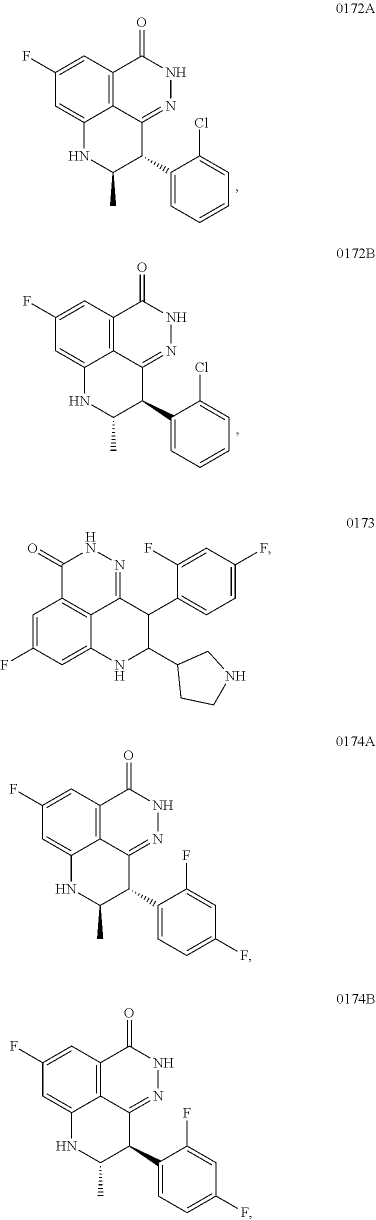 [SMILES: C[C@H]1Nc2cc(F)cc3c(=O)[nH]nc(c23)[C@@H]1c1ccccc1Cl ; C[C@@H]1Nc2cc(F)cc3c(=O)[nH]nc(c23)[C@H]1c1ccccc1Cl ; O=c1[nH]nc2c3c(cc(F)cc13)NC(C1CCNC1)C2c1ccc(F)cc1F ; C[C@H]1Nc2cc(F)cc3c(=O)[nH]nc(c23)[C@@H]1c1ccc(F)cc1F ; C[C@@H]1Nc2cc(F)cc3c(=O)[nH]nc(c23)[C@H]1c1ccc(F)cc1F]

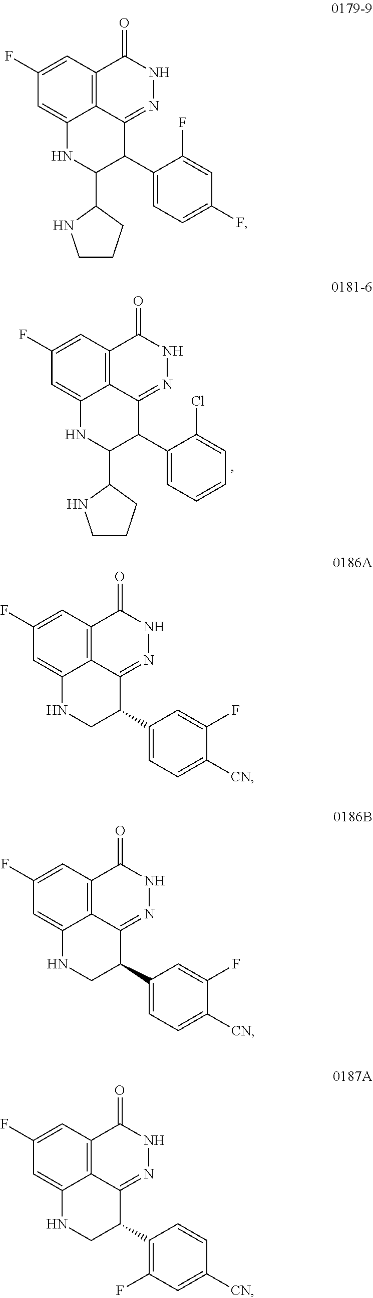

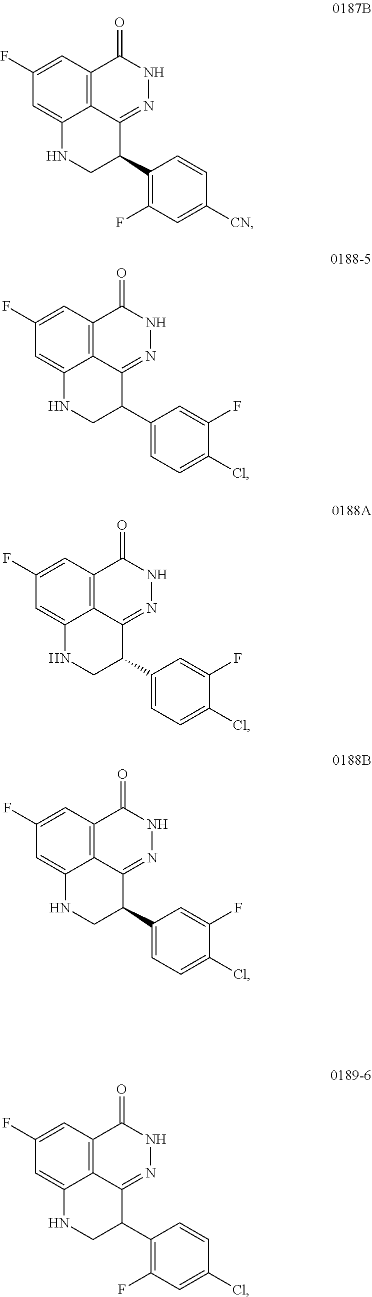 [SMILES: N#Cc1ccc([C@H]2CNc3cc(F)cc4c(=O)[nH]nc2c34)c(F)c1 ; O=c1[nH]nc2c3c(cc(F)cc13)NCC2c1ccc(Cl)c(F)c1 ; O=c1[nH]nc2c3c(cc(F)cc13)NC[C@H]2c1ccc(Cl)c(F)c1 ; O=c1[nH]nc2c3c(cc(F)cc13)NC[C@@H]2c1ccc(Cl)c(F)c1 ; O=c1[nH]nc2c3c(cc(F)cc13)NCC2c1ccc(Cl)cc1F]

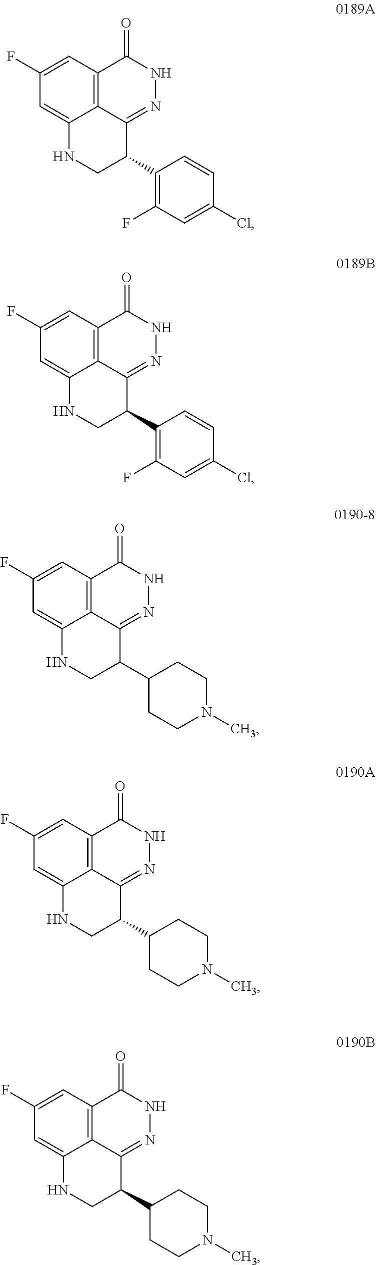 [SMILES: O=c1[nH]nc2c3c(cc(F)cc13)NC[C@H]2c1ccc(Cl)cc1F ; O=c1[nH]nc2c3c(cc(F)cc13)NC[C@@H]2c1ccc(Cl)cc1F ; CN1CCC(C2CNc3cc(F)cc4c(=O)[nH]nc2c34)CC1 ; CN1CCC([C@@H]2CNc3cc(F)cc4c(=O)[nH]nc2c34)CC1 ; CN1CCC([C@H]2CNc3cc(F)cc4c(=O)[nH]nc2c34)CC1]

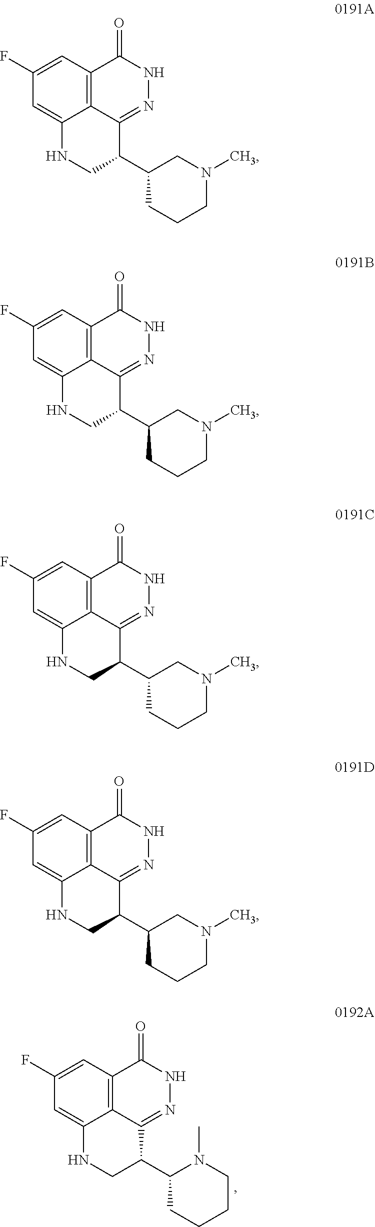 [SMILES: CN1CCC[C@H]([C@@H]2CNc3cc(F)cc4c(=O)[nH]nc2c34)C1 ; CN1CCC[C@@H]([C@@H]2CNc3cc(F)cc4c(=O)[nH]nc2c34)C1 ; CN1CCC[C@H]([C@H]2CNc3cc(F)cc4c(=O)[nH]nc2c34)C1 ; CN1CCC[C@@H]([C@H]2CNc3cc(F)cc4c(=O)[nH]nc2c34)C1 ; CN1CCCC[C@@H]1[C@@H]1CNc2cc(F)cc3c(=O)[nH]nc1c23]

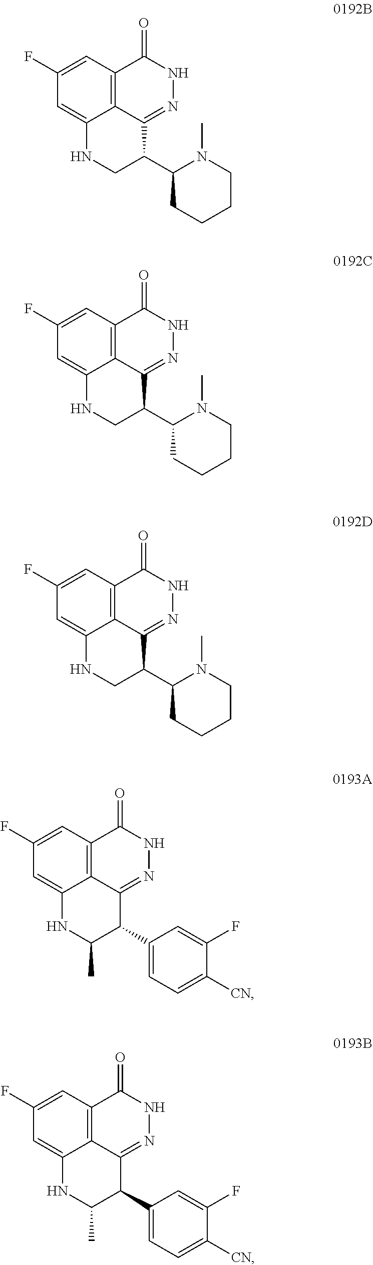 [SMILES: CN1CCCC[C@H]1[C@@H]1CNc2cc(F)cc3c(=O)[nH]nc1c23 ; CN1CCCC[C@@H]1[C@H]1CNc2cc(F)cc3c(=O)[nH]nc1c23 ; CN1CCCC[C@H]1[C@H]1CNc2cc(F)cc3c(=O)[nH]nc1c23 ; C[C@H]1Nc2cc(F)cc3c(=O)[nH]nc(c23)[C@@H]1c1ccc(C#N)c(F)c1 ; C[C@@H]1Nc2cc(F)cc3c(=O)[nH]nc(c23)[C@H]1c1ccc(C#N)c(F)c1]

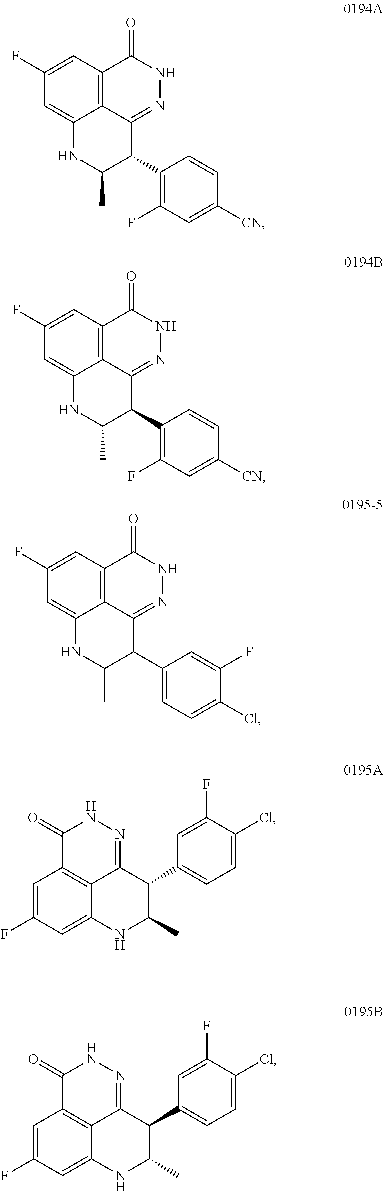

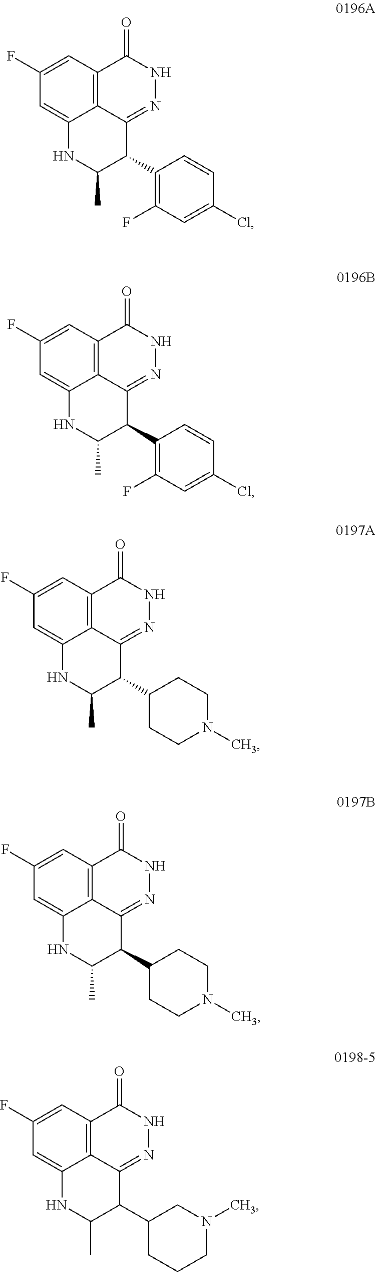 [SMILES: C[C@H]1Nc2cc(F)cc3c(=O)[nH]nc(c23)[C@@H]1c1ccc(Cl)cc1F ; C[C@@H]1Nc2cc(F)cc3c(=O)[nH]nc(c23)[C@H]1c1ccc(Cl)cc1F ; C[C@H]1Nc2cc(F)cc3c(=O)[nH]nc(c23)[C@@H]1C1CCN(C)CC1 ; C[C@@H]1Nc2cc(F)cc3c(=O)[nH]nc(c23)[C@H]1C1CCN(C)CC1 ; CC1Nc2cc(F)cc3c(=O)[nH]nc(c23)C1C1CCCN(C)C1]

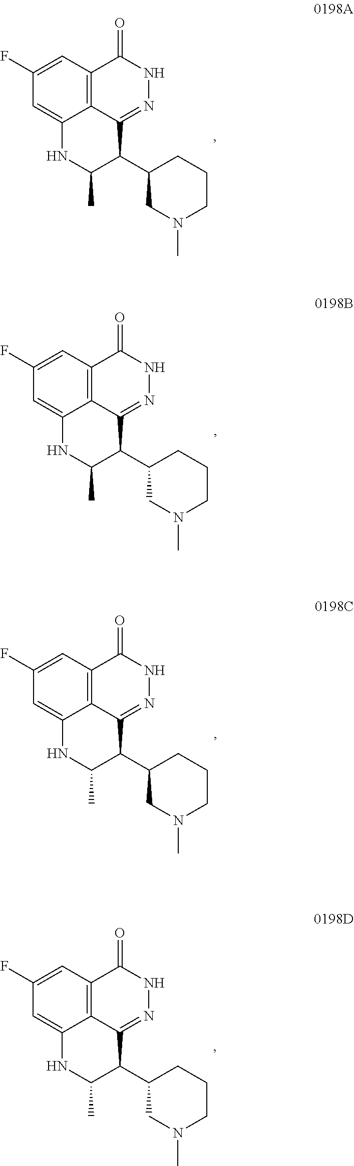 [SMILES: C[C@H]1Nc2cc(F)cc3c(=O)[nH]nc(c23)[C@H]1[C@H]1CCCN(C)C1 ; C[C@H]1Nc2cc(F)cc3c(=O)[nH]nc(c23)[C@H]1[C@@H]1CCCN(C)C1 ; C[C@@H]1Nc2cc(F)cc3c(=O)[nH]nc(c23)[C@H]1[C@H]1CCCN(C)C1 ; C[C@@H]1Nc2cc(F)cc3c(=O)[nH]nc(c23)[C@H]1[C@@H]1CCCN(C)C1]

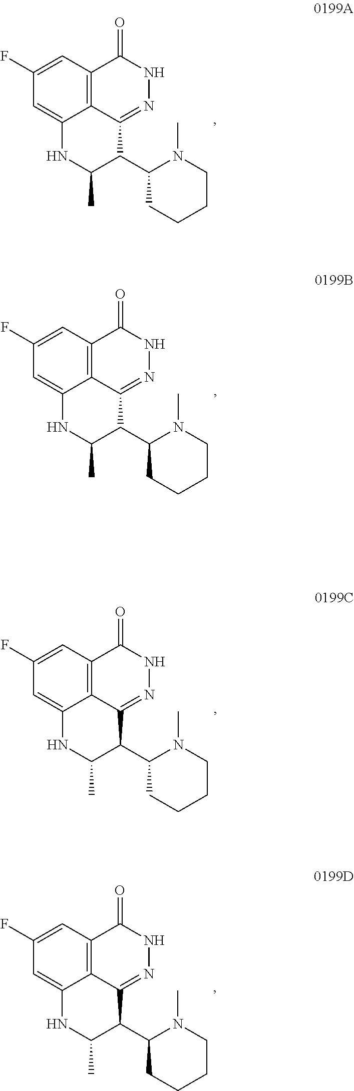

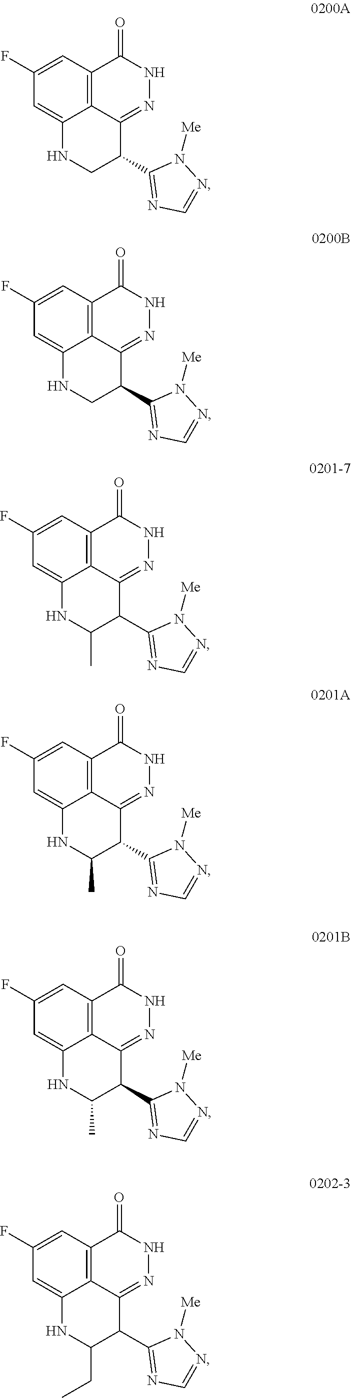 [SMILES: Cn1ncnc1[C@@H]1CNc2cc(F)cc3c(=O)[nH]nc1c23 ; Cn1ncnc1[C@H]1CNc2cc(F)cc3c(=O)[nH]nc1c23 ; CC1Nc2cc(F)cc3c(=O)[nH]nc(c23)C1c1ncnn1C ; C[C@H]1Nc2cc(F)cc3c(=O)[nH]nc(c23)[C@@H]1c1ncnn1C ; C[C@@H]1Nc2cc(F)cc3c(=O)[nH]nc(c23)[C@H]1c1ncnn1C ; CCC1Nc2cc(F)cc3c(=O)[nH]nc(c23)C1c1ncnn1C]

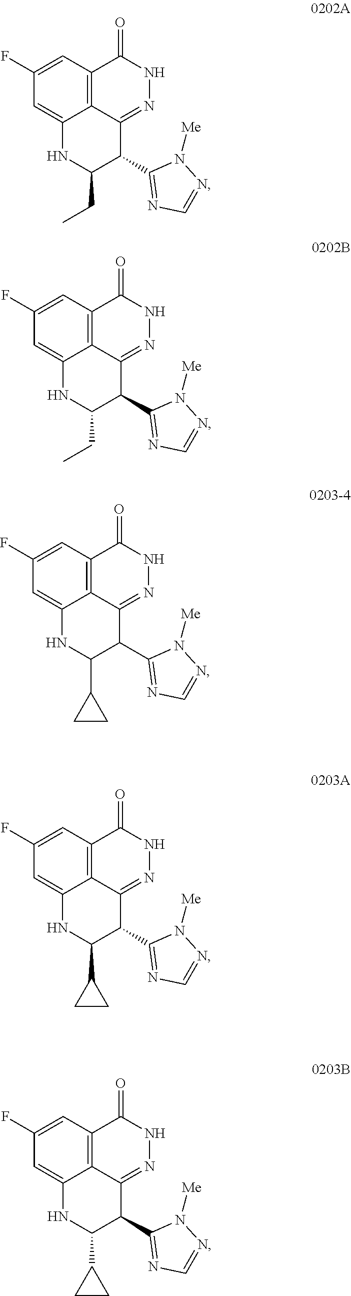 [SMILES: CC[C@H]1Nc2cc(F)cc3c(=O)[nH]nc(c23)[C@@H]1c1ncnn1C ; CC[C@@H]1Nc2cc(F)cc3c(=O)[nH]nc(c23)[C@H]1c1ncnn1C ; Cn1ncnc1C1c2n[nH]c(=O)c3cc(F)cc(c23)NC1C1CC1 ; Cn1ncnc1[C@H]1c2n[nH]c(=O)c3cc(F)cc(c23)N[C@@H]1C1CC1 ; Cn1ncnc1[C@@H]1c2n[nH]c(=O)c3cc(F)cc(c23)N[C@H]1C1CC1]

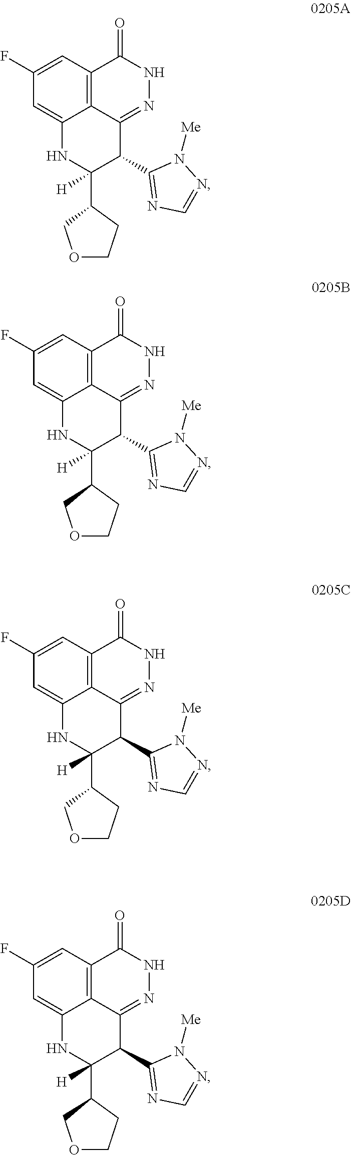

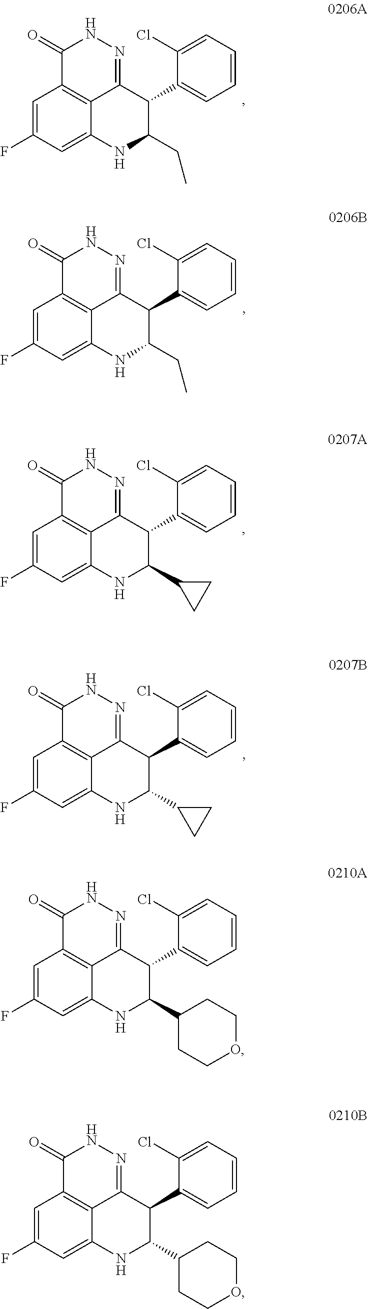 [SMILES: CC[C@H]1Nc2cc(F)cc3c(=O)[nH]nc(c23)[C@@H]1c1ccccc1Cl ; CC[C@@H]1Nc2cc(F)cc3c(=O)[nH]nc(c23)[C@H]1c1ccccc1Cl ; O=c1[nH]nc2c3c(cc(F)cc13)N[C@H](C1CC1)[C@H]2c1ccccc1Cl ; O=c1[nH]nc2c3c(cc(F)cc13)N[C@@H](C1CC1)[C@@H]2c1ccccc1Cl ; O=c1[nH]nc2c3c(cc(F)cc13)N[C@H](C1CCOCC1)[C@H]2c1ccccc1Cl ; O=c1[nH]nc2c3c(cc(F)cc13)N[C@@H](C1CCOCC1)[C@@H]2c1ccccc1Cl]

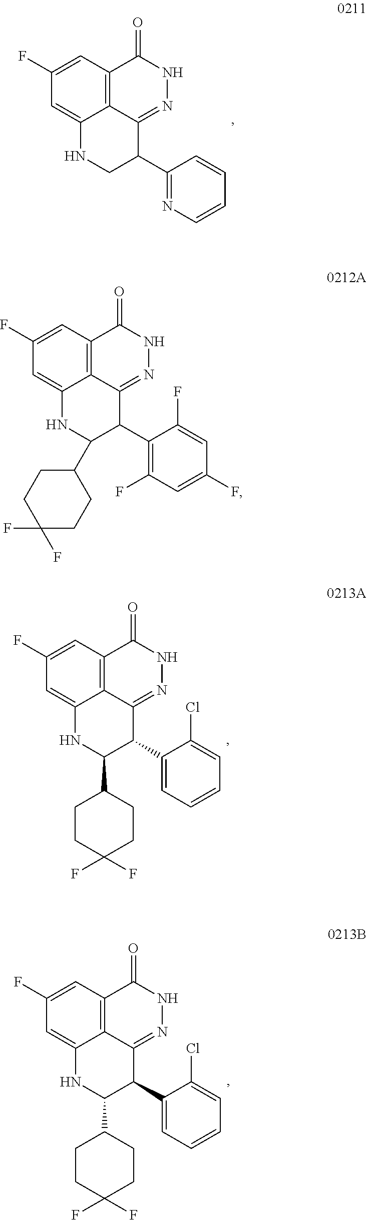 [SMILES: O=c1[nH]nc2c3c(cc(F)cc13)NCC2c1ccccn1 ; O=c1[nH]nc2c3c(cc(F)cc13)NC(C1CCC(F)(F)CC1)C2c1c(F)cc(F)cc1F ; O=c1[nH]nc2c3c(cc(F)cc13)N[C@H](C1CCC(F)(F)CC1)[C@H]2c1ccccc1Cl ; O=c1[nH]nc2c3c(cc(F)cc13)N[C@@H](C1CCC(F)(F)CC1)[C@@H]2c1ccccc1Cl]

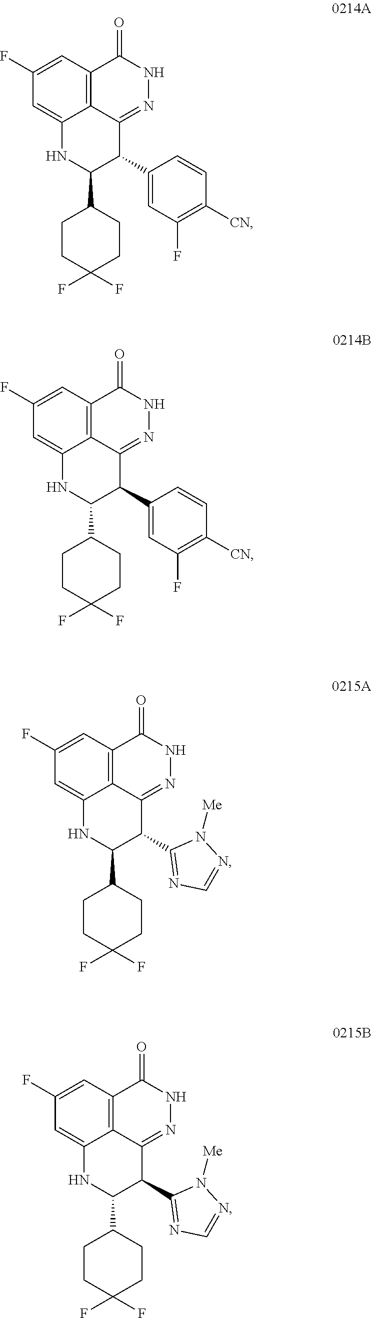 [SMILES: N#Cc1ccc([C@H]2c3n[nH]c(=O)c4cc(F)cc(c34)N[C@@H]2C2CCC(F)(F)CC2)cc1F ; N#Cc1ccc([C@@H]2c3n[nH]c(=O)c4cc(F)cc(c34)N[C@H]2C2CCC(F)(F)CC2)cc1F ; Cn1ncnc1[C@H]1c2n[nH]c(=O)c3cc(F)cc(c23)N[C@@H]1C1CCC(F)(F)CC1 ; Cn1ncnc1[C@@H]1c2n[nH]c(=O)c3cc(F)cc(c23)N[C@H]1C1CCC(F)(F)CC1]

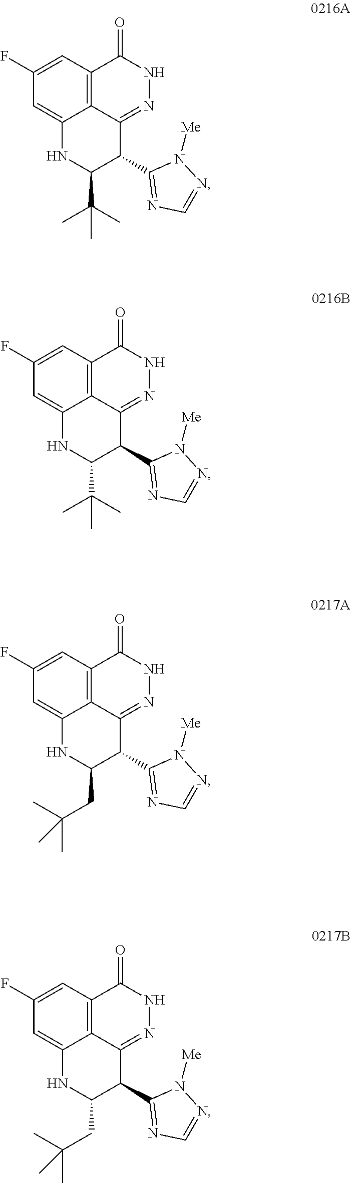 [SMILES: Cn1ncnc1[C@H]1c2n[nH]c(=O)c3cc(F)cc(c23)N[C@@H]1C(C)(C)C ; Cn1ncnc1[C@@H]1c2n[nH]c(=O)c3cc(F)cc(c23)N[C@H]1C(C)(C)C ; Cn1ncnc1[C@H]1c2n[nH]c(=O)c3cc(F)cc(c23)N[C@@H]1CC(C)(C)C ; Cn1ncnc1[C@@H]1c2n[nH]c(=O)c3cc(F)cc(c23)N[C@H]1CC(C)(C)C]

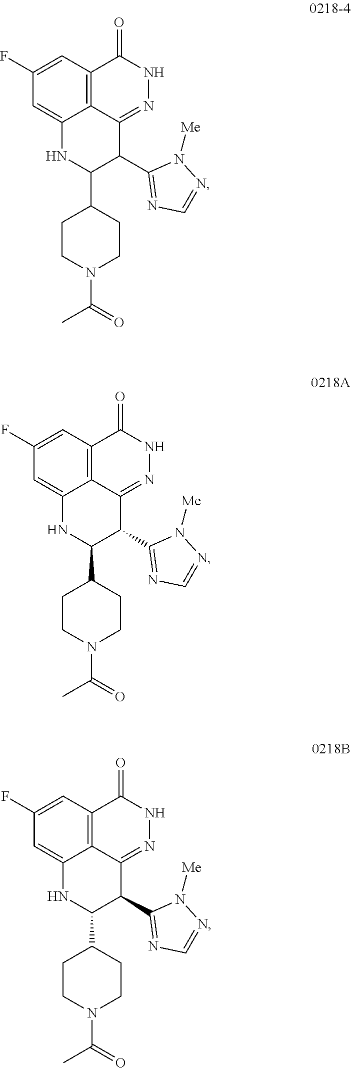 [SMILES: CC(=O)N1CCC(C2Nc3cc(F)cc4c(=O)[nH]nc(c34)C2c2ncnn2C)CC1 ; CC(=O)N1CCC([C@H]2Nc3cc(F)cc4c(=O)[nH]nc(c34)[C@@H]2c2ncnn2C)CC1 ; CC(=O)N1CCC([C@@H]2Nc3cc(F)cc4c(=O)[nH]nc(c34)[C@H]2c2ncnn2C)CC1]

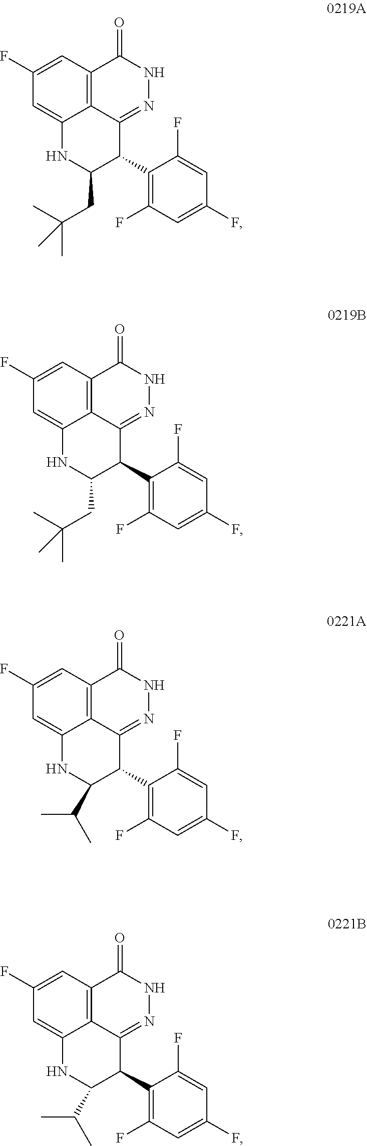 [SMILES: CC(C)(C)C[C@H]1Nc2cc(F)cc3c(=O)[nH]nc(c23)[C@@H]1c1c(F)cc(F)cc1F ; CC(C)(C)C[C@@H]1Nc2cc(F)cc3c(=O)[nH]nc(c23)[C@H]1c1c(F)cc(F)cc1F ; CC(C)[C@H]1Nc2cc(F)cc3c(=O)[nH]nc(c23)[C@@H]1c1c(F)cc(F)cc1F ; CC(C)[C@@H]1Nc2cc(F)cc3c(=O)[nH]nc(c23)[C@H]1c1c(F)cc(F)cc1F]

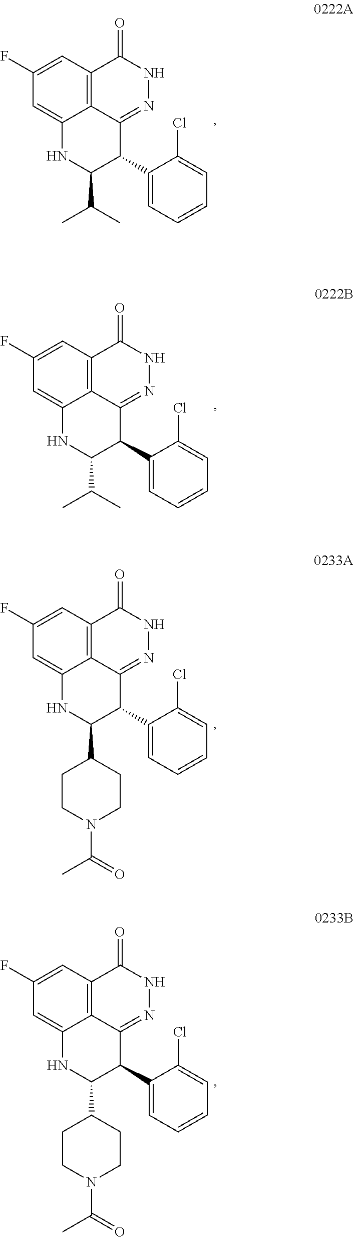 [SMILES: CC(C)[C@H]1Nc2cc(F)cc3c(=O)[nH]nc(c23)[C@@H]1c1ccccc1Cl ; CC(C)[C@@H]1Nc2cc(F)cc3c(=O)[nH]nc(c23)[C@H]1c1ccccc1Cl ; CC(=O)N1CCC([C@H]2Nc3cc(F)cc4c(=O)[nH]nc(c34)[C@@H]2c2ccccc2Cl)CC1 ; CC(=O)N1CCC([C@@H]2Nc3cc(F)cc4c(=O)[nH]nc(c34)[C@H]2c2ccccc2Cl)CC1]

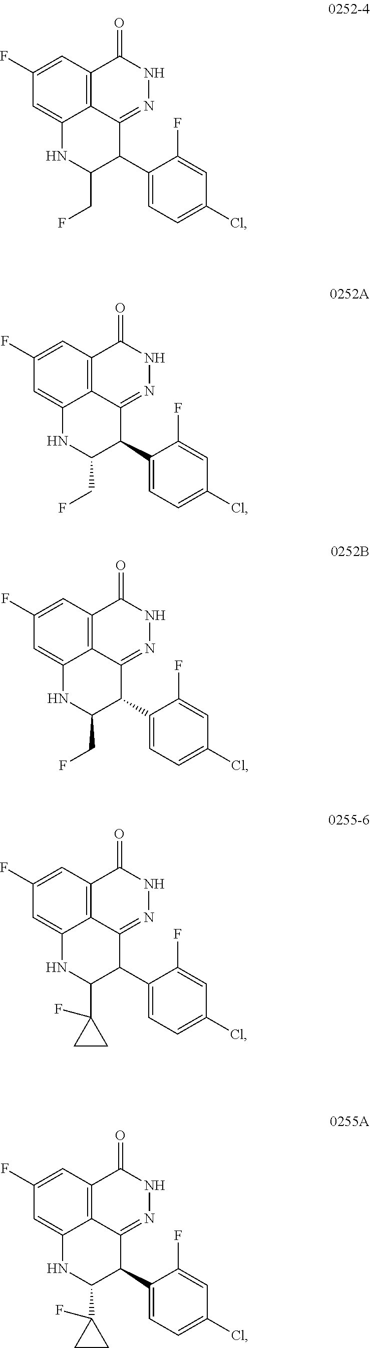 [SMILES: O=c1[nH]nc2c3c(cc(F)cc13)NC(CF)C2c1ccc(Cl)cc1F ; O=c1[nH]nc2c3c(cc(F)cc13)N[C@@H](CF)[C@@H]2c1ccc(Cl)cc1F ; O=c1[nH]nc2c3c(cc(F)cc13)N[C@H](CF)[C@H]2c1ccc(Cl)cc1F ; O=c1[nH]nc2c3c(cc(F)cc13)NC(C1(F)CC1)C2c1ccc(Cl)cc1F ; O=c1[nH]nc2c3c(cc(F)cc13)N[C@@H](C1(F)CC1)[C@@H]2c1ccc(Cl)cc1F]

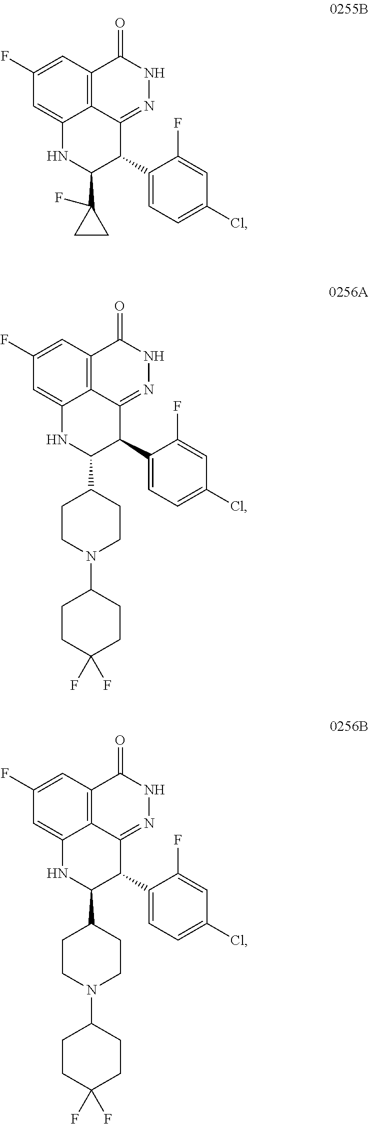 [SMILES: O=c1[nH]nc2c3c(cc(F)cc13)N[C@H](C1(F)CC1)[C@H]2c1ccc(Cl)cc1F ; O=c1[nH]nc2c3c(cc(F)cc13)N[C@@H](C1CCN(C3CCC(F)(F)CC3)CC1)[C@@H]2c1ccc(Cl)cc1F ; O=c1[nH]nc2c3c(cc(F)cc13)N[C@H](C1CCN(C3CCC(F)(F)CC3)CC1)[C@H]2c1ccc(Cl)cc1F]

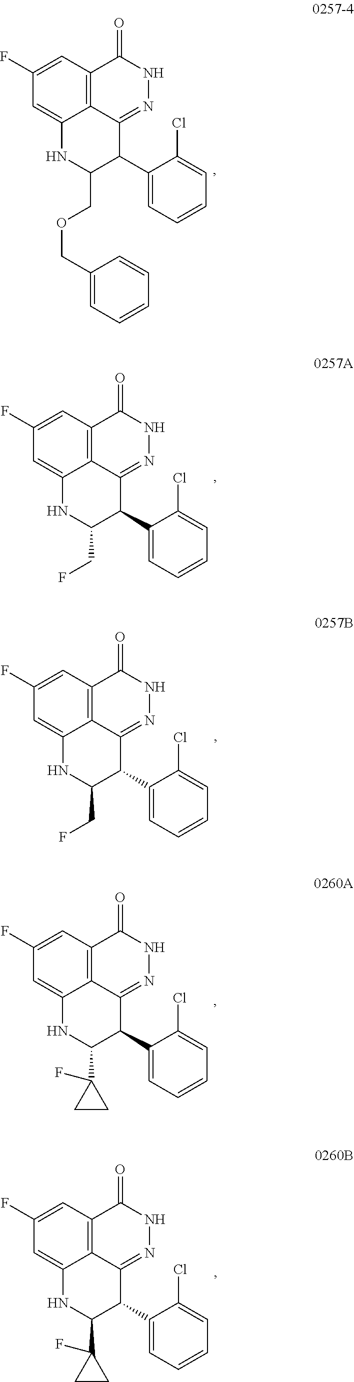 [SMILES: O=c1[nH]nc2c3c(cc(F)cc13)NC(COCc1ccccc1)C2c1ccccc1Cl ; O=c1[nH]nc2c3c(cc(F)cc13)N[C@@H](CF)[C@@H]2c1ccccc1Cl ; O=c1[nH]nc2c3c(cc(F)cc13)N[C@H](CF)[C@H]2c1ccccc1Cl ; O=c1[nH]nc2c3c(cc(F)cc13)N[C@@H](C1(F)CC1)[C@@H]2c1ccccc1Cl ; O=c1[nH]nc2c3c(cc(F)cc13)N[C@H](C1(F)CC1)[C@H]2c1ccccc1Cl]

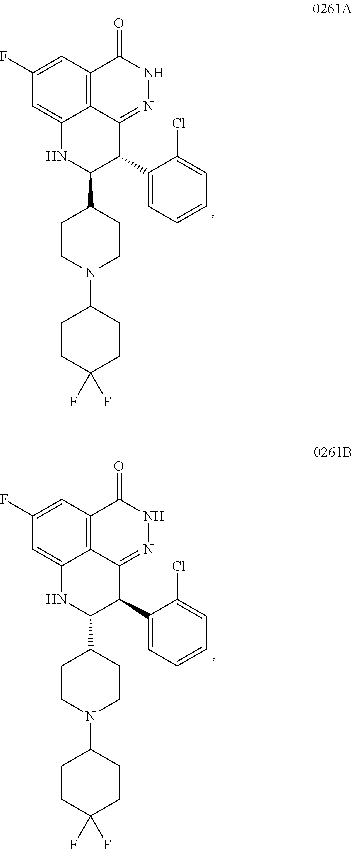 [SMILES: O=c1[nH]nc2c3c(cc(F)cc13)N[C@H](C1CCN(C3CCC(F)(F)CC3)CC1)[C@H]2c1ccccc1Cl ; O=c1[nH]nc2c3c(cc(F)cc13)N[C@@H](C1CCN(C3CCC(F)(F)CC3)CC1)[C@@H]2c1ccccc1Cl]

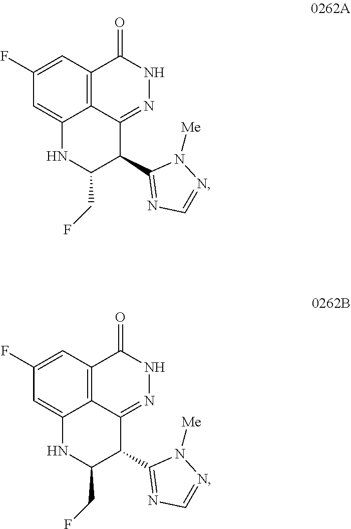 [SMILES: Cn1ncnc1[C@@H]1c2n[nH]c(=O)c3cc(F)cc(c23)N[C@H]1CF ; Cn1ncnc1[C@H]1c2n[nH]c(=O)c3cc(F)cc(c23)N[C@@H]1CF]

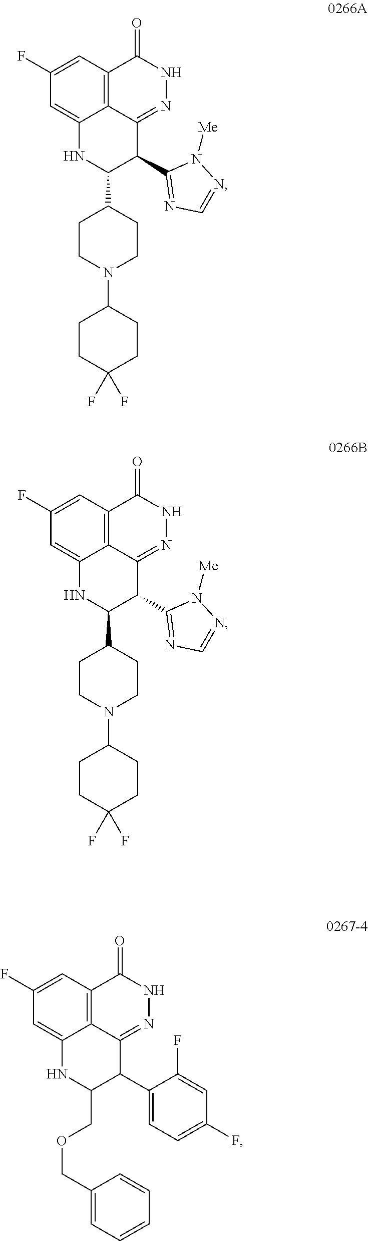 [SMILES: Cn1ncnc1[C@@H]1c2n[nH]c(=O)c3cc(F)cc(c23)N[C@H]1C1CCN(C2CCC(F)(F)CC2)CC1 ; Cn1ncnc1[C@H]1c2n[nH]c(=O)c3cc(F)cc(c23)N[C@@H]1C1CCN(C2CCC(F)(F)CC2)CC1 ; O=c1[nH]nc2c3c(cc(F)cc13)NC(COCc1ccccc1)C2c1ccc(F)cc1F]

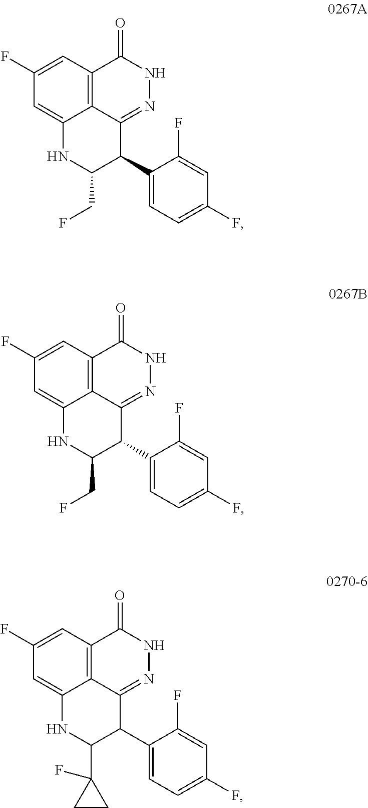 [SMILES: O=c1[nH]nc2c3c(cc(F)cc13)N[C@@H](CF)[C@@H]2c1ccc(F)cc1F ; O=c1[nH]nc2c3c(cc(F)cc13)N[C@H](CF)[C@H]2c1ccc(F)cc1F ; O=c1[nH]nc2c3c(cc(F)cc13)NC(C1(F)CC1)C2c1ccc(F)cc1F]

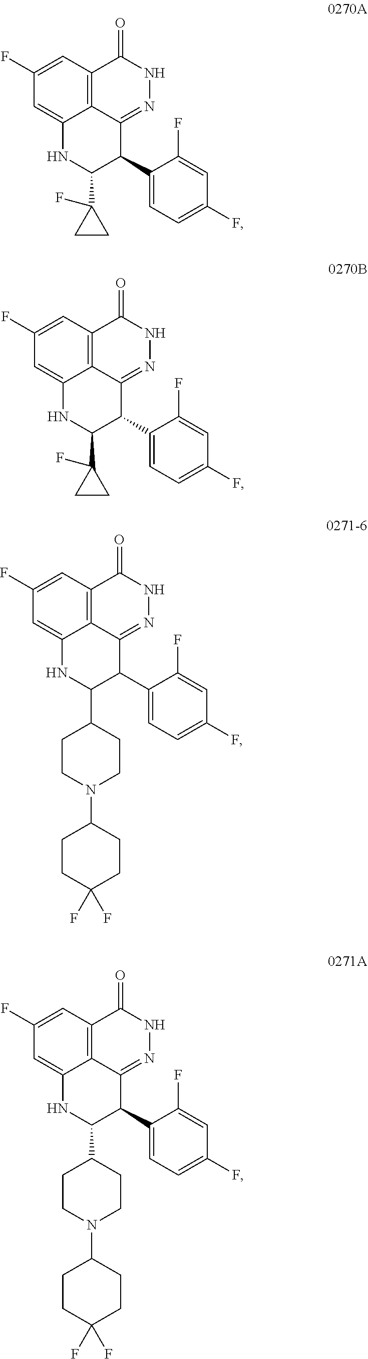

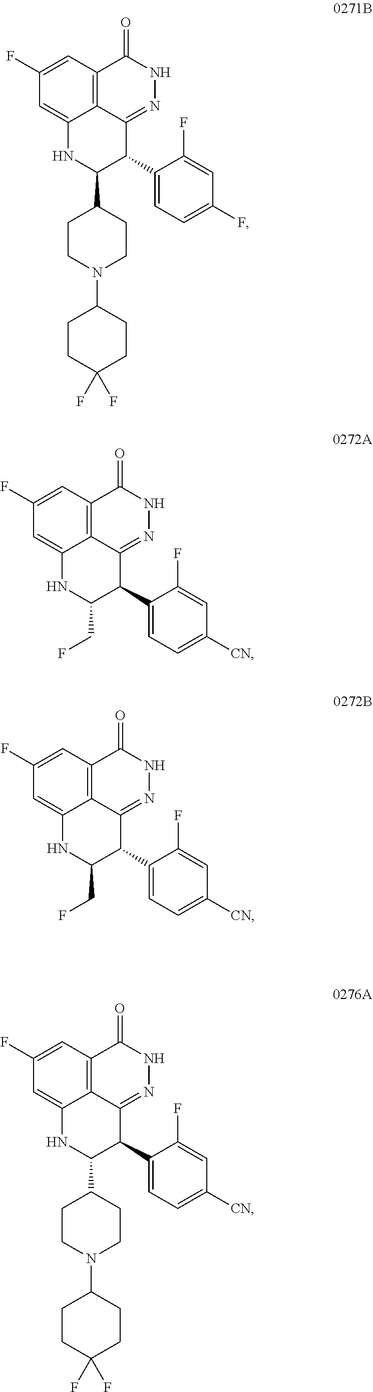 [SMILES: O=c1[nH]nc2c3c(cc(F)cc13)N[C@H](C1CCN(C3CCC(F)(F)CC3)CC1)[C@H]2c1ccc(F)cc1F ; N#Cc1ccc([C@@H]2c3n[nH]c(=O)c4cc(F)cc(c34)N[C@H]2CF)c(F)c1 ; N#Cc1ccc([C@H]2c3n[nH]c(=O)c4cc(F)cc(c34)N[C@@H]2CF)c(F)c1 ; N#Cc1ccc([C@@H]2c3n[nH]c(=O)c4cc(F)cc(c34)N[C@H]2C2CCN(C3CCC(F)(F)CC3)CC2)c(F)c1]

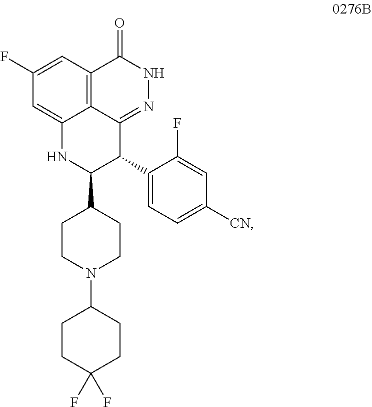 [SMILES: N#Cc1ccc([C@H]2c3n[nH]c(=O)c4cc(F)cc(c34)N[C@@H]2C2CCN(C3CCC(F)(F)CC3)CC2)c(F)c1]

or a pharmaceutically acceptable salt and/or solvate of any one thereof.
I. A composition comprising a compound of any one of Paragraphs A-H and a pharmaceutically acceptable carrier.
J. A pharmaceutical composition comprising a pharmaceutically acceptable carrier and an effective amount of a compound of any one of Paragraphs A-H, wherein the effective amount of the compound is effective to treat a cancer.
K. A pharmaceutical composition comprising a pharmaceutically acceptable carrier and a compound of any one of Paragraphs A-H, wherein the compound is present in an amount effective to treat the cancer when combined with an second cancer therapy.
L. A method of treating a subject suffering from a B-cell malignancy, the method comprising administering to the subject an effective amount of a compound of any one of Paragraphs A-H and an effective amount of a second cancer therapy.
M. A medicament for treating a cancer in a subject, the medicament comprising a compound of any one of Paragraphs A-H.
N. The medicament of Paragraph M, wherein the medicament further comprises a pharmaceutically acceptable carrier.
O. The medicament of Paragraph M or Paragraph N, wherein the medicament comprises an effective amount of the compound for treating the cancer when combined with a second cancer therapy.

Other embodiments are set forth in the following claims, along with the full scope of equivalents to which such claims are entitled.

1. A compound of Formula I 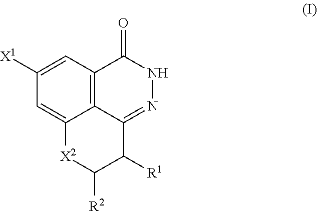
or a pharmaceutically acceptable salt and/or solvate thereof, wherein
X¹ is H, F, or Cl;
X² is NH or N—R³;
one of R¹ and R² is H, halo, alkyl, alkenyl, alkynyl, cycloalkyl, or heterocyclyl, and the remaining one of R¹ and R² is aryl, heteroaryl, or non-aromatic heterocyclyl; and
R³ is alkyl, cycloalkyl, alkylenyl, and non-aromatic heterocyclyl.

2. The compound of claim 1, wherein
R¹ is aryl, heteroaryl, or non-aromatic heterocyclyl; and
R² is H, halo, alkyl, alkenyl, alkynyl, cycloalkyl, or non-aromatic heterocyclyl.

3. The compound of claim 1, wherein
R¹ is aryl, heteroaryl, or non-aromatic heterocyclyl; and
R² is H, alkyl, cycloalkyl, or non-aromatic heterocyclyl.

4. The compound of claim 1, wherein X¹ is F.

5. The compound of claim 1, wherein X² is NH.

6. The compound of claim 1, wherein the compound is of Formula IA 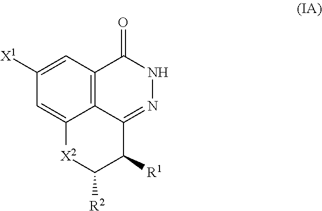
or a pharmaceutically acceptable salt and/or solvate thereof.

7. The compound of claim 6, wherein
R¹ is aryl, heteroaryl, or non-aromatic heterocyclyl; and
R² is H, halo, alkyl, alkenyl, alkynyl, cycloalkyl, or non-aromatic heterocyclyl.

8. The compound of claim 7, wherein
R¹ is aryl, heteroaryl, or non-aromatic heterocyclyl; and
R² is H, alkyl, cycloalkyl, or non-aromatic heterocyclyl.

9. The compound of claim 8, wherein X¹ is F.

10. The compound of claim 9, wherein X² is NH.

11. The compound of claim 1, wherein the compound is of Formula IB 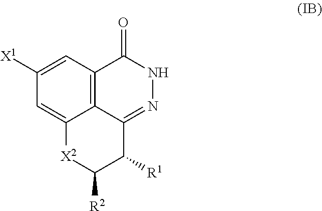
or a pharmaceutically acceptable salt and/or solvate thereof.

12. The compound of claim 11, wherein
R¹ is aryl, heteroaryl, or non-aromatic heterocyclyl; and
R² is H, halo, alkyl, alkenyl, alkynyl, cycloalkyl, or non-aromatic heterocyclyl.

13. The compound of claim 12, wherein
R¹ is aryl, heteroaryl, or non-aromatic heterocyclyl; and
R² is H, alkyl, cycloalkyl, or non-aromatic heterocyclyl.

14. The compound of claim 13, wherein X¹ is F.

15. The compound of claim 14, wherein X² is NH.

16. A composition comprising a compound of claim 1 and a pharmaceutically acceptable carrier.

17. A pharmaceutical composition comprising a pharmaceutically acceptable carrier and an effective amount of a compound of claim 1, wherein the effective amount of the compound is effective to treat a cancer.

18. A pharmaceutical composition comprising a pharmaceutically acceptable carrier and a compound of claim 1, wherein the compound is present in an amount effective to treat the cancer when combined with a second cancer therapy.

19. A method of treating a subject suffering from a B-cell malignancy, the method comprising administering to the subject an effective amount of a compound of claim 1 and an effective amount of a second cancer therapy.

20.-23. (canceled)

24. The compound of claim 1, wherein the compound is 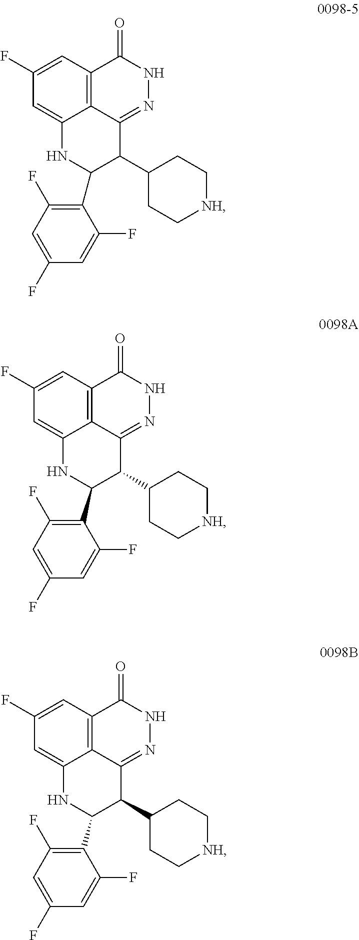 [SMILES: O=c1[nH]nc2c3c(cc(F)cc13)NC(c1c(F)cc(F)cc1F)C2C1CCNCC1 ; O=c1[nH]nc2c3c(cc(F)cc13)N[C@H](c1c(F)cc(F)cc1F)[C@H]2C1CCNCC1 ; O=c1[nH]nc2c3c(cc(F)cc13)N[C@@H](c1c(F)cc(F)cc1F)[C@@H]2C1CCNCC1] 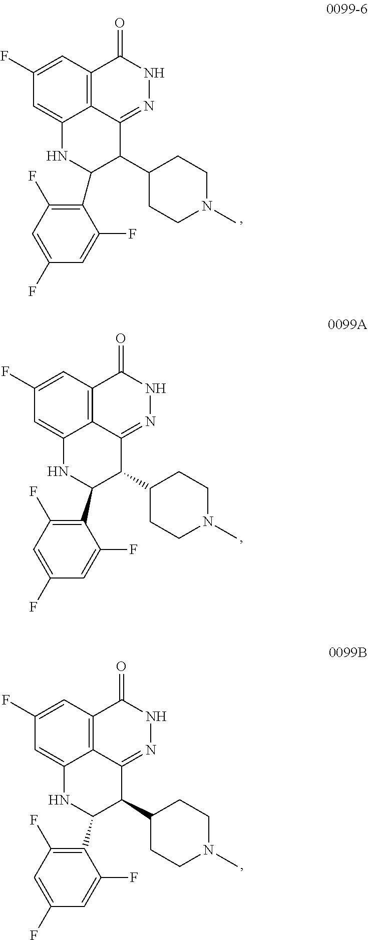 [SMILES: CN1CCC(C2c3n[nH]c(=O)c4cc(F)cc(c34)NC2c2c(F)cc(F)cc2F)CC1 ; CN1CCC([C@H]2c3n[nH]c(=O)c4cc(F)cc(c34)N[C@@H]2c2c(F)cc(F)cc2F)CC1 ; CN1CCC([C@@H]2c3n[nH]c(=O)c4cc(F)cc(c34)N[C@H]2c2c(F)cc(F)cc2F)CC1] 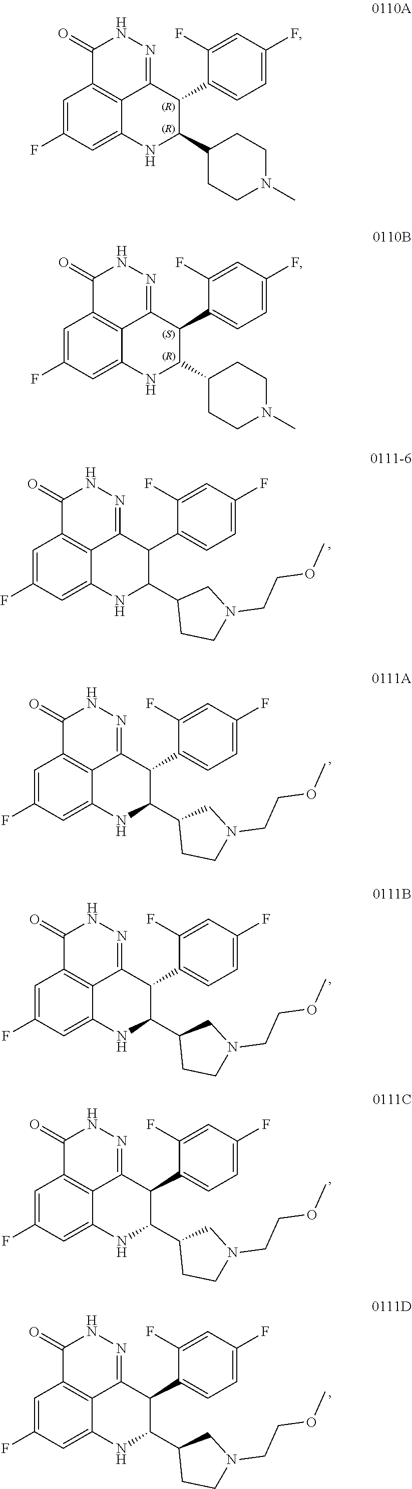 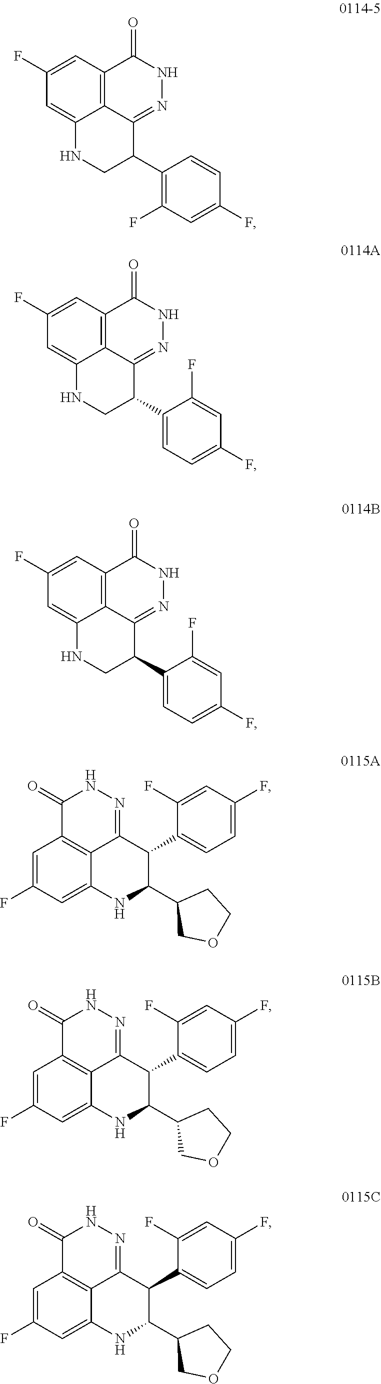 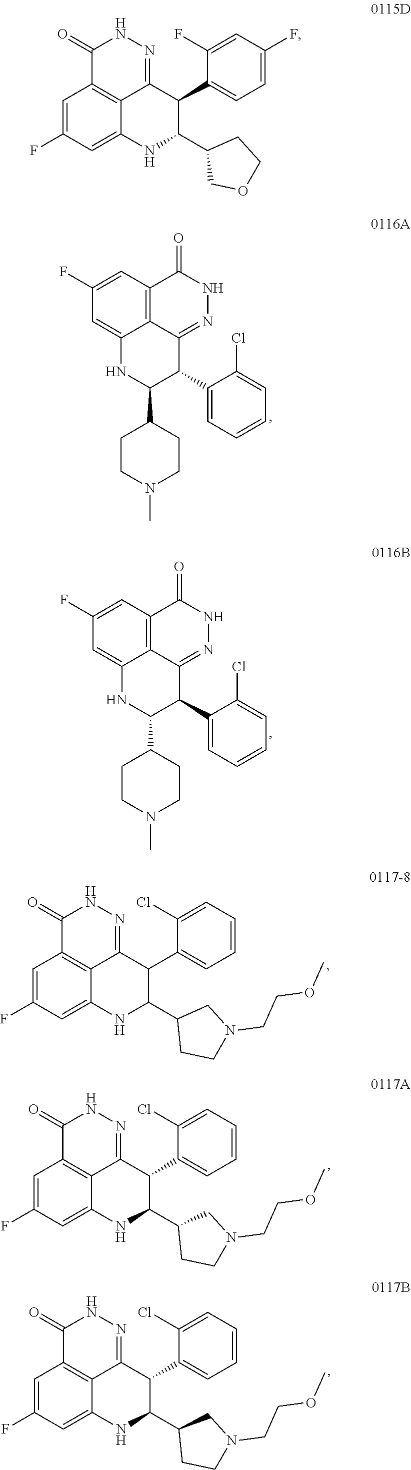 [SMILES: O=c1[nH]nc2c3c(cc(F)cc13)N[C@@H]([C@@H]1CCOC1)[C@@H]2c1ccc(F)cc1F ; CN1CCC([C@H]2Nc3cc(F)cc4c(=O)[nH]nc(c34)[C@@H]2c2ccccc2Cl)CC1 ; CN1CCC([C@@H]2Nc3cc(F)cc4c(=O)[nH]nc(c34)[C@H]2c2ccccc2Cl)CC1 ; COCCN1CCC(C2Nc3cc(F)cc4c(=O)[nH]nc(c34)C2c2ccccc2Cl)C1 ; COCCN1CC[C@H]([C@H]2Nc3cc(F)cc4c(=O)[nH]nc(c34)[C@@H]2c2ccccc2Cl)C1 ; COCCN1CC[C@@H]([C@H]2Nc3cc(F)cc4c(=O)[nH]nc(c34)[C@@H]2c2ccccc2Cl)C1] 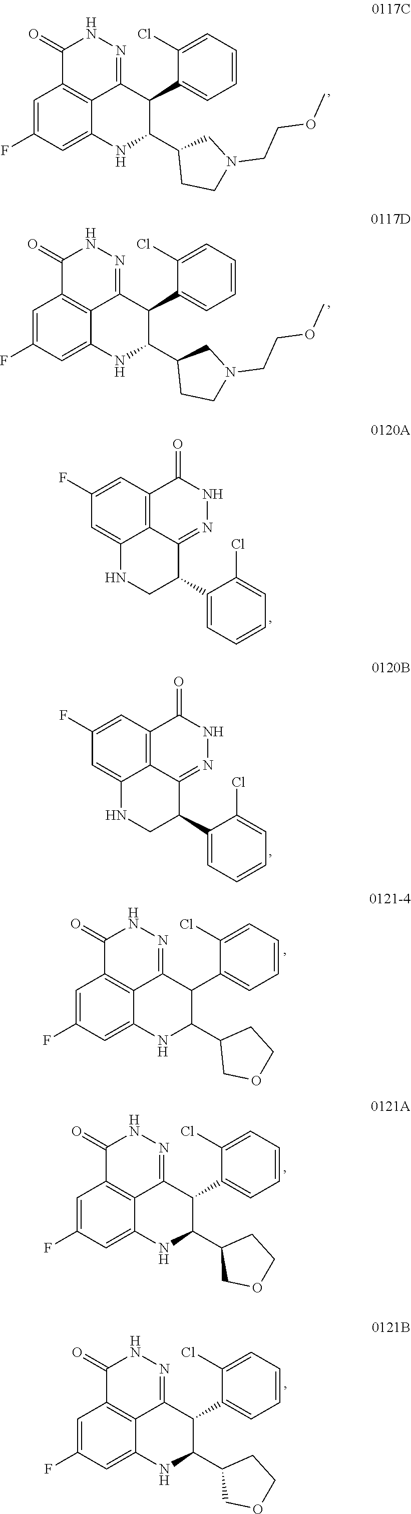 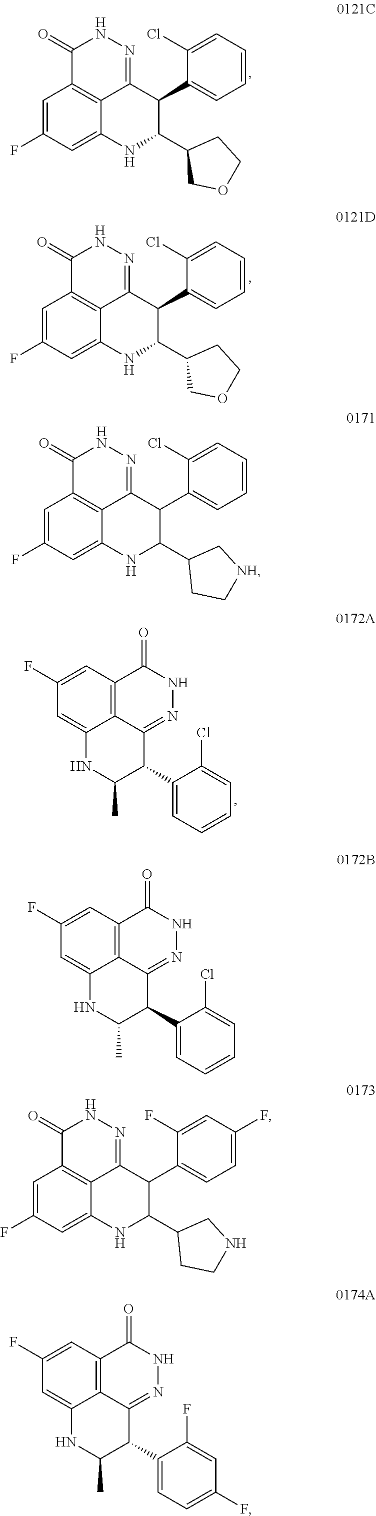 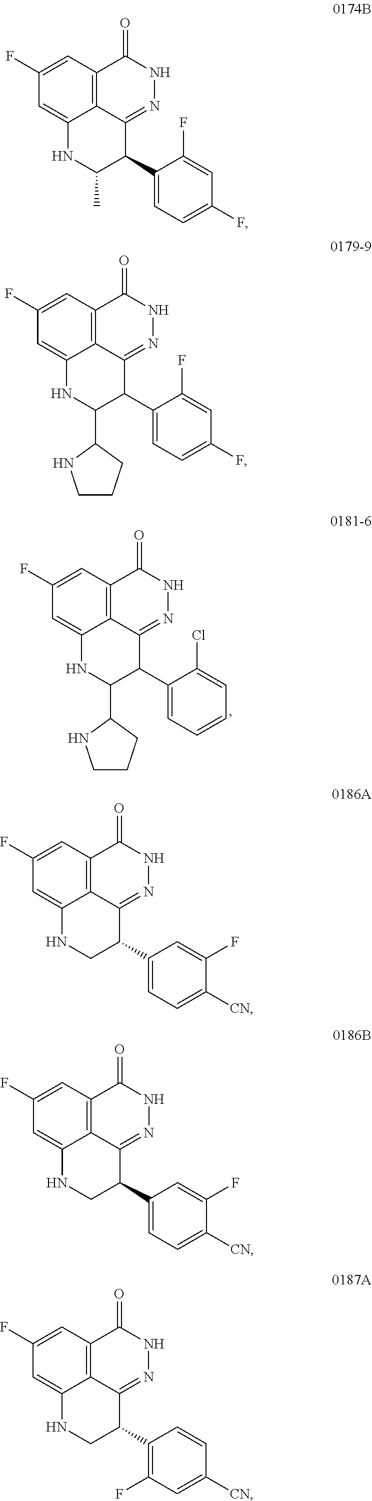 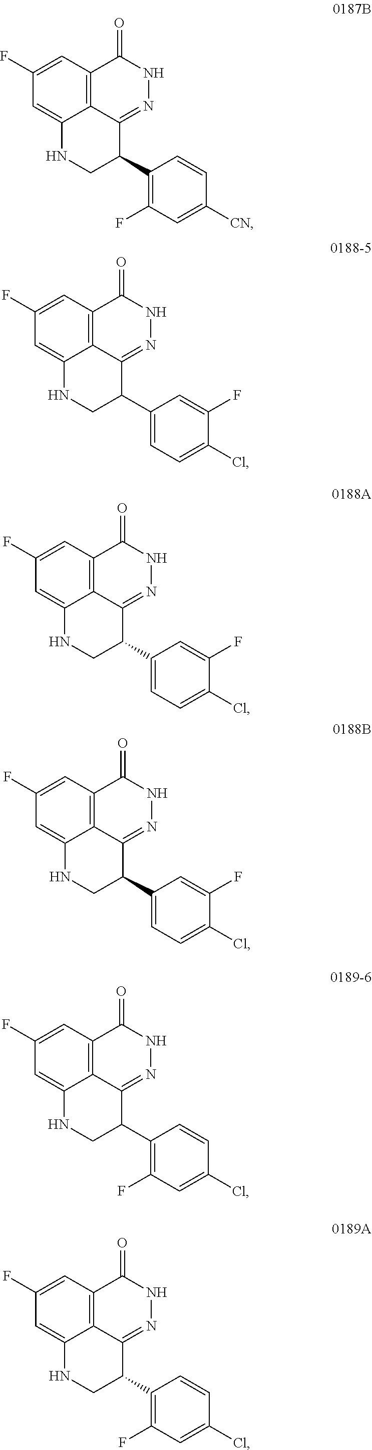 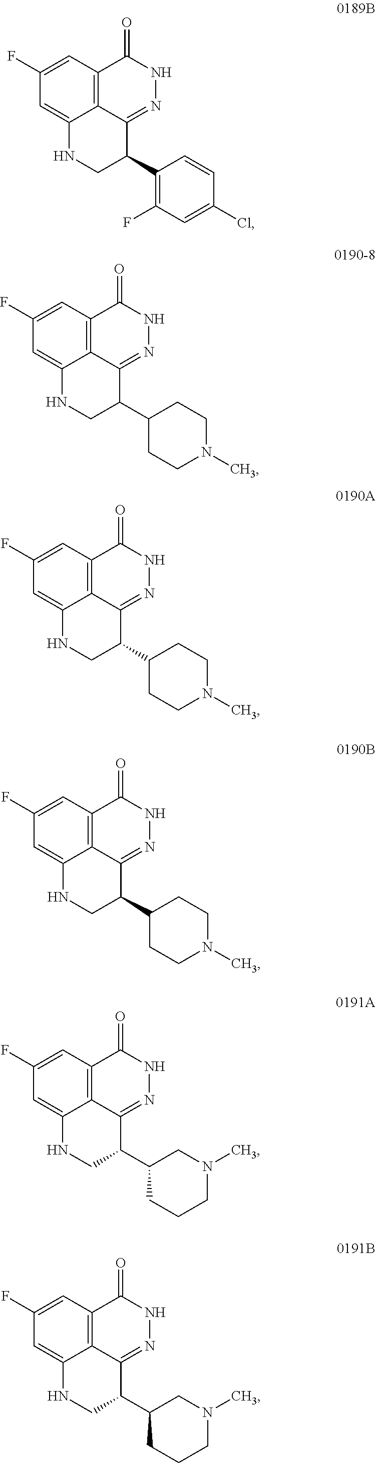 [SMILES: O=c1[nH]nc2c3c(cc(F)cc13)NC[C@@H]2c1ccc(Cl)cc1F ; CN1CCC(C2CNc3cc(F)cc4c(=O)[nH]nc2c34)CC1 ; CN1CCC([C@@H]2CNc3cc(F)cc4c(=O)[nH]nc2c34)CC1 ; CN1CCC([C@H]2CNc3cc(F)cc4c(=O)[nH]nc2c34)CC1 ; CN1CCC[C@H]([C@@H]2CNc3cc(F)cc4c(=O)[nH]nc2c34)C1 ; CN1CCC[C@@H]([C@@H]2CNc3cc(F)cc4c(=O)[nH]nc2c34)C1] 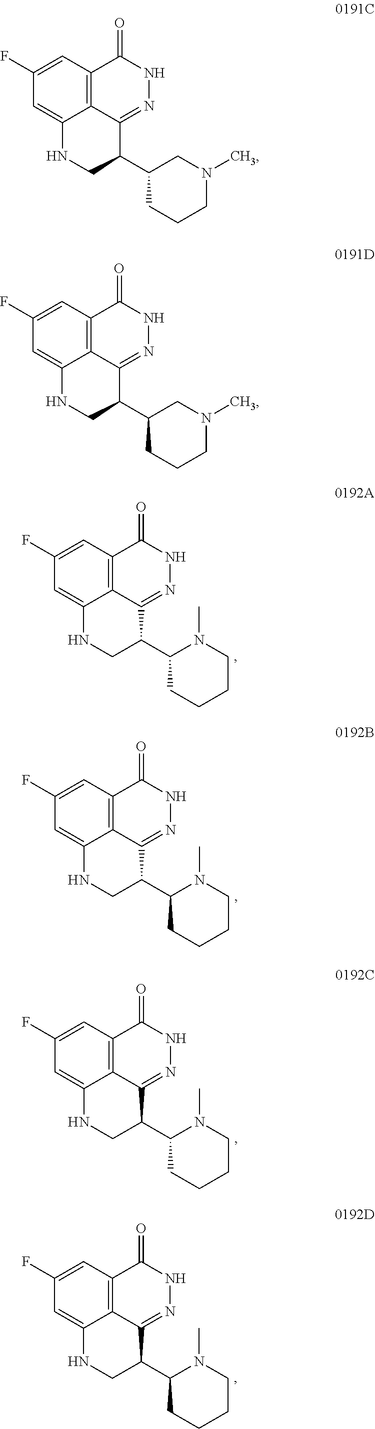 [SMILES: CN1CCC[C@H]([C@H]2CNc3cc(F)cc4c(=O)[nH]nc2c34)C1 ; CN1CCC[C@@H]([C@H]2CNc3cc(F)cc4c(=O)[nH]nc2c34)C1 ; CN1CCCC[C@@H]1[C@@H]1CNc2cc(F)cc3c(=O)[nH]nc1c23 ; CN1CCCC[C@H]1[C@@H]1CNc2cc(F)cc3c(=O)[nH]nc1c23 ; CN1CCCC[C@@H]1[C@H]1CNc2cc(F)cc3c(=O)[nH]nc1c23 ; CN1CCCC[C@H]1[C@H]1CNc2cc(F)cc3c(=O)[nH]nc1c23] 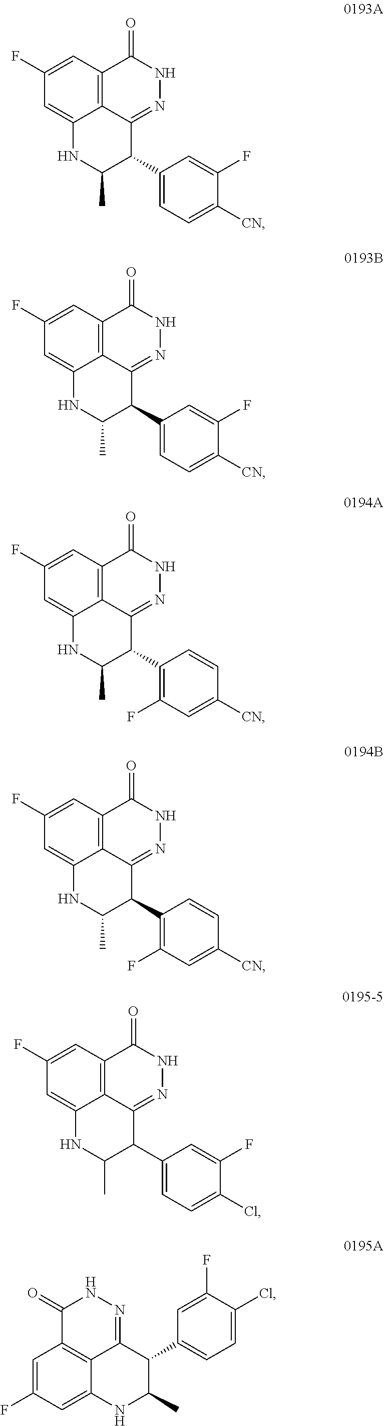 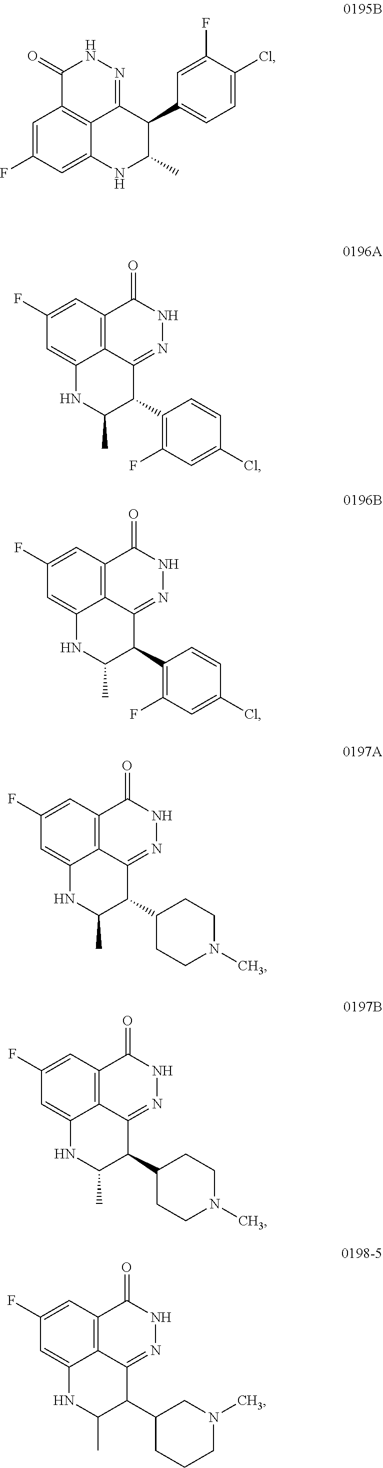 [SMILES: C[C@@H]1Nc2cc(F)cc3c(=O)[nH]nc(c23)[C@H]1c1ccc(Cl)c(F)c1 ; C[C@H]1Nc2cc(F)cc3c(=O)[nH]nc(c23)[C@@H]1c1ccc(Cl)cc1F ; C[C@@H]1Nc2cc(F)cc3c(=O)[nH]nc(c23)[C@H]1c1ccc(Cl)cc1F ; C[C@H]1Nc2cc(F)cc3c(=O)[nH]nc(c23)[C@@H]1C1CCN(C)CC1 ; C[C@@H]1Nc2cc(F)cc3c(=O)[nH]nc(c23)[C@H]1C1CCN(C)CC1 ; CC1Nc2cc(F)cc3c(=O)[nH]nc(c23)C1C1CCCN(C)C1] 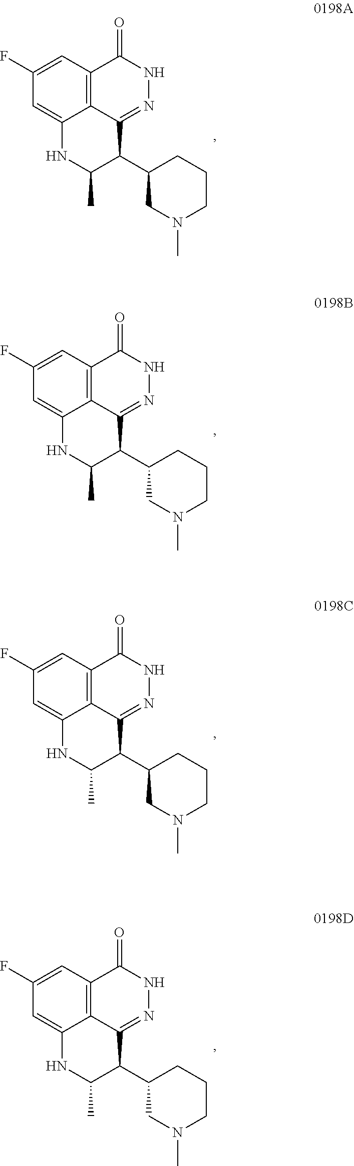 [SMILES: C[C@H]1Nc2cc(F)cc3c(=O)[nH]nc(c23)[C@H]1[C@H]1CCCN(C)C1 ; C[C@H]1Nc2cc(F)cc3c(=O)[nH]nc(c23)[C@H]1[C@@H]1CCCN(C)C1 ; C[C@@H]1Nc2cc(F)cc3c(=O)[nH]nc(c23)[C@H]1[C@H]1CCCN(C)C1 ; C[C@@H]1Nc2cc(F)cc3c(=O)[nH]nc(c23)[C@H]1[C@@H]1CCCN(C)C1] 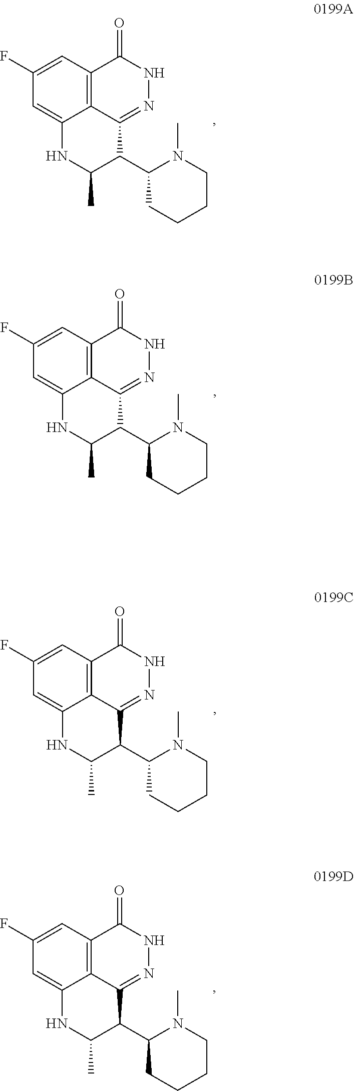 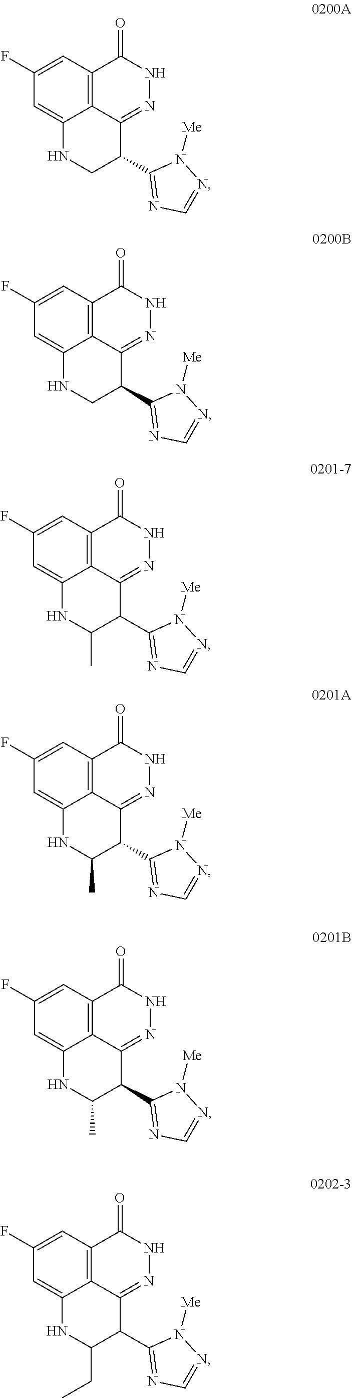 [SMILES: Cn1ncnc1[C@@H]1CNc2cc(F)cc3c(=O)[nH]nc1c23 ; Cn1ncnc1[C@H]1CNc2cc(F)cc3c(=O)[nH]nc1c23 ; CC1Nc2cc(F)cc3c(=O)[nH]nc(c23)C1c1ncnn1C ; C[C@H]1Nc2cc(F)cc3c(=O)[nH]nc(c23)[C@@H]1c1ncnn1C ; C[C@@H]1Nc2cc(F)cc3c(=O)[nH]nc(c23)[C@H]1c1ncnn1C ; CCC1Nc2cc(F)cc3c(=O)[nH]nc(c23)C1c1ncnn1C] 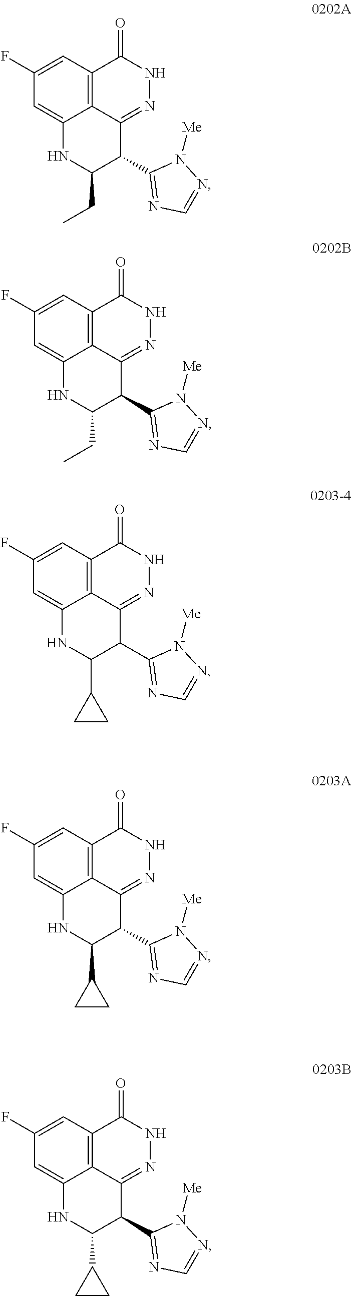 [SMILES: CC[C@H]1Nc2cc(F)cc3c(=O)[nH]nc(c23)[C@@H]1c1ncnn1C ; CC[C@@H]1Nc2cc(F)cc3c(=O)[nH]nc(c23)[C@H]1c1ncnn1C ; Cn1ncnc1C1c2n[nH]c(=O)c3cc(F)cc(c23)NC1C1CC1 ; Cn1ncnc1[C@H]1c2n[nH]c(=O)c3cc(F)cc(c23)N[C@@H]1C1CC1 ; Cn1ncnc1[C@@H]1c2n[nH]c(=O)c3cc(F)cc(c23)N[C@H]1C1CC1] 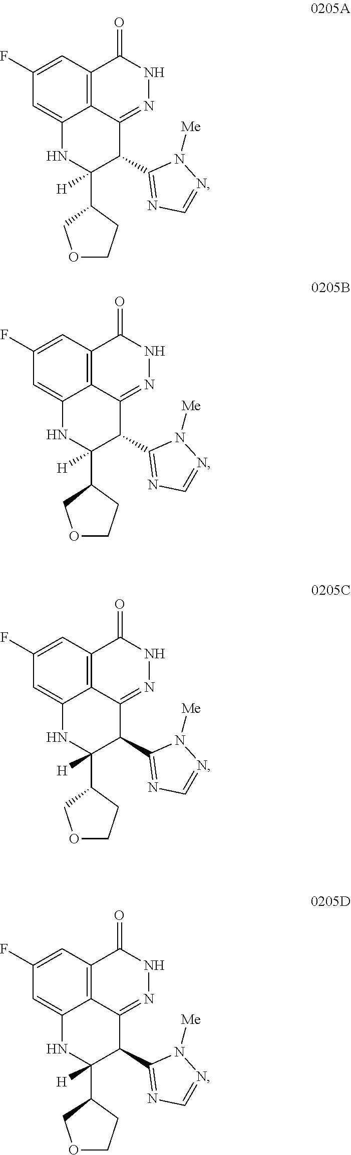 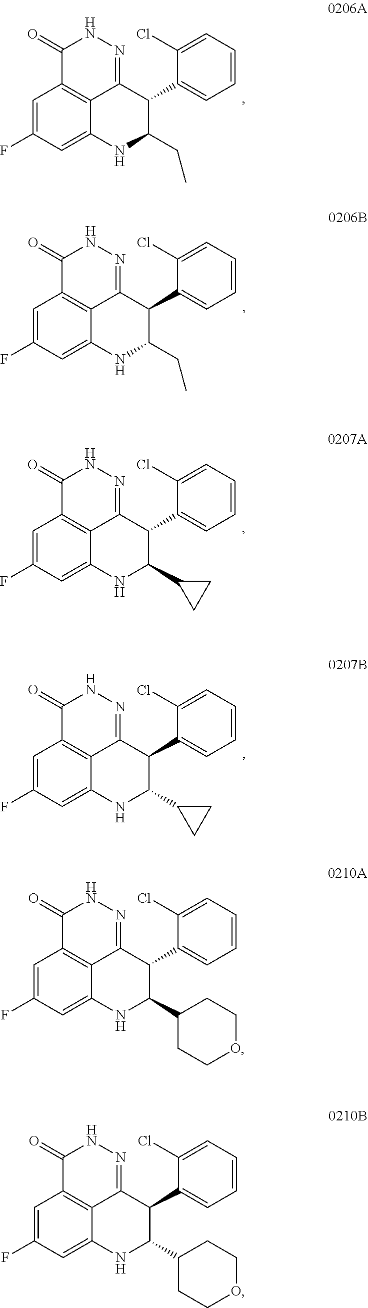 [SMILES: CC[C@H]1Nc2cc(F)cc3c(=O)[nH]nc(c23)[C@@H]1c1ccccc1Cl ; CC[C@@H]1Nc2cc(F)cc3c(=O)[nH]nc(c23)[C@H]1c1ccccc1Cl ; O=c1[nH]nc2c3c(cc(F)cc13)N[C@H](C1CC1)[C@H]2c1ccccc1Cl ; O=c1[nH]nc2c3c(cc(F)cc13)N[C@@H](C1CC1)[C@@H]2c1ccccc1Cl ; O=c1[nH]nc2c3c(cc(F)cc13)N[C@H](C1CCOCC1)[C@H]2c1ccccc1Cl ; O=c1[nH]nc2c3c(cc(F)cc13)N[C@@H](C1CCOCC1)[C@@H]2c1ccccc1Cl] 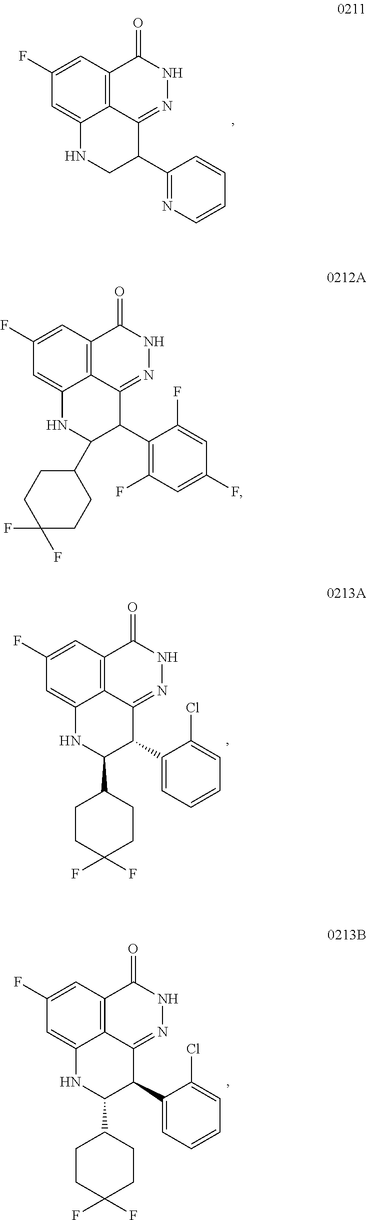 [SMILES: O=c1[nH]nc2c3c(cc(F)cc13)NCC2c1ccccn1 ; O=c1[nH]nc2c3c(cc(F)cc13)NC(C1CCC(F)(F)CC1)C2c1c(F)cc(F)cc1F ; O=c1[nH]nc2c3c(cc(F)cc13)N[C@H](C1CCC(F)(F)CC1)[C@H]2c1ccccc1Cl ; O=c1[nH]nc2c3c(cc(F)cc13)N[C@@H](C1CCC(F)(F)CC1)[C@@H]2c1ccccc1Cl] 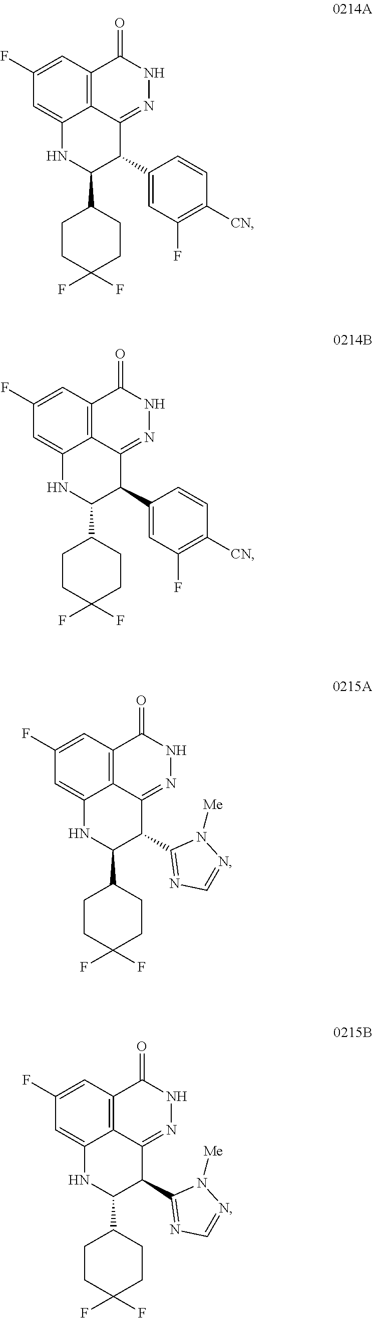 [SMILES: N#Cc1ccc([C@H]2c3n[nH]c(=O)c4cc(F)cc(c34)N[C@@H]2C2CCC(F)(F)CC2)cc1F ; N#Cc1ccc([C@@H]2c3n[nH]c(=O)c4cc(F)cc(c34)N[C@H]2C2CCC(F)(F)CC2)cc1F ; Cn1ncnc1[C@H]1c2n[nH]c(=O)c3cc(F)cc(c23)N[C@@H]1C1CCC(F)(F)CC1 ; Cn1ncnc1[C@@H]1c2n[nH]c(=O)c3cc(F)cc(c23)N[C@H]1C1CCC(F)(F)CC1] 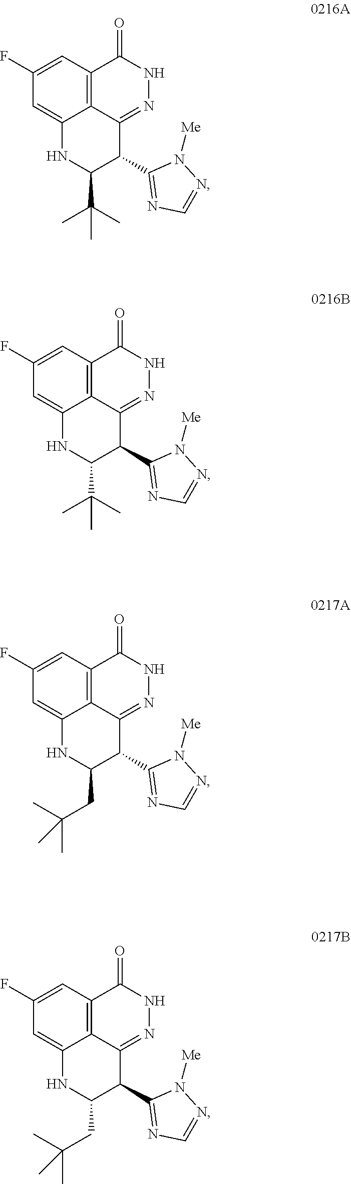 [SMILES: Cn1ncnc1[C@H]1c2n[nH]c(=O)c3cc(F)cc(c23)N[C@@H]1C(C)(C)C ; Cn1ncnc1[C@@H]1c2n[nH]c(=O)c3cc(F)cc(c23)N[C@H]1C(C)(C)C ; Cn1ncnc1[C@H]1c2n[nH]c(=O)c3cc(F)cc(c23)N[C@@H]1CC(C)(C)C ; Cn1ncnc1[C@@H]1c2n[nH]c(=O)c3cc(F)cc(c23)N[C@H]1CC(C)(C)C] 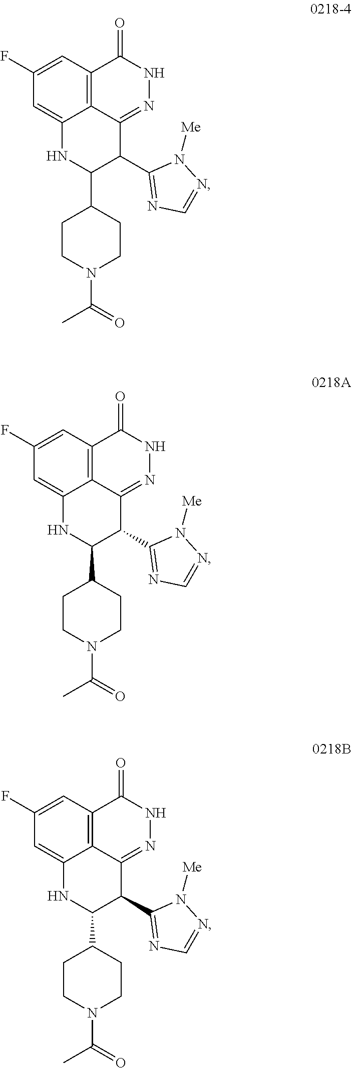 [SMILES: CC(=O)N1CCC(C2Nc3cc(F)cc4c(=O)[nH]nc(c34)C2c2ncnn2C)CC1 ; CC(=O)N1CCC([C@H]2Nc3cc(F)cc4c(=O)[nH]nc(c34)[C@@H]2c2ncnn2C)CC1 ; CC(=O)N1CCC([C@@H]2Nc3cc(F)cc4c(=O)[nH]nc(c34)[C@H]2c2ncnn2C)CC1] 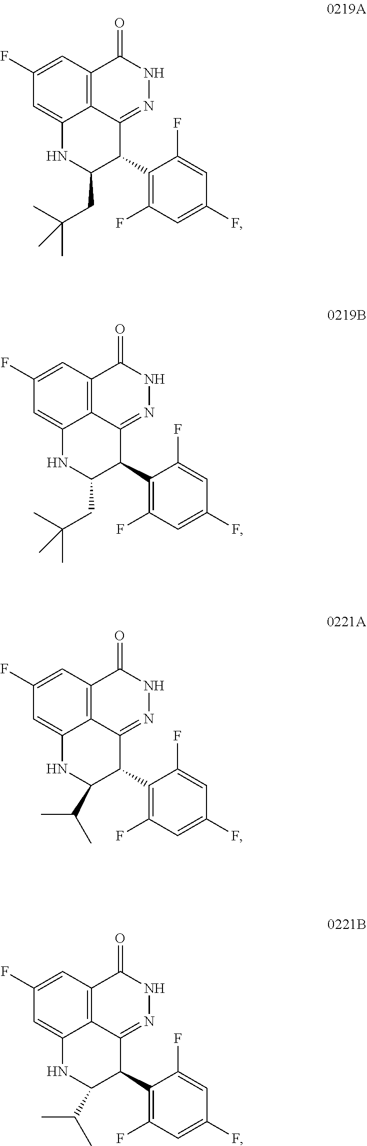 [SMILES: CC(C)(C)C[C@H]1Nc2cc(F)cc3c(=O)[nH]nc(c23)[C@@H]1c1c(F)cc(F)cc1F ; CC(C)(C)C[C@@H]1Nc2cc(F)cc3c(=O)[nH]nc(c23)[C@H]1c1c(F)cc(F)cc1F ; CC(C)[C@H]1Nc2cc(F)cc3c(=O)[nH]nc(c23)[C@@H]1c1c(F)cc(F)cc1F ; CC(C)[C@@H]1Nc2cc(F)cc3c(=O)[nH]nc(c23)[C@H]1c1c(F)cc(F)cc1F] 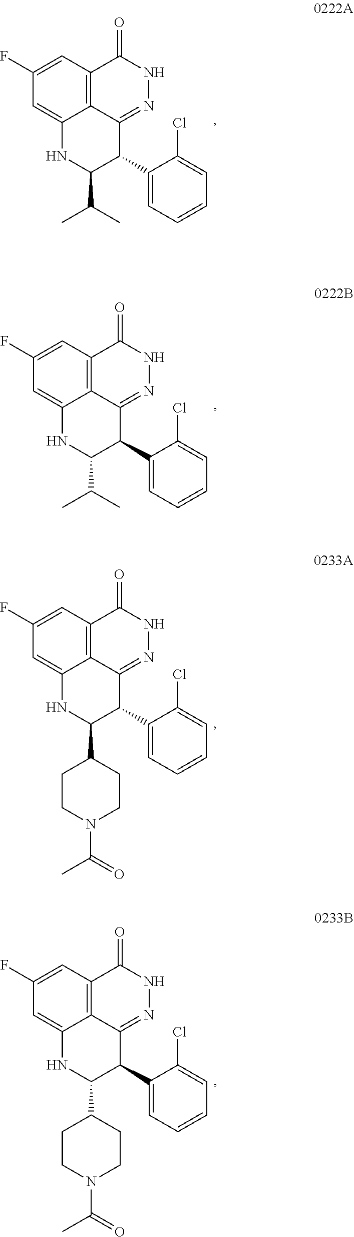 [SMILES: CC(C)[C@H]1Nc2cc(F)cc3c(=O)[nH]nc(c23)[C@@H]1c1ccccc1Cl ; CC(C)[C@@H]1Nc2cc(F)cc3c(=O)[nH]nc(c23)[C@H]1c1ccccc1Cl ; CC(=O)N1CCC([C@H]2Nc3cc(F)cc4c(=O)[nH]nc(c34)[C@@H]2c2ccccc2Cl)CC1 ; CC(=O)N1CCC([C@@H]2Nc3cc(F)cc4c(=O)[nH]nc(c34)[C@H]2c2ccccc2Cl)CC1] 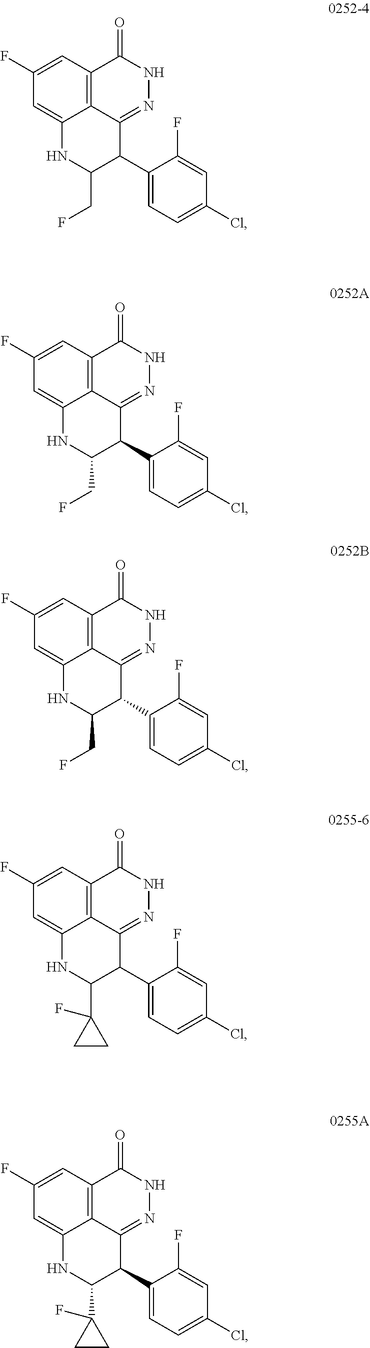 [SMILES: O=c1[nH]nc2c3c(cc(F)cc13)NC(CF)C2c1ccc(Cl)cc1F ; O=c1[nH]nc2c3c(cc(F)cc13)N[C@@H](CF)[C@@H]2c1ccc(Cl)cc1F ; O=c1[nH]nc2c3c(cc(F)cc13)N[C@H](CF)[C@H]2c1ccc(Cl)cc1F ; O=c1[nH]nc2c3c(cc(F)cc13)NC(C1(F)CC1)C2c1ccc(Cl)cc1F ; O=c1[nH]nc2c3c(cc(F)cc13)N[C@@H](C1(F)CC1)[C@@H]2c1ccc(Cl)cc1F] 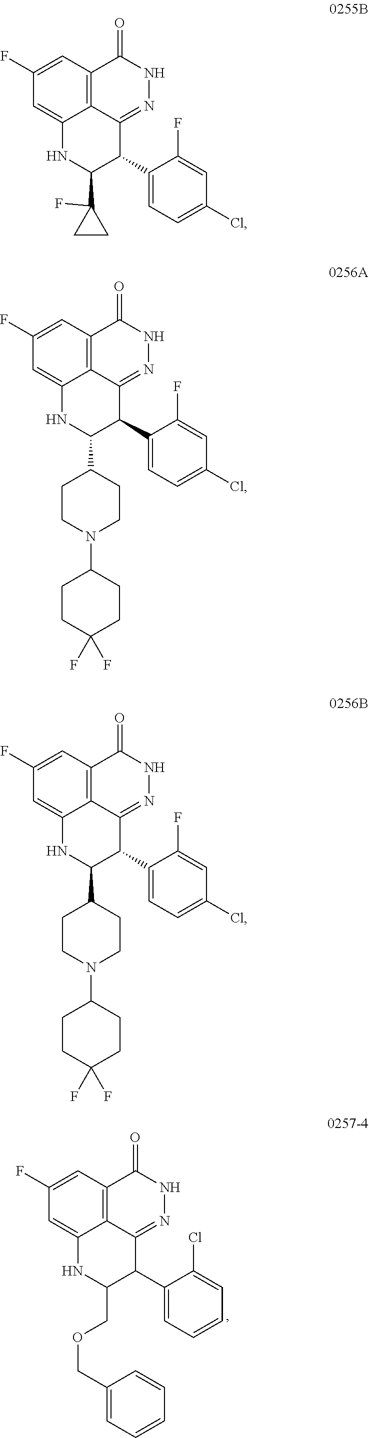 [SMILES: O=c1[nH]nc2c3c(cc(F)cc13)N[C@H](C1(F)CC1)[C@H]2c1ccc(Cl)cc1F ; O=c1[nH]nc2c3c(cc(F)cc13)N[C@@H](C1CCN(C3CCC(F)(F)CC3)CC1)[C@@H]2c1ccc(Cl)cc1F ; O=c1[nH]nc2c3c(cc(F)cc13)N[C@H](C1CCN(C3CCC(F)(F)CC3)CC1)[C@H]2c1ccc(Cl)cc1F ; O=c1[nH]nc2c3c(cc(F)cc13)NC(COCc1ccccc1)C2c1ccccc1Cl] 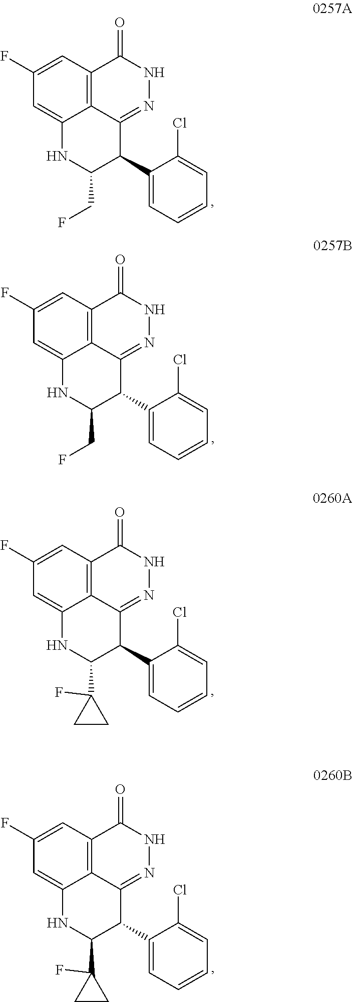 [SMILES: O=c1[nH]nc2c3c(cc(F)cc13)N[C@@H](CF)[C@@H]2c1ccccc1Cl ; O=c1[nH]nc2c3c(cc(F)cc13)N[C@H](CF)[C@H]2c1ccccc1Cl ; O=c1[nH]nc2c3c(cc(F)cc13)N[C@@H](C1(F)CC1)[C@@H]2c1ccccc1Cl ; O=c1[nH]nc2c3c(cc(F)cc13)N[C@H](C1(F)CC1)[C@H]2c1ccccc1Cl] 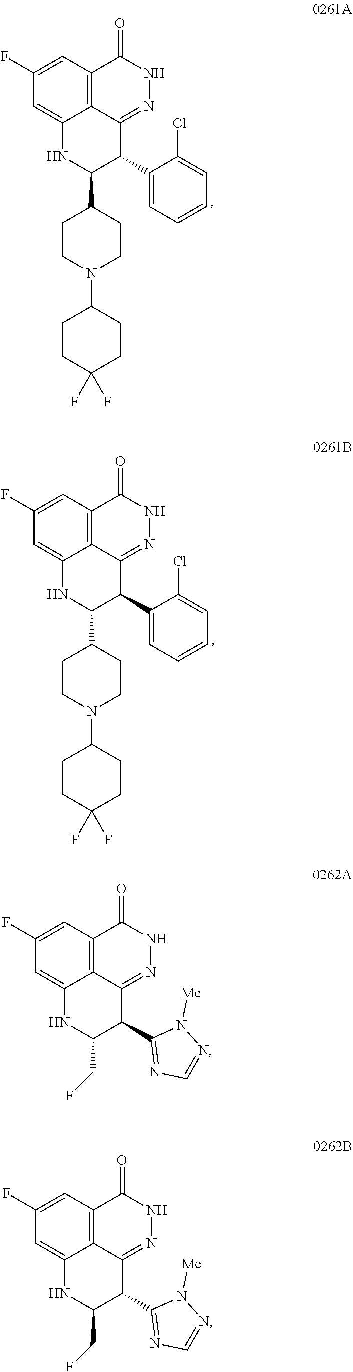 [SMILES: O=c1[nH]nc2c3c(cc(F)cc13)N[C@H](C1CCN(C3CCC(F)(F)CC3)CC1)[C@H]2c1ccccc1Cl ; O=c1[nH]nc2c3c(cc(F)cc13)N[C@@H](C1CCN(C3CCC(F)(F)CC3)CC1)[C@@H]2c1ccccc1Cl ; Cn1ncnc1[C@@H]1c2n[nH]c(=O)c3cc(F)cc(c23)N[C@H]1CF ; Cn1ncnc1[C@H]1c2n[nH]c(=O)c3cc(F)cc(c23)N[C@@H]1CF] 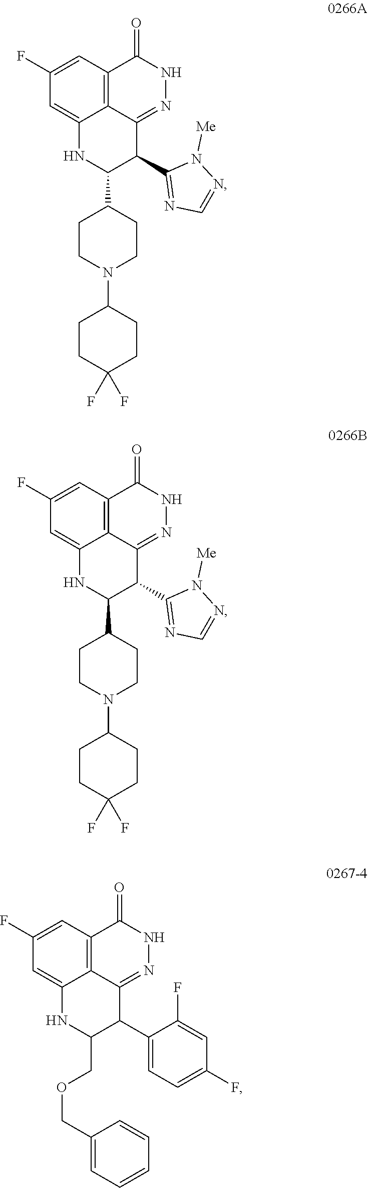 [SMILES: Cn1ncnc1[C@@H]1c2n[nH]c(=O)c3cc(F)cc(c23)N[C@H]1C1CCN(C2CCC(F)(F)CC2)CC1 ; Cn1ncnc1[C@H]1c2n[nH]c(=O)c3cc(F)cc(c23)N[C@@H]1C1CCN(C2CCC(F)(F)CC2)CC1 ; O=c1[nH]nc2c3c(cc(F)cc13)NC(COCc1ccccc1)C2c1ccc(F)cc1F] 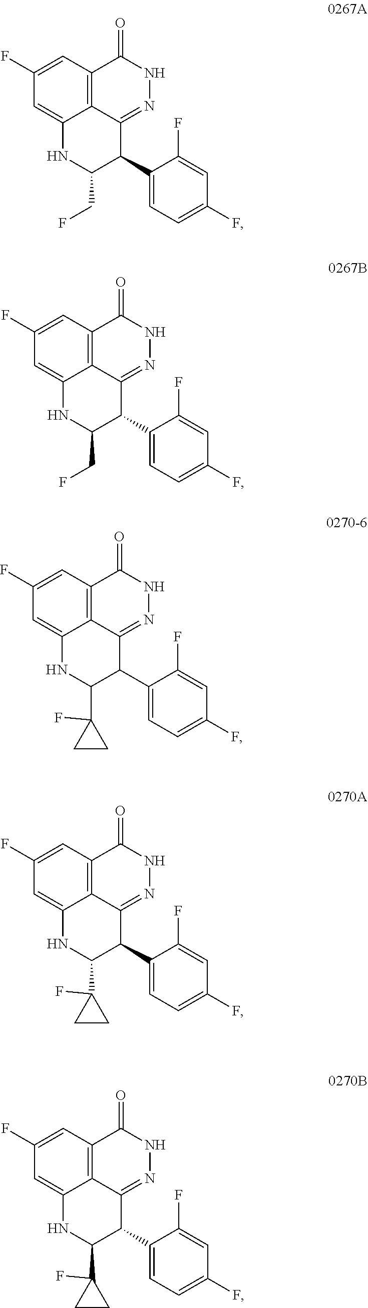 [SMILES: O=c1[nH]nc2c3c(cc(F)cc13)N[C@@H](CF)[C@@H]2c1ccc(F)cc1F ; O=c1[nH]nc2c3c(cc(F)cc13)N[C@H](CF)[C@H]2c1ccc(F)cc1F ; O=c1[nH]nc2c3c(cc(F)cc13)NC(C1(F)CC1)C2c1ccc(F)cc1F ; O=c1[nH]nc2c3c(cc(F)cc13)N[C@@H](C1(F)CC1)[C@@H]2c1ccc(F)cc1F ; O=c1[nH]nc2c3c(cc(F)cc13)N[C@H](C1(F)CC1)[C@H]2c1ccc(F)cc1F] 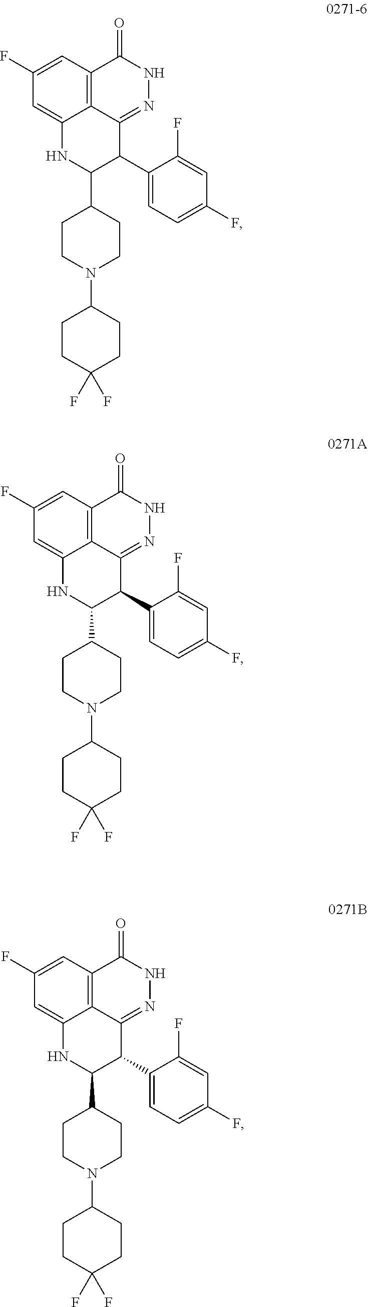 [SMILES: O=c1[nH]nc2c3c(cc(F)cc13)NC(C1CCN(C3CCC(F)(F)CC3)CC1)C2c1ccc(F)cc1F ; O=c1[nH]nc2c3c(cc(F)cc13)N[C@@H](C1CCN(C3CCC(F)(F)CC3)CC1)[C@@H]2c1ccc(F)cc1F ; O=c1[nH]nc2c3c(cc(F)cc13)N[C@H](C1CCN(C3CCC(F)(F)CC3)CC1)[C@H]2c1ccc(F)cc1F] 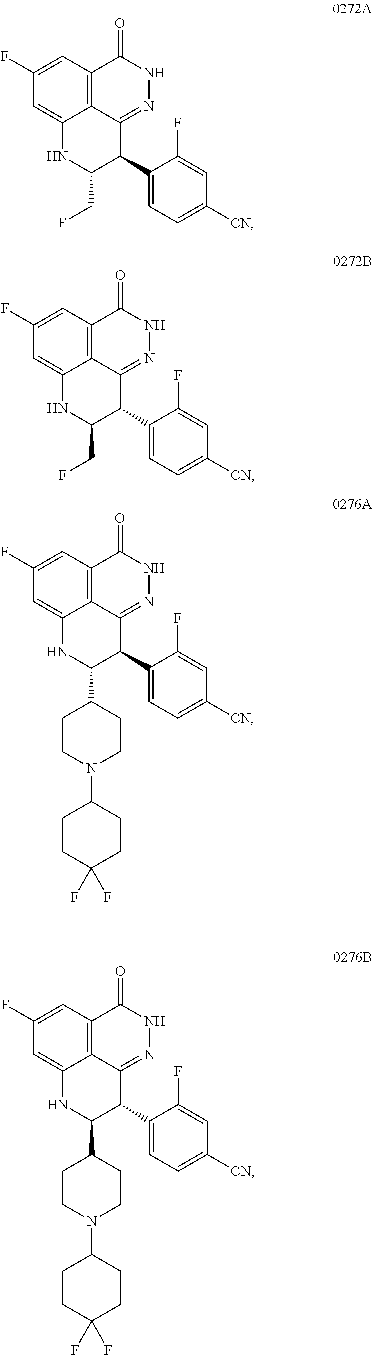 [SMILES: N#Cc1ccc([C@@H]2c3n[nH]c(=O)c4cc(F)cc(c34)N[C@H]2CF)c(F)c1 ; N#Cc1ccc([C@H]2c3n[nH]c(=O)c4cc(F)cc(c34)N[C@@H]2CF)c(F)c1 ; N#Cc1ccc([C@@H]2c3n[nH]c(=O)c4cc(F)cc(c34)N[C@H]2C2CCN(C3CCC(F)(F)CC3)CC2)c(F)c1 ; N#Cc1ccc([C@H]2c3n[nH]c(=O)c4cc(F)cc(c34)N[C@@H]2C2CCN(C3CCC(F)(F)CC3)CC2)c(F)c1]
or a pharmaceutically acceptable salt and/or solvate of any one thereof.
